(19) United States
(12) Patent Application Publication
(10) Pub. No.: US 2026/0185061 A1
(43) Pub. Date: Jul. 2, 2026

(54) SERINE RECOMBINASES FOR GENE EDITING

(71) Applicant: Metagenomi, Inc., Emeryville, CA (US)

(72) Inventors: Brian C. THOMAS, Emeryville, CA (US); Christopher BROWN, Emeryville, CA (US); Cindy CASTELLE, Emeryville, CA (US); Gregory J. COST, Emeryville, CA (US); Daniela S.A. GOLTSMAN, Emeryville, CA (US); Khak Khak KHAYI, Emeryville, CA (US); Sarah LAPERRIERE, Emeryville, CA (US); Jason LIU, Emeryville, CA (US); Soungsothira TOCH, Emeryville, CA (US)

(21) Appl. No.: 19/126,597

(22) PCT Filed: Nov. 6, 2023
(86) PCT No.: PCT/US2023/078853

§ 371 Date: May 1, 2025

Related U.S. Application Data
(60) Provisional application No. 63/382,690, filed on Nov. 7, 2022.
(60) Provisional application No. 63/510,567, filed on Jun. 27, 2023.

(51) Int. Cl. C12N 9/22; C12N 15/85; C12N 15/90

(52) U.S. Cl. CPC C12N 9/22; C12N 15/85; C12N 15/907

(57) ABSTRACT

The disclosure relates to gene editing systems comprising serine recombinases and methods of using such serine recombinases for integration of nucleic acid sequences. More specifically, the disclosure relates to sequence-defined serine recombinases having attachment sites such as a bacterial genomic recombination sequence (attB). Methods are also provided for recombinant pro-duction of said recombinases.

### CROSS-REFERENCE

This application claims the benefit of and priority to U.S. Provisional Patent Application No. 63/382,690, filed Nov. 7, 2022, and U.S. Provisional Patent Application No. 63/510,567 filed Jun. 27, 2023, each of which is incorporated by reference in its entirety herein.

### BRIEF SUMMARY

The disclosure is based, in part, upon the development of serine recombinases for use in gene editing systems to integrate nucleic acid sequences.

Described herein are gene editing systems comprising: a) a serine recombinase comprising at least about 80% sequence identity to any one of SEQ ID NOs: 21-7060, 7105-7142 and 7211-7214 or a nucleic acid encoding the serine recombinase; and b) a nucleic acid comprising a donor polynucleotide and a first attachment site sequence. In some embodiments, the first attachment site sequence is 5′ of the donor polynucleotide. In some embodiments, the nucleic acid encoding the serine recombinase further comprises a second attachment site sequence. In some embodiments, the second attachment site sequence is 5′ of the serine recombinase. In some embodiments, the first attachment site sequence and the second attachment site sequence are capable of recombination. In some embodiments, the first attachment site sequence is a bacterial genomic recombination sequence (attB). In some embodiments, the first attachment site sequence is a phage genomic recombination sequence (attP). In some embodiments, the second attachment site sequence is a bacterial genomic recombination sequence (attB). In some embodiments, the second attachment site sequence is a phage genomic recombination sequence (attP). In some embodiments, the attB sequence comprises about 20 to about 500 nucleotides. In some embodiments, the attP sequence comprises about 20 to about 500 nucleotides. In some embodiments, the attB sequence comprises at least about 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least about 80% sequence identity to any one of SEQ ID NOs: 1, 5, 9, and 13. In some embodiments, the attP sequence comprises at least about 80% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least about 80% sequence identity to any one of SEQ ID NOs: 2, 6, 10, and 14. In some embodiments, the nucleic acid comprising the donor polynucleotide and the first attachment sequence is delivered by a plasmid, a nanoplasmid, a phagemid, a phage derivative, a virus, a bacmid, a bacterial artificial chromosome (BAC), a minicircle, a doggybone, a yeast artificial chromosome (YAC), or a cosmid. In some embodiments, the nucleic acid encoding the serine recombinase is delivered by a plasmid, a nanoplasmid, a phagemid, a phage derivative, a virus, a bacmid, a bacterial artificial chromosome (BAC), a minicircle, a doggybone, a yeast artificial chromosome (YAC), or a cosmid. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-21YF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the herpesvirus is HSV-1, HSV-2, VZV, EBV, CMV, HHV-6, HHV-7, or HHV-8. In some embodiments, the donor polynucleotide comprises a size of at least about 1 kilobase (kb), 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 110 kb, 120 kb, or more than 120 kb. In some embodiments, the donor polynucleotide encodes a therapeutic, a reporter, or a marker. In some embodiments, the reporter comprises a fluorescent protein. In some embodiments, the fluorescent protein is GFP, EBFP, EBFP2, Azurite, mKalamal, ECFP, Cerulean, CyPet, YFP, Citrine, Venus, YPet, RFP, CFP, or a derivative thereof. In some embodiments, the reporter is acetohydroxyacid synthase (AHAS), alkaline phosphatase (AP), beta galactosidase (LacZ), beta glucuronidase (GUS), chloramphenicol acetyltransferase (CAT), horseradish peroxidase (HRP), luciferase (Luc), nopaline synthase (NOS), octopine synthase (OCS), luciferase, or a derivative thereof. In some embodiments, the marker is an antibiotic resistance marker. In some embodiments, the antibiotic resistance marker is kanamycin, spectinomycin, streptomycin, ampicillin, carbenicillin, bleomycin, erythromycin, polymyxin B, tetracycline, chloramphenicol, neomycin, zeocin, or a derivative thereof. In some embodiments, the marker is a cell surface marker.

Described herein are eukaryotic genomes comprising a donor polynucleotide sequence; and an attL sequence 5′ to the donor polynucleotide sequence, wherein the attL sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC. In some embodiments, the eukaryotic genome further comprises an attR sequence 3′ to the donor polynucleotide sequence.

Described herein are eukaryotic genomes comprising a donor polynucleotide sequence; and an attL sequence 3′ to the donor polynucleotide sequence, wherein the attL sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC. In some embodiments, the eukaryotic genome further comprises an attR sequence 3′ to the donor polynucleotide sequence.

Described herein are eukaryotic genomes comprising: a donor polynucleotide sequence; an attL sequence 5′ or 3′ to the donor polynucleotide sequence, wherein the attL sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC; and an attR sequence 5′ or 3′ to the donor polynucleotide sequence, wherein the attR sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC. In some embodiments, the attL sequence and the attR sequence are the same. In some embodiments, the attL sequence is a recombined sequence of a first attachment site sequence and a second attachment site sequence. In some embodiments, the attR sequence is a recombined sequence of a first attachment site sequence and a second attachment site sequence. In some embodiments, the first attachment site sequence is a bacterial genomic recombination sequence (attB). In some embodiments, the first attachment site sequence is a phage genomic recombination sequence (attP). In some embodiments, the second attachment site sequence is a bacterial genomic recombination sequence (attB). In some embodiments, the second attachment site sequence is a phage genomic recombination sequence (attP). In some embodiments, the attB sequence comprises about 20 to about 500 nucleotides. In some embodiments, the attP sequence comprises about 20 to about 500 nucleotides. In some embodiments, the attB sequence comprises at least about 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least about 80% sequence identity to any one of SEQ ID Nos: 1, 5, 9, and 13. In some embodiments, the attP sequence comprises at least about 80% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least about 80% sequence identity to any one of SEQ ID NOs: 2, 6, 10, and 14. In some embodiments, the attL sequence comprises at least about 80% sequence identity to any one of SEQ ID NOs: 3, 4, 7, 8, 11, 12, 15, 16, 7153, 7157, 7161, 7165, 7169, 7173, 7177, and 7181. In some embodiments, the attR sequence comprises at least about 80% sequence identity to any one of SEQ ID NOs: 3, 4, 7, 8, 11, 12, 15, 16, 7154, 7158, 7162, 7166, 7170, 7174, 7178, and 7182.

Described herein are mammalian cells comprising the eukaryotic genomes described herein. In some embodiments, the mammalian cell is a human cell. In some embodiments, the mammalian cell further comprises a serine recombinase. In some embodiments, the serine recombinase comprises at least about 80% sequence identity to any one of SEQ ID NOs: 21-7060, 7105-7142, and 7211-7214. In some embodiments, the serine recombinase comprises at least about 80% sequence identity to SEQ ID NO: 21. In some embodiments, the serine recombinase comprises at least about 80% sequence identity to SEQ ID NO: 22. In some embodiments, the serine recombinase comprises at least about 80% sequence identity to SEQ ID NO: 23. In some embodiments, the serine recombinase comprises at least about 80% sequence identity to SEQ ID NO: 24. In some embodiments, the serine recombinase comprises an integration efficiency of at least about 5%. In some embodiments, the serine recombinase comprises an integration efficiency of at least about 25%. In some embodiments, the serine recombinase comprises an integration efficiency of at least about 50%. In some embodiments, the serine recombinase is capable of targeting genes comprising a catalase domain or synthase domain. In some embodiments, the catalase is manganese catalase. In some embodiments, the synthase is Queuosine synthase. In some embodiments, the serine recombinase is capable of targeting genes comprising a DUF4244 Pfam domain.

Described herein are eukaryotic cells comprising a serine recombinase comprising at least about 80% sequence identity to any one of SEQ ID NOs: 21-7060 and 7105-7142.

Described herein are eukaryotic cells comprising a serine recombinase comprising at least about 80% sequence identity to SEQ ID NO: 21.

Described herein are eukaryotic cells comprising a serine recombinase comprising at least about 80% sequence identity to SEQ ID NO: 22.

Described herein are eukaryotic cells comprising a serine recombinase comprising at least about 80% sequence identity to SEQ ID NO: 23.

Described herein are eukaryotic cells comprising a serine recombinase comprising at least about 80% sequence identity to SEQ ID NO: 24.

Described herein are eukaryotic cells comprising a serine recombinase comprising at least about 80% sequence identity to SEQ ID NO: 1848.

Described herein are eukaryotic cells comprising a serine recombinase comprising at least about 80% sequence identity to SEQ ID NO: 7111.

Described herein are eukaryotic cells comprising a serine recombinase comprising at least about 80% sequence identity to SEQ ID NO: 7115.

Described herein are eukaryotic cells comprising a serine recombinase comprising at least about 80% sequence identity to SEQ ID NO: 7131.

Described herein are eukaryotic cells comprising a serine recombinase comprising at least about 80% sequence identity to SEQ ID NO: 7136.

Described herein are eukaryotic cells comprising a serine recombinase comprising at least about 80% sequence identity to SEQ ID NO: 7139.

Described herein are eukaryotic cells comprising a serine recombinase comprising at least about 80% sequence identity to SEQ ID NO: 7140.

In some embodiments, the eukaryotic cell is a mammalian cell. In some embodiments, the eukaryotic cell is a human cell.

Described herein are vectors comprising: a) a nucleic acid encoding a serine recombinase comprising at least about 80% sequence identity to any one of SEQ ID NOs: 21-7060 and 7105-7142; and b) one or more regulatory elements. In some embodiments, the one or more regulatory elements comprises a promoter, an enhancer, an intron, a microRNA, a linker, a splicing element, or a polyA signal. In some embodiments, the promoter is selected from a constitutive promoter, an inducible promoter, a mini promoter, or a derivative thereof. In some embodiments, the promoter is selected from the group consisting of: CMV, CBA, EF1a, CAG, PGK, TRE, U6, UAS, T7, Sp6, lac, araBad, trp, Ptac, p5, p19, p40, Synapsin, CaMKII, GRK1, polH, EM7, OpIE1, and a derivative thereof.

Described herein are vectors comprising a nucleic acid encoding a serine recombinase comprising at least about 80% sequence identity to any one of SEQ ID NOs: 21-7060 and 7105-7142, wherein the vector is selected from the group consisting of: a plasmid, a nanoplasmid, a phagemid, a phage derivative, a bacmid, a bacterial artificial chromosome (BAC), a minicircle, a doggybone, a yeast artificial chromosome (YAC), and a cosmid.

Described herein are methods for gene editing, comprising: a) providing or identifying a first attachment site sequence in a host genome; b) providing a nucleic acid comprising a donor polynucleotide and a second attachment site sequence to a host cell; and c) contacting the host cell with a serine recombinase comprising at least about 80% sequence identity to any one of SEQ ID NOs: 21-7060 and 7105-7142 or a nucleic acid encoding the serine recombinase, wherein the first attachment site sequence and the second attachment site sequence are capable of recombination. In some embodiments, the first attachment site sequence is endogenous in the host genome. In some embodiments, the first attachment site sequence is provided using viral delivery. In some embodiments, the first attachment site sequence is provided using a transposase. In some embodiments, the first attachment site sequence is provided using a nuclease. In some embodiments, the nuclease is a double-strand nuclease. In some embodiments, the nuclease is a Type II CRISPR endonuclease. In some embodiments, the nuclease is a Type V CRISPR endonuclease. In some embodiments, the nuclease is Cas9. In some embodiments, the first attachment site sequence is provided using a reverse transcriptase. In some embodiments, the second attachment site sequence is 5′ of the donor polynucleotide. In some embodiments, the first attachment site sequence is a bacterial genomic recombination sequence (attB). In some embodiments, the first attachment site sequence is a phage genomic recombination sequence (attP). In some embodiments, the second attachment site sequence is a bacterial genomic recombination sequence (attB). In some embodiments, the second attachment site sequence is a phage genomic recombination sequence (attP). In some embodiments, the attB sequence comprises about 20 to about 500 nucleotides. In some embodiments, the attP sequence comprises about 20 to about 500 nucleotides. In some embodiments, the attB sequence comprises at least about 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least about 80% sequence identity to any one of SEQ ID NOs: 1, 5, 9, and 13. In some embodiments, the attP sequence comprises at least about 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attP sequence comprises at least about 80% sequence identity to any one of SEQ ID NOs: 2, 6, 10, and 14. In some embodiments, the nucleic acid comprising the donor polynucleotide and the second attachment site sequence is delivered by a plasmid, a nanoplasmid, a phagemid, a phage derivative, a virus, a bacmid, a bacterial artificial chromosome (BAC), a minicircle, a doggybone, a yeast artificial chromosome (YAC), or a cosmid. In some embodiments, the nucleic acid encoding the serine recombinase is delivered by a plasmid, a nanoplasmid, a phagemid, a phage derivative, a virus, a bacmid, a bacterial artificial chromosome (BAC), a minicircle, a doggybone, a yeast artificial chromosome (YAC), or a cosmid. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof. In some embodiments, the herpesvirus is HSV-1, HSV-2, VZV, EBV, CMV, HHV-6, HHV-7, or HHV-8. In some embodiments, the donor polynucleotide comprises a size of at least about 1 kilobase (kb), 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 110 kb, 120 kb, or more than 120 kb. In some embodiments, the donor polynucleotide encodes a therapeutic, a reporter, or a marker. In some embodiments, the reporter comprises a fluorescent protein. In some embodiments, the fluorescent protein is GFP, EBFP, EBFP2, Azurite, mKalamal, ECFP, Cerulean, CyPet, YFP, Citrine, Venus, YPet, RFP, CFP, or a derivative thereof. In some embodiments, the reporter is acetohydroxyacid synthase (AHAS), alkaline phosphatase (AP), beta galactosidase (LacZ), beta glucuronidase (GUS), chloramphenicol acetyltransferase (CAT), horseradish peroxidase (HRP), luciferase (Luc), nopaline synthase (NOS), octopine synthase (OCS), luciferase, or a derivative thereof. In some embodiments, the marker is an antibiotic resistance marker. In some embodiments, the antibiotic resistance marker is kanamycin, spectinomycin, streptomycin, ampicillin, carbenicillin, bleomycin, erythromycin, polymyxin B, tetracycline, chloramphenicol, neomycin, zeocin, or a derivative thereof. In some embodiments, the marker is a cell surface marker.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a multiple sequence alignment of MG178 family Large Serine Recombinase (LSR) candidates vs. a Bxb1 LSR reference sequence. Resolvase, recombinase, and Zn-finger domains are shown as boxes, and catalytic residues required for activity are highlighted as bars below each residue.

FIGS. 2A and 2B show a phylogenetic protein tree of LSRs of the disclosure. The tree was inferred from a global multiple sequence alignment of LSR sequences clustered at 90% amino acid identity (AAI). Selected MG178 family candidates are highlighted by large dots and are color-coded by the bacterial host that they target (FIG. 2A) or the host gene into which they insert (FIG. 2B).

FIG. 3 shows the analysis of an exemplary LSR integration site that was identified from alignments of genomic fragments with and without the prophage. The top panel shows a multiple sequence alignment of the genomic fragment with an integrated prophage (top) and its unintegrated host (bottom). Genes are predicted as arrows and functional domains supporting functional annotations are represented by black bars under genes. The prophage was predicted with CheckV (top) and integrates into a gene with a Quenosine biosynthesis protein annotation (bottom). The bottom panel shows a graph demonstrating that from the confirmation of prophage boundaries, the common core motif that is shared with the unintegrated host can be determined. The LSR gene is located on one of the prophage edges (black box).

FIGS. 4A-4C show a schematic of an exemplary in vitro screening procedure for serine recombinase recombination activity. FIG. 4A shows a schematic of recombinase in vitro expression from a linear or circular dsDNA construct. FIG. 4B shows a schematic for a recombination reaction using integrase that is added to the recombination reaction together with attP and attB dsDNA fragments specific to the serine recombinase. FIG. 4C shows a schematic of a PCR analysis by agarose gel electrophoresis of the recombined DNA amplified by attL- and attR-specific primers.

FIGS. 5A-5B show the results of in vitro recombinase assays for LSRs MG178-4, MG178-9, MG178-10, and MG178-11. Arrows indicate positive recombination event products. FIG. 5A shows the results of in vitro recombinase assays with AttL-specific primers used to amplify potential recombination events. Lane 1 shows the negative control for MG178-4 containing MG178-4 attB and MG178-4 attP dsDNA fragments. Lane 2 shows the experimental conditions for MG178-4 containing MG178-4 attB and MG178-4 attP dsDNA fragments and expressed MG178-4 recombinase. Lane 3 shows the negative control for MG178-9 containing MG178-9 attB and MG178-9 attP dsDNA fragments. Lane 4 shows the experimental conditions for MG178-9 containing MG178-9 attB and MG178-9 attP dsDNA fragments and expressed MG178-9 recombinase. Lane 5 shows the negative control for MG178-10 containing MG178-10 attB and MG178-10 attP dsDNA fragments. Lane 6 shows the experimental conditions for MG178-10 containing MG178-10 attB and MG178-10 attP dsDNA fragments and expressed MG178-10 recombinase. Lane 7 shows the negative control for MG178-11 containing MG178-11 attB and MG178-11 attP dsDNA fragments. Lane 8 shows the experimental conditions for MG178-11 containing MG178-11 attB and MG178-11 attP dsDNA fragments and expressed MG178-11 recombinase. FIG. 5B shows the results of in vitro recombinase assays with AttR-specific primers used to amplify potential recombination events. Lane 1 shows the negative control for MG178-4 containing MG178-4 attB and MG178-4 attP dsDNA fragments. Lane 2 shows the experimental conditions for MG178-4 containing MG178-4 attB and MG178-4 attP dsDNA fragments and expressed MG178-4 recombinase. Lane 3 shows the negative control for MG178-9 containing MG178-9 attB and MG178-9 attP dsDNA fragments. Lane 4 shows the experimental conditions for MG178-9 containing MG178-9 attB and MG178-9 attP dsDNA fragments and expressed MG178-9 recombinase. Lane 5 shows the negative control for MG178-10 containing MG178-10 attB and MG178-10 attP dsDNA fragments. Lane 6 shows the experimental conditions for MG178-10 containing MG178-10 attB and MG178-10 attP dsDNA fragments and expressed MG178-10 recombinase. Lane 7 shows the negative control for MG178-11 containing MG178-11 attB and MG178-11 attP dsDNA fragments. Lane 8 shows the experimental conditions for MG178-11 containing MG178-11 attB and MG178-11 attP dsDNA fragments and expressed MG178-11 recombinase.

FIG. 6 shows a schematic of an experimentally validated MG178-10 attL sequence that aligns with the bioinformatically identified MG178-10 attL. Black bars indicate 100% identity to the reference sequence (Found attL). The lower panel shows a zoomed sequence view of the alignment of reconstituted attP, reconstituted attB, and the experimentally determined attL site to the bioinformatically identified attL site for MG178-10. The grey highlighted sequence reflects the identity of the reconstructed attP and attB sites, and the lighter bases indicate discordant alignment from the reference sequence (bioinformatically identified attL). The boxed sequence is highlighting the conservation of the common core across found attL, attP, attB and sequenced attL. FIG. 6 discloses SEQ ID NOS 7435-7437 and 7435, respectively, in order of appearance.

FIG. 7 shows a schematic of an experimentally validated MG178-10 attR sequence aligned with the bioinformatically identified MG178-10 attR. Black bars indicate 100% identity to the reference sequence (Found attR). The lower panel shows a zoomed sequence view of the alignment of reconstituted attP, reconstituted attB, and the experimentally determined attR site to the bioinformatically identified attR site for MG178-10. The grey highlighted sequence reflects the identity of the reconstructed attP and attB site, and the lighter colored bases indicate discordant alignment from the reference sequence (bioinformatically identified attR). The boxed sequence is highlighting the conservation of the common core across found attR, attP, attB and sequenced attR. FIG. 7 discloses SEQ ID NOS 7438-7440 and 7438, respectively, in order of appearance.

FIG. 8 shows multiple sequence alignment of MG178 LSR candidates vs. a Bxb1 LSR reference sequence. Resolvase, recombinase, and Zn-finger domains are shown as boxes and catalytic residues required for activity are highlighted as bars below each residue.

FIGS. 9A-9C show pairwise alignments of the 3′ and 5′ regions flanking the proviruses of MG178-7202 (FIG. 9A), MG178-1859 (FIG. 9B), and MG178-7193 (FIG. 9C). Annotated are the provirus boundaries and common cores. Provirus boundaries were predicted and determined by aligning the provirus containing contigs to contigs lacking the provirus. The common cores were identified by finding conserved regions in the alignment. In cases where the alignment showed no conservation (FIG. 9C), repeats were identified within and outside of the provirus boundaries and the alignment was manually refined. FIG. 9A discloses SEQ ID NOS 7441-7443, FIG. 9B discloses SEQ ID NOS 7444-7446, and FIG. 9C discloses SEQ ID NOS 7447-7449, all respectively, in order of appearance.

FIGS. 10A-10B show the LSR-mediated attachment site recombination event and in cell plasmid recombination activity. FIG. 10A depicts a schematic illustration showing the LSR-mediated attachment site recombination event. FIG. 10B depicts a bar graph showing recombination activities. Active LSRs with recombination over 5% are plotted in comparison to BxB1 as reference. Each bar represents an experimental condition with a recombinase, AttB, and AttP plasmids transfected in HEK293T cells. Plasmid recombination was quantified by flow cytometry after 48 hours and percent recombination was calculated based on cells expressing both eGFP (recombinase protein) and mCherry (recombination event). Error bars are included for candidates with replicates.

FIG. 11 depicts the results of in vitro recombinase assays for LSR systems MG178-7202, MG178-1859, MG178-7193, MG178-7177. Lane 1 shows the ladder. Lane 2 shows the experimental conditions for MG178-7202 containing MG178-7202 attB and MG178-7202 attP dsDNA fragments, with addition of expressed MG178-7202 recombinase. Lane 3 shows the experimental conditions for MG178-1859 containing MG178-1859 attB and MG178-1859 attP dsDNA fragments, with addition of expressed MG178-1859 recombinase. Lane 4 shows the experimental conditions for MG178-7193 containing MG178-7193 attB and MG178-7193 attP dsDNA fragments, with addition of expressed MG178-7193 recombinase. Lane 5 shows the experimental conditions for MG178-7177 containing MG178-7177 attB and MG178-7177 attP dsDNA fragments, with addition of expressed MG178-7177 recombinase. Lane 6 shows the negative controls ladder. Lane 7 shows the negative control for MG178-7202 containing MG178-7202 attB and MG178-7202 attP dsDNA fragments but no enzyme. Lane 8 shows the negative control for MG178-1859 containing MG178-1859 attB and MG178-1859 attP dsDNA fragments but no enzyme. Lane 9 shows the negative control for MG178-7193 containing MG178-7193 attB and MG178-7193 attP dsDNA fragments but no enzyme. Lane 10 shows the negative control for MG178-7177 containing MG178-7177 attB and MG178-7177 attP dsDNA fragments but no enzyme.

FIG. 12 depicts a bar plot showing active candidates in human cells. Percent recombination was determined as the percentage of cells positive for mCherry (recombination) divided by the total number of cells positive for eGFP (integrase transfection and expression).

FIGS. 13A-13B depict plasmid dosage finds for optimal plasmid transfection concentrations in human cells. FIG. 13A depicts a bar plot showing percent recombination. FIG. 13B shows a table outlined the tested conditions. Optimal performance of MG178-7202 was found to be with equal weight integrase, attB, and attP plasmids at 250 ng per transfection for each.

FIG. 14 depicts a bar plot showing attachment site minimization for MG178-7202 (−47) in human cells. AttB sites were tested from 108 nt to 28 nt and AttP from 68 to 48 nt. Optimal conditions were determined to be 48 nt AttB and 58 nt AttP, while measurable recombination is able to be measured down to 32 nt of attB.

FIG. 15 depicts a bar plot showing attachment site minimization for MG178-7193 (−36) in human cells. AttB sites were tested from 72 to 52 nt and AttP from 72 to 52 nt. Optimal conditions were determined to be 52 nt AttB and 72 nt AttP.

FIGS. 16A-16C show the results of the purification and activity analyses of MG178-7202. Proteins expression induction and purification was monitored via SDS-PAGE (FIG. 16A). Expected protein MW was ˜76 kDa. Sumo-fused concentrated protein was run over an S200i 10 300 SEC column (FIG. 16B). Eluted fractions were visualized via SDS-PAGE (FIG. 16C) and fraction with purified protein were collected and concentrated (shaded area in FIG. 16B).

FIG. 17 depicts the results of in vitro recombinase assays for LSR MG178-7202, MG178-1859. Expected band for MG178-7202 and MG178-1859 are 1027 bp and 1167 bp respectively. Lane 1 shows the ladder for in vitro expressed proteins. Lane 2 shows the negative control for MG178-7202 containing MG178-7202 attB and MG178-7202 attP dsDNA fragments. Lane 3 shows the experimental conditions for MG178-7202 containing MG178-7202 attB and MG178-7202 attP dsDNA fragments and expressed MG178-7202 recombinase. Lane 4 shows the negative control for MG178-1859 containing MG178-1859 attB and MG178-1859 attP dsDNA fragments. Lane 5 shows the experimental conditions for MG178-1859 containing MG178-1859 attB and MG178-1859 attP dsDNA fragments and expressed MG178-1859 recombinase. Lane 6 shows the ladder for purified proteins. Lane 7 shows the negative control for MG178-7202 containing MG178-7202 attB and MG178-7202 attP dsDNA fragments. Lane 8 shows the experimental conditions for MG178-7202 containing MG178-7202 attB and MG178-7202 attP dsDNA fragments and purified MG178-7202 recombinase. Lane 9 shows the negative control for MG178-1859 containing MG178-1859 attB and MG178-1859 attP dsDNA fragments. Lane 10 shows the experimental conditions for MG178-1859 containing MG178-1859 attB and MG178-1859 attP dsDNA fragments and purified MG178-1859 recombinase.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

SEQ ID NOs: 1-16, 7151-7210, 7215-7218, 7220-7223, 7225-7230, 7233-7236, 7238-7241, 7243-7246, 7248-7251, 7253-7256, 7258-7261, 7263-7268, 7271-7274, 7277-7280, 7282-7285, 7287-7290, 7292-7295, 7297-7300, 7302-7305, 7307-7310, 7312-7315, 7317-7320, 7322-7325, 7327-7330, 7332-7335, 7337-7340, 7342-7345, 7347-7350, 7352-7355, 7357-7360, 7362-7365, 7367-7370, 7372-7375, 7377-7380, 7382-7385, 7387-7390, 7392-7395, 7397-7400, and 7402-7405 show nucleotide sequences of MG178 recombinase attachment sites.

SEQ ID NOs: 17-18, 7145, 7147, 7150, 7219, 7224, 7231, 7232, 7237, 7242, 7252, 7269, 7270, 7275, 7276, 7281, 7301, 7306, 7311, 7316, 7326, 7336, 7341, 7346, 7351, 7356, 7361, 7366, 7371, 7376, 7381, 7386, 7391, and 7401 show nucleotide sequences of MG178 conserved cores.

SEQ ID NOs: 21-7060, 7105-7142, and 7211-7214 show amino acid sequences of MG178 family large serine recombinases suitable for use in gene editing as described herein.

SEQ ID NOs: 7412-7415 and 7418 show amino acid sequences of MG178 recombinases protein tags.

SEQ ID NOs: 7407-7411 and 7416-7417 show nucleotide sequences of primers.

### DETAILED DESCRIPTION

Site-directed gene editing systems are powerful tools for site-directed genome engineering in cells. Most of the current gene editing systems depend on DNA double-stranded breaks (DSBs) to direct cellular DNA repair pathways such as homologous recombination (HR). However, these gene editing systems are often correlated with high indel rates, low insertion efficiency, high off-target activity, and a limited cargo size.

Additionally, the repair or insertion of longer pieces of DNA has remained challenging, and a safe and efficient way of targeted integration of large templates into a genome, for example for gene therapies or engineered cell therapies, is lacking. To date, lentiviruses or adeno-associated viruses (AAV) in combination with a CRISPR nuclease are used to insert large pieces of DNA, for example whole genes. However, lentiviral-mediated integration lacks the targetability feature, as integration occurs mostly randomly in open chromatin. AAV-mediated delivery has a limited cargo capacity and is not available for all cell types. A safe and efficient targeted genome editing system that allows for large template integration is needed.

The present disclosure is based, in part, upon the development of gene editing systems comprising large serine recombinases (LSRs) or serine recombinases for targetable and programmable integration of large fragments of DNA into a eukaryotic genome. In some embodiments, serine recombinases described herein can integrate multi-kilobase DNA sequences.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, refers to a base-sugar-phosphate combination. Contemplated nucleotides include naturally occurring nucleotides and synthetic nucleotides. Nucleotides are monomeric units of a nucleic acid sequence (e.g., deoxyribonucleic acid (DNA) and ribonucleic acid (RNA)). The term nucleotide includes ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives include, for example, [αS] dATP, 7-deaza-dGTP and 7-deaza-dATP, and nucleotide derivatives that confer nuclease resistance on the nucleic acid molecule containing them. The term nucleotide as used herein encompasses dideoxyribonucleoside triphosphates (ddNTPs) and their derivatives. Illustrative examples of ddNTPs include, but are not limited to, ddATP, ddCTP, ddGTP, ddITP, and ddTTP. A nucleotide may be unlabeled or detectably labeled, such as using moieties comprising optically detectable moieties (e.g., fluorophores) or quantum dots. Detectable labels include, for example, radioactive isotopes, fluorescent labels, chemiluminescent labels, bioluminescent labels, and enzyme labels. Fluorescent labels of nucleotides include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides include [R6G] dUTP, [TAMRA] dUTP, [R110] dCTP, [R6G] dCTP, [TAMRA] dCTP, [JOE] ddATP, [R6G] ddATP, [FAM] ddCTP, [R110] ddCTP, [TAMRA] ddGTP, [ROX] ddTTP, [dR6G] ddATP, [dR110] ddCTP, [dTAMRA] ddGTP, and [dROX] ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. The term nucleotide encompasses chemically modified nucleotides. An exemplary chemically-modified nucleotide is biotin-dNTP. Non-limiting examples of biotinylated dNTPs include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer is interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids. Modified amino acids include amino acids that have been chemically modified to include a group or a chemical moiety not naturally present on the amino acid. The term “amino acid” includes both D-amino acids and L-amino acids.

As used herein, the “non-native” refers to a nucleic acid or polypeptide sequence that is non-naturally occurring. Non-native refers to a non-naturally occurring nucleic acid or polypeptide sequence that comprises modifications such as mutations, insertions, or deletions. The term non-native encompasses fusion nucleic acids or polypeptides that encodes or exhibits an activity (e.g., enzymatic activity, methyltransferase activity, acetyltransferase activity, kinase activity, ubiquitinating activity, etc.) of the nucleic acid or polypeptide sequence to which the non-native sequence is fused. A non-native nucleic acid or polypeptide sequence includes those linked to a naturally-occurring nucleic acid or polypeptide sequence (or a variant thereof) by genetic engineering to generate a chimeric nucleic acid or polypeptide sequence encoding a chimeric nucleic acid or polypeptide.

The term “promoter”, as used herein, refers to the regulatory DNA region which controls transcription or expression of a polynucleotide (e.g., a gene) and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. Eukaryotic basal promoters typically, though not necessarily, contain a TATA-box and/or a CAAT box.

The term “expression”, as used herein, refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, the term expression includes splicing of the mRNA in a eukaryotic cell.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, refers to a macromolecule or association of macromolecules that comprises or associates with a polynucleotide and which mediates delivery of the polynucleotide to a cell. Examples of vectors include nucleic-based vectors (e.g., plasmids and viral vectors) and liposomes. An exemplary nucleic-acid based vector comprises genetic elements, e.g., regulatory elements, operatively linked to a gene to facilitate expression of the gene in a target.

As used herein, “expression cassette” and “nucleic acid cassette” are used interchangeably to refer to a component of a vector comprising a combination of nucleic acid sequences or elements (e.g., therapeutic gene, promoter, and a terminator) that are expressed together or are operably linked for expression. The terms encompass an expression cassette including a combination of regulatory elements and a gene or genes to which they are operably linked for expression.

A “functional fragment” of a DNA or protein sequence refers to a fragment that retains a biological activity (either functional or structural) that is substantially similar to a biological activity of the full-length DNA or protein sequence. A biological activity of a DNA sequence includes its ability to influence expression in a manner attributed to the full-length sequence.

The terms “engineered,” “synthetic,” and “artificial” are used interchangeably herein to refer to an object that has been modified by human intervention. For example, the terms refer to a polynucleotide or polypeptide that is non-naturally occurring. An engineered peptide has, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. Non-limiting examples include the following: a nucleic acid modified by changing its sequence to a sequence that does not occur in nature; a nucleic acid modified by ligating it to a nucleic acid that it does not associate with in nature such that the ligated product possesses a function not present in the original nucleic acid; an engineered nucleic acid synthesized in vitro with a sequence that does not exist in nature; a protein modified by changing its amino acid sequence to a sequence that does not exist in nature; an engineered protein acquiring a new function or property. An “engineered” system comprises at least one engineered component.

As used herein, a “guide nucleic acid” or “guide polynucleotide” refers to a nucleic acid that may hybridize to a target nucleic acid and thereby directs an associated nuclease to the target nucleic acid. A guide nucleic acid is, but is not limited to, RNA (guide RNA or gRNA), DNA, or a mixture of RNA and DNA. A guide nucleic acid can include a crRNA or a tracrRNA or a combination of both. The term guide nucleic acid encompasses an engineered guide nucleic acid and a programmable guide nucleic acid to specifically bind to the target nucleic acid. A portion of the target nucleic acid may be complementary to a portion of the guide nucleic acid. The strand of a double-stranded target polynucleotide that is complementary to and hybridizes with the guide nucleic acid is the complementary strand. The strand of the double-stranded target polynucleotide that is complementary to the complementary strand, and therefore is not complementary to the guide nucleic acid is called noncomplementary strand. A guide nucleic acid having a polynucleotide chain is a “single guide nucleic acid.” A guide nucleic acid having two polynucleotide chains is a “double guide nucleic acid.” If not otherwise specified, the term “guide nucleic acid” is inclusive, referring to both single guide nucleic acids and double guide nucleic acids. A guide nucleic acid may comprise a segment referred to as a “nucleic acid-targeting segment” or a “nucleic acid-targeting sequence,” or a “spacer.” A nucleic acid-targeting segment can include a sub-segment referred to as a “protein binding segment” or “protein binding sequence” or “Cas protein binding segment.”

The term “tracrRNA” or “tracr sequence” means trans-activating CRISPR RNA. tracrRNA interacts with the CRISPR (cr) RNA to form a guide nucleic acid (e.g., guide RNA or gRNA) that may hybridize to a target nucleic acid and thereby directs an associated nuclease to the target nucleic acid.

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “transposon” refers to mobile elements that move in and out of genomes carrying “cargo DNA” with them. These transposons can differ on the type of nucleic acid to transpose, the type of repeat at the ends of the transposon, the type of cargo to be carried, or by the mode of transposition (i.e., self-repair or host-repair).

As used herein, the term “transposase” or “transposases” refers to an enzyme that binds to the end of a transposon and catalyzes its movement to another part of the genome. Types of movement include a cut and paste mechanism and a replicative transposition mechanism.

As used herein, the term “Tn7” or “Tn7-like transposase” refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site,” attTn7. TnsD is a member of a large family of proteins that also includes TniQ. TniQ has been shown to target transposition into resolution sites of plasmids.

As used herein, the terms “gene editing” and “genome editing” can be used interchangeably. Gene editing or genome editing means to change the nucleic acid sequence of a gene or a genome. Genome editing can include, for example, insertions, deletions, and mutations. Genome editing can be performed by a gene editing system, for example a nuclease, a reverse transcriptase, a recombinase, or a base editor.

As used herein, the term “recombinase” refers to an enzyme that mediates the recombination of DNA fragments located between recombinase recognition sequences, which results in the excision, insertion, inversion, exchange or translocation) of the DNA fragments located between the recombinase recognition sequences.

As used herein, the term “recombine,” or “recombination,” in the context of a nucleic acid modification (e.g., a genomic modification), refers to the process by which two or more nucleic acid molecules, or two or more regions of a single nucleic acid molecule, are modified by the action of a recombinase protein. Recombination can result in, inter alia, the insertion, inversion, excision, or translocation of a nucleic acid sequence, e.g., in or between one or more nucleic acid molecules.

As used herein, the term “complex” refers to a joining of at least two components. The two components may each retain the properties/activities they had prior to forming the complex or gain properties as a result of forming the complex. The joining includes, but is not limited to, covalent bonding, non-covalent bonding (i.e., hydrogen bonding, ionic interactions, Van der Waals interactions, and hydrophobic bond), use of a linker, fusion, or any other suitable method. Contemplated components of the complex include polynucleotides, polypeptides, or combinations thereof. For example, a complex comprises an endonuclease and a guide polynucleotide.

The term“contig” or “contigs” is a set of DNA segments or sequences that overlap in a way that provides a contiguous representation of a genomic region.

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of the large serine recombinase protein sequences described herein (e.g., MG178 family large serine recombinase, or any other family large serine recombinase described herein). In some embodiments, such conservatively substituted variants are functional variants. Such functional variants can encompass sequences with substitutions such that the activity of one or more critical active site residues are not disrupted.

Also included in the current disclosure are variants of any of the enzymes described herein with substitution of one or more catalytic residues to decrease or eliminate activity of the enzyme (e.g. decreased-activity variants). In some embodiments, a decreased activity variant of a protein described herein comprises a disrupting substitution of at least one, at least two, or all three catalytic residues.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

### Serine Recombinase Gene Editing Systems

Current gene editing systems lack the ability to integrate multi-kilobase nucleic acid sequences. Several of these gene editing systems primarily rely on nuclease-directed DNA double-stranded breaks (DSBs) to direct cellular DNA repair pathways, such as homologous recombination (HR). Despite important advances in optimizing HR in specific contexts, these approaches generally suffer from low insertion efficiency, high indel rates and cargo size limitations, with limited success for cargoes larger than 1 kilobase (kb).

Large serine recombinases (LSRs) are capable of integrating large fragments of DNA into a eukaryotic genome in a non-random, site-specific manner. Viral LSRs range between 400 and 700 amino acids long and drive phage genome integration into a bacterial host genome when the virus enters its lysogenic life cycle. The mechanism for prophage integration involves the LSR recognizing a specific attachment site in the host genome, the attB site, and a phage attachment site, the attP site, on the phage genome. Viral genome integration occurs via recombination at these attachment sites, a process that leads to the generation of two new attachment sites, the attL and attR sites flanking the prophage.

By recognizing these attachment sites (i.e. recognition sequences found on DNA donor and acceptor molecules), recombinases are capable of catalyzing target cleavage, strand exchange and DNA rejoining. This mechanism enables site-specific DNA insertion without requiring any cellular cofactors and without generating exposed double strand breaks. However, several LSRs suffer from limited efficiency in DNA integration. As such, improved LSRs are needed.

Serine recombinases described herein provided for genome engineering due to their ability to integrate a desired cargo into a specific target site.

### Serine Recombinases

Described herein are gene editing systems comprising: a serine recombinase comprising at least about 80% sequence identity to any one of SEQ ID NOs: 21-7060 and 7105-7142 or a nucleic acid encoding the serine recombinase. Further described herein are nucleic acids, vectors, and cells comprising a serine recombinase described herein. Further described herein are means for integrating nucleic acid sequences in a genome.

Serine recombinases are enzymes that catalyze site-specific recombination events by facilitating DNA strand exchanges between two DNA segments possessing cognate recombinase recognition sites. The serine recombinase family comprises, for example, the small serine recombinases gamma-delta resolvase (from the Tn1000 transposon) and Tn3 resolvase (from the Tn3 transposon), or the large serine recombinases (LSRs) φC31-integrase (from the φC31 phage), Bxb1-integrase (from the mycobacteriophage), and R4 integrase. Serine recombinases are characterized by a conserved catalytic serine amino acid residue that attacks the DNA phosphodiester and becomes covalently linked to a DNA strand end during catalysis. Serine recombinases recognize cognate attachment site sequences termed attB on the acceptor DNA strand (for example a bacterial genome) and attP on the donor DNA strand (for example the phage genome). After the recombination event, the attB and attP sites are recombined to form the attL and attR sites flanking the newly integrated sequence. attB and attP sites are typically up to about 50 bases long. During the recombination event, the serine recombinases form a tetrameric complex, with a protein dimer each attaching to an attB or attP attachment site. The serine recombinases cleave each strand producing a double strand break and leaving a 2 bp overhang and then strand exchange and ligate the strands. Typically, for serine recombinases, no other enzymes are needed to perform the reaction.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of SEQ ID NOs: 21-7060 and 7105-7142. In some embodiments, the serine recombinase comprises a sequence having at least about 70% identity to any one of SEQ ID NOs: 21-7060 and 7105-7142. In some embodiments, the serine recombinase comprises a sequence having at least about 75% identity to any one of SEQ ID NOs: 21-7060 and 7105-7142. In some embodiments, the serine recombinase comprises a sequence having at least about 80% identity to any one of SEQ ID NOs: 21-7060 and 7105-7142. In some embodiments, the serine recombinase comprises a sequence having at least about 85% identity to any one of SEQ ID NOs: 21-7060 and 7105-7142. In some embodiments, the serine recombinase comprises a sequence having at least about 90% identity to any one of SEQ ID NOs: 21-7060 and 7105-7142. In some embodiments, the serine recombinase comprises a sequence having at least about 95% identity to any one of SEQ ID NOs: 21-7060 and 7105-7142. In some embodiments, the serine recombinase comprises a sequence having at least about 96% identity to any one of SEQ ID NOs: 21-7060 and 7105-7142. In some embodiments, the serine recombinase comprises a sequence having at least about 97% identity to any one of SEQ ID NOs: 21-7060 and 7105-7142. In some embodiments, the serine recombinase comprises a sequence having at least about 98% identity to any one of SEQ ID NOs: 21-7060 and 7105-7142. In some embodiments, the serine recombinase comprises a sequence having at least about 99% identity to any one of SEQ ID NOs: 21-7060 and 7105-7142. In some embodiments, the serine recombinase comprises a sequence having 100% identity to any one of SEQ ID NOs: 21-7060 and 7105-7142.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 21. In some embodiments, the serine recombinase comprises a sequence having at least about 70% identity to SEQ ID NO: 21. In some embodiments, the serine recombinase comprises a sequence having at least about 75% identity to SEQ ID NO: 21. In some embodiments, the serine recombinase comprises a sequence having at least about 80% identity to SEQ ID NO: 21. In some embodiments, the serine recombinase comprises a sequence having at least about 85% identity to SEQ ID NO: 21. In some embodiments, the serine recombinase comprises a sequence having at least about 90% identity to SEQ ID NO: 21. In some embodiments, the serine recombinase comprises a sequence having at least about 95% identity to SEQ ID NO: 21. In some embodiments, the serine recombinase comprises a sequence having at least about 96% identity to SEQ ID NO: 21. In some embodiments, the serine recombinase comprises a sequence having at least about 97% identity to SEQ ID NO: 21. In some embodiments, the serine recombinase comprises a sequence having at least about 98% identity to SEQ ID NO: 21. In some embodiments, the serine recombinase comprises a sequence having at least about 99% identity to SEQ ID NO: 21. In some embodiments, the serine recombinase comprises a sequence having 100% identity to SEQ ID NO: 21.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 22. In some embodiments, the serine recombinase comprises a sequence having at least about 70% identity to SEQ ID NO: 22. In some embodiments, the serine recombinase comprises a sequence having at least about 75% identity to SEQ ID NO: 22. In some embodiments, the serine recombinase comprises a sequence having at least about 80% identity to SEQ ID NO: 22. In some embodiments, the serine recombinase comprises a sequence having at least about 85% identity to SEQ ID NO: 22. In some embodiments, the serine recombinase comprises a sequence having at least about 90% identity to SEQ ID NO: 22. In some embodiments, the serine recombinase comprises a sequence having at least about 95% identity to SEQ ID NO: 22. In some embodiments, the serine recombinase comprises a sequence having at least about 96% identity to SEQ ID NO: 22. In some embodiments, the serine recombinase comprises a sequence having at least about 97% identity to SEQ ID NO: 22. In some embodiments, the serine recombinase comprises a sequence having at least about 98% identity to SEQ ID NO: 22. In some embodiments, the serine recombinase comprises a sequence having at least about 99% identity to SEQ ID NO: 22. In some embodiments, the serine recombinase comprises a sequence having 100% identity to SEQ ID NO: 22.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 23. In some embodiments, the serine recombinase comprises a sequence having at least about 70% identity to SEQ ID NO: 23. In some embodiments, the serine recombinase comprises a sequence having at least about 75% identity to SEQ ID NO: 23. In some embodiments, the serine recombinase comprises a sequence having at least about 80% identity to SEQ ID NO: 23. In some embodiments, the serine recombinase comprises a sequence having at least about 85% identity to SEQ ID NO: 23. In some embodiments, the serine recombinase comprises a sequence having at least about 90% identity to SEQ ID NO: 23. In some embodiments, the serine recombinase comprises a sequence having at least about 95% identity to SEQ ID NO: 23. In some embodiments, the serine recombinase comprises a sequence having at least about 96% identity to SEQ ID NO: 23. In some embodiments, the serine recombinase comprises a sequence having at least about 97% identity to SEQ ID NO: 23. In some embodiments, the serine recombinase comprises a sequence having at least about 98% identity to SEQ ID NO: 23. In some embodiments, the serine recombinase comprises a sequence having at least about 99% identity to SEQ ID NO: 23. In some embodiments, the serine recombinase comprises a sequence having 100% identity to SEQ ID NO: 23.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 24. In some embodiments, the serine recombinase comprises a sequence having at least about 70% identity to SEQ ID NO: 24. In some embodiments, the serine recombinase comprises a sequence having at least about 75% identity to SEQ ID NO: 24. In some embodiments, the serine recombinase comprises a sequence having at least about 80% identity to SEQ ID NO: 24. In some embodiments, the serine recombinase comprises a sequence having at least about 85% identity to SEQ ID NO: 24. In some embodiments, the serine recombinase comprises a sequence having at least about 90% identity to SEQ ID NO: 24. In some embodiments, the serine recombinase comprises a sequence having at least about 95% identity to SEQ ID NO: 24. In some embodiments, the serine recombinase comprises a sequence having at least about 96% identity to SEQ ID NO: 24. In some embodiments, the serine recombinase comprises a sequence having at least about 97% identity to SEQ ID NO: 24. In some embodiments, the serine recombinase comprises a sequence having at least about 98% identity to SEQ ID NO: 24. In some embodiments, the serine recombinase comprises a sequence having at least about 99% identity to SEQ ID NO: 24. In some embodiments, the serine recombinase comprises a sequence having 100% identity to SEQ ID NO: 24.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7140. In some embodiments, the serine recombinase comprises a sequence having at least about 70% identity to SEQ ID NO: 7140. In some embodiments, the serine recombinase comprises a sequence having at least about 75% identity to SEQ ID NO: 7140. In some embodiments, the serine recombinase comprises a sequence having at least about 80% identity to SEQ ID NO: 7140. In some embodiments, the serine recombinase comprises a sequence having at least about 85% identity to SEQ ID NO: 7140. In some embodiments, the serine recombinase comprises a sequence having at least about 90% identity to SEQ ID NO: 7140. In some embodiments, the serine recombinase comprises a sequence having at least about 95% identity to SEQ ID NO: 7140. In some embodiments, the serine recombinase comprises a sequence having at least about 96% identity to SEQ ID NO: 7140. In some embodiments, the serine recombinase comprises a sequence having at least about 97% identity to SEQ ID NO: 7140. In some embodiments, the serine recombinase comprises a sequence having at least about 98% identity to SEQ ID NO: 7140. In some embodiments, the serine recombinase comprises a sequence having at least about 99% identity to SEQ ID NO: 7140. In some embodiments, the serine recombinase comprises a sequence having 100% identity to SEQ ID NO: 7140.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7131. In some embodiments, the serine recombinase comprises a sequence having at least about 70% identity to SEQ ID NO: 7131. In some embodiments, the serine recombinase comprises a sequence having at least about 75% identity to SEQ ID NO: 7131. In some embodiments, the serine recombinase comprises a sequence having at least about 80% identity to SEQ ID NO: 7131. In some embodiments, the serine recombinase comprises a sequence having at least about 85% identity to SEQ ID NO: 7131. In some embodiments, the serine recombinase comprises a sequence having at least about 90% identity to SEQ ID NO: 7131. In some embodiments, the serine recombinase comprises a sequence having at least about 95% identity to SEQ ID NO: 7131. In some embodiments, the serine recombinase comprises a sequence having at least about 96% identity to SEQ ID NO: 7131. In some embodiments, the serine recombinase comprises a sequence having at least about 97% identity to SEQ ID NO: 7131. In some embodiments, the serine recombinase comprises a sequence having at least about 98% identity to SEQ ID NO: 7131. In some embodiments, the serine recombinase comprises a sequence having at least about 99% identity to SEQ ID NO: 7131. In some embodiments, the serine recombinase comprises a sequence having 100% identity to SEQ ID NO: 7131.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7115. In some embodiments, the serine recombinase comprises a sequence having at least about 70% identity to SEQ ID NO: 7115. In some embodiments, the serine recombinase comprises a sequence having at least about 75% identity to SEQ ID NO: 7115. In some embodiments, the serine recombinase comprises a sequence having at least about 80% identity to SEQ ID NO: 7115. In some embodiments, the serine recombinase comprises a sequence having at least about 85% identity to SEQ ID NO: 7115. In some embodiments, the serine recombinase comprises a sequence having at least about 90% identity to SEQ ID NO: 7115. In some embodiments, the serine recombinase comprises a sequence having at least about 95% identity to SEQ ID NO: 7115. In some embodiments, the serine recombinase comprises a sequence having at least about 96% identity to SEQ ID NO: 7115. In some embodiments, the serine recombinase comprises a sequence having at least about 97% identity to SEQ ID NO: 7115. In some embodiments, the serine recombinase comprises a sequence having at least about 98% identity to SEQ ID NO: 7115. In some embodiments, the serine recombinase comprises a sequence having at least about 99% identity to SEQ ID NO: 7115. In some embodiments, the serine recombinase comprises a sequence having 100% identity to SEQ ID NO: 7115.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7139. In some embodiments, the serine recombinase comprises a sequence having at least about 70% identity to SEQ ID NO: 7139. In some embodiments, the serine recombinase comprises a sequence having at least about 75% identity to SEQ ID NO: 7139. In some embodiments, the serine recombinase comprises a sequence having at least about 80% identity to SEQ ID NO: 7139. In some embodiments, the serine recombinase comprises a sequence having at least about 85% identity to SEQ ID NO: 7139. In some embodiments, the serine recombinase comprises a sequence having at least about 90% identity to SEQ ID NO: 7139. In some embodiments, the serine recombinase comprises a sequence having at least about 95% identity to SEQ ID NO: 7139. In some embodiments, the serine recombinase comprises a sequence having at least about 96% identity to SEQ ID NO: 7139. In some embodiments, the serine recombinase comprises a sequence having at least about 97% identity to SEQ ID NO: 7139. In some embodiments, the serine recombinase comprises a sequence having at least about 98% identity to SEQ ID NO: 7139. In some embodiments, the serine recombinase comprises a sequence having at least about 99% identity to SEQ ID NO: 7139. In some embodiments, the serine recombinase comprises a sequence having 100% identity to SEQ ID NO: 7139.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1848. In some embodiments, the serine recombinase comprises a sequence having at least about 70% identity to SEQ ID NO: 1848. In some embodiments, the serine recombinase comprises a sequence having at least about 75% identity to SEQ ID NO: 1848. In some embodiments, the serine recombinase comprises a sequence having at least about 80% identity to SEQ ID NO: 1848. In some embodiments, the serine recombinase comprises a sequence having at least about 85% identity to SEQ ID NO: 1848. In some embodiments, the serine recombinase comprises a sequence having at least about 90% identity to SEQ ID NO: 1848. In some embodiments, the serine recombinase comprises a sequence having at least about 95% identity to SEQ ID NO: 1848. In some embodiments, the serine recombinase comprises a sequence having at least about 96% identity to SEQ ID NO: 1848. In some embodiments, the serine recombinase comprises a sequence having at least about 97% identity to SEQ ID NO: 1848. In some embodiments, the serine recombinase comprises a sequence having at least about 98% identity to SEQ ID NO: 1848. In some embodiments, the serine recombinase comprises a sequence having at least about 99% identity to SEQ ID NO: 1848. In some embodiments, the serine recombinase comprises a sequence having 100% identity to SEQ ID NO: 1848.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7111. In some embodiments, the serine recombinase comprises a sequence having at least about 70% identity to SEQ ID NO: 7111. In some embodiments, the serine recombinase comprises a sequence having at least about 75% identity to SEQ ID NO: 7111. In some embodiments, the serine recombinase comprises a sequence having at least about 80% identity to SEQ ID NO: 7111. In some embodiments, the serine recombinase comprises a sequence having at least about 85% identity to SEQ ID NO: 7111. In some embodiments, the serine recombinase comprises a sequence having at least about 90% identity to SEQ ID NO: 7111. In some embodiments, the serine recombinase comprises a sequence having at least about 95% identity to SEQ ID NO: 7111. In some embodiments, the serine recombinase comprises a sequence having at least about 96% identity to SEQ ID NO: 7111. In some embodiments, the serine recombinase comprises a sequence having at least about 97% identity to SEQ ID NO: 7111. In some embodiments, the serine recombinase comprises a sequence having at least about 98% identity to SEQ ID NO: 7111. In some embodiments, the serine recombinase comprises a sequence having at least about 99% identity to SEQ ID NO: 7111. In some embodiments, the serine recombinase comprises a sequence having 100% identity to SEQ ID NO: 7111.

In some embodiments, the serine recombinase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 7136. In some embodiments, the serine recombinase comprises a sequence having at least about 70% identity to SEQ ID NO: 7136. In some embodiments, the serine recombinase comprises a sequence having at least about 75% identity to SEQ ID NO: 7136. In some embodiments, the serine recombinase comprises a sequence having at least about 80% identity to SEQ ID NO: 7136. In some embodiments, the serine recombinase comprises a sequence having at least about 85% identity to SEQ ID NO: 7136. In some embodiments, the serine recombinase comprises a sequence having at least about 90% identity to SEQ ID NO: 7136. In some embodiments, the serine recombinase comprises a sequence having at least about 95% identity to SEQ ID NO: 7136. In some embodiments, the serine recombinase comprises a sequence having at least about 96% identity to SEQ ID NO: 7136. In some embodiments, the serine recombinase comprises a sequence having at least about 97% identity to SEQ ID NO: 7136. In some embodiments, the serine recombinase comprises a sequence having at least about 98% identity to SEQ ID NO: 7136. In some embodiments, the serine recombinase comprises a sequence having at least about 99% identity to SEQ ID NO: 7136. In some embodiments, the serine recombinase comprises a sequence having 100% identity to SEQ ID NO: 7136.

Further described herein are eukaryotic cells comprising a serine recombinase comprising at least about 80% sequence identity to any one of SEQ ID NOs: 21-7060 and 7105-7142. Further described herein are eukaryotic cells comprising a serine recombinase comprising at least about 80% sequence identity to SEQ ID NO: 21. Further described herein are eukaryotic cells comprising a serine recombinase comprises at least about 80% sequence identity to SEQ ID NO: 22. Further described herein are eukaryotic cells comprising a serine recombinase comprises at least about 80% sequence identity to SEQ ID NO: 23. Further described herein are eukaryotic cells comprising a serine recombinase comprises at least about 80% sequence identity to SEQ ID NO: 24.

In some embodiments, the eukaryotic cell is a mammalian cell. In some embodiments, the eukaryotic cell is a human cell.

In some embodiments, the serine recombinases described herein comprise improved integration efficiency. In some embodiments, the serine recombinases described herein comprise an integration efficiency of at least about 5%. In some embodiments, the serine recombinases described herein comprise an integration efficiency of at least about 25%. In some embodiments, the serine recombinases described herein comprise an integration efficiency of at least about 50%. In some embodiments, the serine recombinases described herein comprise an integration efficiency of at least about 5%, 10%, 15%, 20%, 25%, 30%, 35%, 40%, 45%, 50%, 55%, 60%, 65%, 70%, 75%, 80%, 85%, 90%, 95%, or more than 95%. In some embodiments, the serine recombinases described herein comprise an improved integration efficiency as compared to a serine recombinase selected from the group consisting of: β-six, CinH, ParA γδ, Bxb1, φC31, TP901, TG1, φBT1, R4, φRV1, φFC1, MR11, A118, U153, and gp29.

In some embodiments, the serine recombinase is a viral, prokaryotic, or eukaryotic serine recombinase. In some embodiments, the serine recombinase is capable of targeting genes comprising a catalase domain or synthase domain. In some embodiments, the catalase is manganese catalase. In some embodiments, the synthase is Queuosine synthase. In some embodiments, the serine recombinase is capable of targeting genes comprising a DUF4244 Pfam domain.

In some embodiments, the serine recombinase described herein comprises one or more nuclear localization sequences (NLSs) proximal to an N- or C-terminus of serine recombinase. In some embodiments, the NLS comprises any of the sequences in Table 1 below, or a combination thereof:

TABLE 1
Example NLS Sequences

| Source | NLS amino acid sequence | SEQ ID NO: |
|---|---|---|
| SV40 | PKKKRKV | 7419 |
| nucleoplasmin bipartite NLS | KRPAATKKAGQAKKKK | 7420 |
| c-myc NLS | PAAKRVKLD | 7421 |
| c-myc NLS | RQRRNELKRSP | 7422 |
| hRNPA1 M9 NLS | NQSSNFGPMKGGNFGGRSSGPYGGGGQYFA | 7423 |
| | KPRNQGGY | |
| Importin-alpha IBB domain | RMRIZFKNKGKDTAELRRRRVEVSVELRKAK | 7424 |
| | KDEQILKRRNV | |
| Myoma T protein | VSRKRPRP | 7425 |
| Myoma T protein | PPKKARED | 7426 |
| p53 | PQPKKKPL | 7427 |
| mouse c-abl IV | SALIKKKKKMAP | 7428 |
| influenza virus NS1 | DRLRR | 7429 |
| influenza virus NS1 | PKQKKRK | 7430 |
| Hepatitis virus delta antigen | RKLKKKIKKL | 7431 |
| mouse Mx1 protein | REKKKFLKRR | 7432 |
| human poly(ADP-ribose) | KRKGDEVDGVDEVAKKKSKK | 7433 |
| polymerase | | |
| steroid hormone receptor | RKCLQAGMNLEARKTKK | 7434 |
| (human) glucocorticoid | | |

In some embodiments, the serine recombinase comprises a tag. In some embodiments, the tag is an affinity tag. Exemplary affinity tags include, but are not limited to, a His-tag, a Flag tag, a Myc-tag, an MBP-tag, and a GST-tag.

In some embodiments, the serine recombinase comprises a protease cleavage site. Exemplary protease cleavage sites include, but are not limited to, a TEV site, a C3 site, a Factor Xa site, and an Enterokinase site.

### Recombination Sites

Described herein are gene editing systems comprising: a serine recombinase comprising at least about 80% sequence identity to any one of SEQ ID NOs: 21-7060 and 7105-7142 or a nucleic acid encoding the serine recombinase; and a nucleic acid comprising a donor polynucleotide and a first attachment site sequence.

In some embodiments, the first attachment site sequence is 5′ of the donor polynucleotide.

In some embodiments, the nucleic acid encoding the serine recombinase further comprises a second attachment site sequence. In some embodiments, the second attachment site sequence is 5′ of the serine recombinase. In some embodiments, the nucleic acid encoding the serine recombinase comprises one or more attachment site sequences. In some embodiments, the nucleic acid encoding the serine recombinase comprises 1, 2, 3, 4, 5, or more than 5 attachment site sequences.

In some embodiments, the nucleic acid comprising a donor polynucleotide comprises one or more attachment site sequences. In some embodiments, the nucleic acid comprising a donor polynucleotide comprises 1, 2, 3, 4, 5, or more than 5 attachment site sequences.

In some embodiments, the first attachment site sequence and the second attachment site sequence are capable of recombination.

In some embodiments, the first attachment site sequence is a bacterial genomic recombination sequence (attB). In some embodiments, the attB sequence comprises about 20 to about 500 nucleotides. In some embodiments, the attB sequence comprises about 20 to about 450, about 20 to about 400, about 20 to about 350, about 20 to about 300, about 20 to about 250, about 20 to about 200, about 20 to about 250, about 20 to about 100, about 20 to about 50, about 50 to about 450, about 50 to about 400, about 50 to about 350, about 50 to about 300, about 50 to about 250, about 50 to about 200, about 50 to about 150, about 50 to about 100, about 100 to about 450, about 100 to about 400, about 100 to about 350, about 100 to about 300, about 100 to about 250, about 100 to about 200, or about 100 to about 150 nucleotides.

In some embodiments, the first attachment site sequence is a phage genomic recombination sequence (attP). In some embodiments, the attP sequence comprises about 20 to about 450, about 20 to about 400, about 20 to about 350, about 20 to about 300, about 20 to about 250, about 20 to about 200, about 20 to about 250, about 20 to about 100, about 20 to about 50, about 50 to about 450, about 50 to about 400, about 50 to about 350, about 50 to about 300, about 50 to about 250, about 50 to about 200, about 50 to about 150, about 50 to about 100, about 100 to about 450, about 100 to about 400, about 100 to about 350, about 100 to about 300, about 100 to about 250, about 100 to about 200, or about 100 to about 150 nucleotides.

In some embodiments, the second attachment site sequence is a bacterial genomic recombination sequence (attB). In some embodiments, the attB sequence comprises about 20 to about 500 nucleotides. In some embodiments, the attB sequence comprises about 20 to about 450, about 20 to about 400, about 20 to about 350, about 20 to about 300, about 20 to about 250, about 20 to about 200, about 20 to about 250, about 20 to about 100, about 20 to about 50, about 50 to about 450, about 50 to about 400, about 50 to about 350, about 50 to about 300, about 50 to about 250, about 50 to about 200, about 50 to about 150, about 50 to about 100, about 100 to about 450, about 100 to about 400, about 100 to about 350, about 100 to about 300, about 100 to about 250, about 100 to about 200, or about 100 to about 150 nucleotides.

In some embodiments, the second attachment site sequence is a phage genomic recombination sequence (attP). In some embodiments, the attP sequence comprises about 20 to about 450, about 20 to about 400, about 20 to about 350, about 20 to about 300, about 20 to about 250, about 20 to about 200, about 20 to about 250, about 20 to about 100, about 20 to about 50, about 50 to about 450, about 50 to about 400, about 50 to about 350, about 50 to about 300, about 50 to about 250, about 50 to about 200, about 50 to about 150, about 50 to about 100, about 100 to about 450, about 100 to about 400, about 100 to about 350, about 100 to about 300, about 100 to about 250, about 100 to about 200, or about 100 to about 150 nucleotides.

In some embodiments, the attB sequence comprises at least 70% (e.g., 75%, 80%, 90%, 95%, 97%, 98%, or 99%) sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least 75% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least 90% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least 95% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least 97% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least 98% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises at least 99% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402. In some embodiments, the attB sequence comprises any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402.

In some embodiments, the attB sequence comprises at least 70% (e.g., 75%, 80%, 90%, 95%, 97%, 98%, or 99%) sequence identity to any one of SEQ ID NOs: 1, 5, 9, and 13. In some embodiments, the attB sequence comprises at least 75% sequence identity to any one of SEQ ID NOs: 1, 5, 9, and 13. In some embodiments, the attB sequence comprises at least 80% sequence identity to any one of SEQ ID NOs: 1, 5, 9, and 13. In some embodiments, the attB sequence comprises at least 90% sequence identity to any one of SEQ ID NOs: 1, 5, 9, and 13. In some embodiments, the attB sequence comprises at least 95% sequence identity to any one of SEQ ID NOs: 1, 5, 9, and 13. In some embodiments, the attB sequence comprises at least 97% sequence identity to any one of SEQ ID NOs: 1, 5, 9, and 13. In some embodiments, the attB sequence comprises at least 98% sequence identity to any one of SEQ ID NOs: 1, 5, 9, and 13. In some embodiments, the attB sequence comprises at least 99% sequence identity to any one of SEQ ID NOs: 1, 5, 9, and 13. In some embodiments, the attB sequence comprises any one of SEQ ID NOs: 1, 5, 9, and 13.

In some embodiments, the attP sequence comprises at least 70% (e.g., 75%, 80%, 90%, 95%, 97%, 98%, or 99%) sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least 75% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least 80% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least 90% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least 95% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least 97% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least 98% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises at least 99% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404. In some embodiments, the attP sequence comprises any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404.

In some embodiments, the attP sequence comprises at least 70% (e.g., 75%, 80%, 90%, 95%, 97%, 98%, or 99%) sequence identity to any one of SEQ ID NOs: 2, 6, 10, and 14. In some embodiments, the attP sequence comprises at least 75% sequence identity to any one of SEQ ID NOs: 2, 6, 10, and 14. In some embodiments, the attP sequence comprises at least 80% sequence identity to any one of SEQ ID NOs: 2, 6, 10, and 14. In some embodiments, the attP sequence comprises at least 90% sequence identity to any one of SEQ ID NOs: 2, 6, 10, and 14. In some embodiments, the attP sequence comprises at least 95% sequence identity to any one of SEQ ID NOs: 2, 6, 10, and 14. In some embodiments, the attP sequence comprises at least 97% sequence identity to any one of SEQ ID NOs: 2, 6, 10, and 14. In some embodiments, the attP sequence comprises at least 98% sequence identity to any one of SEQ ID NOs: 2, 6, 10, and 14. In some embodiments, the attP sequence comprises at least 99% sequence identity to any one of SEQ ID NOs: 2, 6, 10, and 14. In some embodiments, the attP sequence comprises any one of SEQ ID NOs: 2, 6, 10, and 14.

In some embodiments, the nucleic acid comprising a donor polynucleotide and a first attachment site sequence are delivered by a plasmid, a nanoplasmid, a phagemid, a phage derivative, a virus, a bacmid, a bacterial artificial chromosome (BAC), a minicircle, a doggybone, a yeast artificial chromosome (YAC), or a cosmid.

Described herein are eukaryotic genomes comprising a donor polynucleotide sequence; and an attL sequence 5′ to the donor polynucleotide sequence, wherein the attL sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC. In some embodiments, the eukaryotic genomes further comprise an attR sequence 3′ to the donor polynucleotide sequence.

Described herein are eukaryotic genomes comprising a donor polynucleotide sequence; and an attL sequence 3′ to the donor polynucleotide sequence, wherein the attL sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC. In some embodiments, the eukaryotic genomes further comprise an attR sequence 3′ to the donor polynucleotide sequence.

Described herein are eukaryotic genomes comprising a donor polynucleotide sequence; and an attL sequence 5′ or 3′ to the donor polynucleotide sequence, wherein the attL sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC; and an attR sequence 5′ or 3′ to the donor polynucleotide sequence, wherein the attR sequence comprises a sequence selected from the group consisting of: SEQ ID NOs: 17-18, 7145, 7147, 7150, GCATCCCC, TATTCGAT, GGGCAACC, GGGCACCC, CAAGTTC, ACCGCC, CATATGT, 7219, 7224, 7231, 7232, 7237, 7242, ATGGTGGGC, 7252, GCCATTTC, TCAGCTCCA, 7269, 7270, 7275, 7276, 7281, GGGTC, TTCATGAG, ATGGTGGGC, 7301, 7306, 7311, 7316, GGGATCCC, 7326, GCCGA, 7336, 7341, 7346, 7351, 7356, 7361, 7366, 7371, 7376, 7381, 7386, 7391, AGGCGG, 7401, and GGATGC.

Exemplary conserved cores of recombination sites are shown in Table 2.

TABLE 2
Recombination Site Sequences

| SEQ | Serine | Nucleotide |
|---|---|---|
| ID NO: | Recombinase | Sequence |
| 17 | MG178-4 core | AAACATCGCATC |
| 18 | MG178-9 core | GAACTGGCACAT |
| | MG178-10 core | GCATCCCC |
| | MG178-11 core | TATTCGAT |

In some embodiments, the attL sequence and the attR sequence are the same.

In some embodiments, the attL sequence is a recombined sequence of a first attachment site sequence and a second attachment site sequence. In some embodiments, the attR sequence is a recombined sequence of a first attachment site sequence and a second attachment site sequence.

### Donor Polynucleotides

Serine recombinases described herein can provide for integration of polynucleotides (e.g., donor polynucleotides) of large sizes. In some embodiments, the donor polynucleotide comprises a size of at least about 1 kilobase (kb), 2 kb, 3 kb, 4 kb, 5 kb, 6 kb, 7 kb, 8 kb, 9 kb, 10 kb, 20 kb, 30 kb, 40 kb, 50 kb, or more than 50 kb. In some embodiments, the donor polynucleotide comprises a size of at least about 15 kb, 20 kb, 25 kb, 30 kb, 35 kb, 50 kb, 100 kb, 200 kb, 300 kb, 400 kb, or 500 kb. In some embodiments, the donor polynucleotide comprises a size of about 200 base pairs (bp) to about 500 kb, 200 bp to about 250 kb, or 200 bp to about 100 kb. In some embodiments, the donor polynucleotide comprises a size of about 1 kb to about 10 kb, about 1 to about 7.5 kb, about 1 to about 5 kb, about 1 to about 3 kb, about 2 to about 10 kb, about 2 to about 7.5 kb, about 2 to about 5 kb, about 2 to about 3 kb, about 3 to about 10 kb, about 3 to about 7.5 kb, or about 3 to about 5 kb. In some embodiments, the donor polynucleotide comprises a size of about 10 kb to about 500 kb, 10 kb to about 400 kb, 10 kb to about 300 kb, 10 kb to about 200 kb, 10 kb to about 100 kb, about 10 kb to about 75 kb, about 10 kb to about 50 kb, about 10 kb to about 30 kb, about 20 kb to about 100 kb, about 20 to about 75 kb, about 20 kb to about 50 kb, about 20 kb to about 30 kb, about 30 kb to about 100 kb, about 30 kb to about 75 kb, or about 30 kb to about 50 kb. In some embodiments, the donor polynucleotide comprises a size of about 10 to about 500, 20 to about 400, 10 to about 300, 10 to about 200, or 10 to about 100. In some embodiments, the donor polynucleotide is circular. In some embodiments, the donor polynucleotide is linear.

In some embodiments, the donor polynucleotide encodes a therapeutic, a reporter, or a marker.

In some embodiments, the reporter comprises a fluorescent protein. In some embodiments, the fluorescent protein is GFP, EBFP, EBFP2, Azurite, mKalamal, ECFP, Cerulean, CyPet, YFP, Citrine, Venus, YPet, RFP, CFP, or a derivative thereof.

In some embodiments, the reporter is acetohydroxyacid synthase (AHAS), alkaline phosphatase (AP), beta galactosidase (LacZ), beta glucuronidase (GUS), chloramphenicol acetyltransferase (CAT), horseradish peroxidase (HRP), luciferase (Luc), nopaline synthase (NOS), octopine synthase (OCS), luciferase, or a derivative thereof.

In some embodiments, the marker is an antibiotic resistance marker. In some embodiments, the antibiotic resistance marker is kanamycin, spectinomycin, streptomycin, ampicillin, carbenicillin, bleomycin, erythromycin, polymyxin B, tetracycline, chloramphenicol, neomycin, zeocin, or a derivative thereof.

In some embodiments, the marker is a cell surface marker. In some embodiments, the cell surface marker is a membrane protein, a sugar moiety, or a small molecule (for example biotin) presented on the cell surface. In some embodiments, the cell surface marker is a CD3, B2M, CD4, CD8, CD28, HLA proteins, MHC complex, streptavidin, or avidin. In some embodiments, the cell surface marker is an antibody for example an IgG, or an antibody fragment for example an scFv, or an Fc. In some embodiments, the cell surface marker can be bound by a specific antibody. In some embodiments, the cell is analyzed for expression of the cell surface marker by flow cytometry.

### Delivery and Vectors

Disclosed herein, in some embodiments, are nucleic acid sequences encoding a serine recombinase or a serine recombinase gene editing system disclosed herein.

In some embodiments, the nucleic acid encoding the serine recombinase or the serine recombinase gene editing system is a DNA, for example a linear DNA, a plasmid DNA, or a minicircle DNA. In some embodiments, the nucleic acid is an RNA, for example a mRNA.

Described herein are vectors comprising: a) a nucleic acid encoding a serine recombinase comprising at least about 80% sequence identity to any one of SEQ ID NOs: 21-7060 and 7105-7142; and b) one or more regulatory elements. Further described herein are vectors comprising a nucleic acid encoding a serine recombinase comprising at least about 80% sequence identity to any one of SEQ ID NOs: 21-7060 and 7105-7142, wherein the vector is selected from the group consisting of: a plasmid, a nanoplasmid, a phagemid, a phage derivative, a bacmid, a bacterial artificial chromosome (BAC), a minicircle, a doggybone, a yeast artificial chromosome (YAC), and a cosmid.

In some embodiments, the nucleic acid encoding the serine recombinase or the serine recombinase gene editing system is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of: pSF-CMV-NEO-NH2-PPT-3XFLAG, pSF-CMV-NEO-COOH-3XFLAG, pSF-CMV-PURO-NH2-GST-TEV, pSF-OXB20-COOH)-TEV-FLAG (R)-6His, pCEP4 pDEST27, pSF-CMV-Ub-KrYFP, pSF-CMV-FMDV-daGFP, pEFla-mCherry-N1 vector, pEF1a-tdTomato vector, pSF-CMV-FMDV-Hygro, pSF-CMV-PGK-Puro, pMCP-tag (m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the one or more regulatory elements comprises a promoter, an enhancer, an intron, a microRNA, a linker, a splicing element, or a polyA signal. In some embodiments, the promoter is selected from a constitutive promoter, an inducible promoter, a mini promoter, or a derivative thereof. In some embodiments, the promoter is selected from the group consisting of: CMV, CBA, EF1a, CAG, PGK, TRE, U6, UAS, T7, Sp6, lac, araBad, trp, Ptac, p5, p19, p40, Synapsin, CaMKII, GRK1, polH, EM7, OpIE1, and a derivative thereof. In some embodiments the promoter is a U6 promoter. In some embodiments, the promoter is a CAG promoter.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-21YF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the serine recombinase or a serine recombinase gene editing system is delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the serine recombinase or the serine recombinase gene editing system is introduced into the cell in any suitable way, either stably or transiently. In some embodiments, the serine recombinase or the serine recombinase gene editing system is transfected into the cell. In some embodiments, the cell is transduced or transfected with a nucleic acid construct that encodes the serine recombinase or the serine recombinase gene editing system. For example, a cell is transduced (e.g., with a virus encoding the serine recombinase or the serine recombinase gene editing system), or transfected (e.g., with a plasmid encoding the serine recombinase or the serine recombinase gene editing system) with a nucleic acid that encodes the serine recombinase or the serine recombinase gene editing system, or the translated the serine recombinase or the serine recombinase gene editing system. In some embodiments, the transduction is a stable or transient transduction. In some embodiments, a plasmid expressing the serine recombinase or the serine recombinase gene editing system is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

In some embodiments, delivery of the serine recombinase or the serine recombinase gene editing system to the target nucleic acid site comprises delivering a nucleic acid comprising an open reading frame encoding the serine recombinase or the serine recombinase gene editing system. In some embodiments, the nucleic acid comprises a promoter. In some embodiments, the open reading frame encoding the serine recombinase or the serine recombinase gene editing system is operably linked to the promoter. In some embodiments, the promoter is a ribonucleic acid (RNA) pol III promoter.

In some embodiments, delivery of the serine recombinase or the serine recombinase gene editing system to the target nucleic acid site comprises delivering a capped mRNA containing the open reading frame encoding the serine recombinase or the serine recombinase gene editing system. In some embodiments, delivery of the serine recombinase or the serine recombinase gene editing system to the target nucleic acid site comprises delivering a translated polypeptide. In some embodiments, delivery of the serine recombinase or the serine recombinase gene editing system to the target nucleic acid site comprises delivering a deoxyribonucleic acid (DNA) encoding the serine recombinase or the serine recombinase gene editing system operably linked to a ribonucleic acid (RNA) pol III promoter.

### Lipid Nanoparticles

Disclosed herein, in certain embodiments, are lipid nanoparticles comprising the serine recombinase or the serine recombinase gene editing system of the disclosure for delivery into a cell.

In some embodiments, the lipid nanoparticle comprises the serine recombinase or the serine recombinase gene editing system or a nucleic acid encoding the serine recombinase or the serine recombinase gene editing system. In some embodiments, the lipid nanoparticle comprises the one or more components of the serine recombinase gene editing system. In some embodiments, the lipid nanoparticle comprises the serine recombinase or a nucleic acid encoding the serine recombinase. In some embodiments, the lipid nanoparticle comprises the donor polynucleotide.

In some embodiments, the lipid nanoparticle is tethered to the serine recombinase gene editing system.

Lipid nanoparticles as described herein can be 4-component lipid nanoparticles. Such nanoparticles can be configured for delivery of RNA or other nucleic acids (e.g., synthetic RNA, mRNA, or in vitro-synthesized mRNA) and can be generally formulated as described in WO2012135805A2. Such nanoparticles can generally comprise: (a) a cationic lipid, (b) a neutral lipid (e.g., DSPC or DOPE), (c) a sterol (e.g., cholesterol or a cholesterol analog), or (d) a PEG-modified lipid (e.g., PEG-DMG).

The cationic lipid referred to herein as “C12-200” is disclosed by Love et al., Proc Natl Acad Sci USA. 2010 107:1864-1869 and Liu and Huang, Molecular Therapy. 2010 669-670. Cationic lipid formulations can include particles comprising either 3 or 4 or more components in addition to polynucleotide, primary construct, or RNA (e.g., mRNA). As an example, formulations with certain cationic lipids, include, but are not limited to, 98N12-5 and may contain 42% lipidoid, 48% cholesterol and 10% PEG (C14 or greater alkyl chain length). As another example, formulations with certain lipidoids include, but are not limited to, C12-200 and may contain 50% cationic lipid, 10% disteroylphosphatidyl choline, 38.5% cholesterol, and 1.5% PEG-DMG.

In some embodiments, the cationic lipid nanoparticle comprises a cationic lipid, a PEG-modified lipid, a sterol, and a non-cationic lipid. In some embodiments, the cationic lipid nanoparticle has a molar ratio of about 20-60% cationic lipid: about 5-25% non-cationic lipid: about 25-55% sterol; and about 0.5-15% PEG-modified lipid. In some embodiments, the cationic lipid nanoparticle comprises a molar ratio of about 50% cationic lipid, about 1.5% PEG-modified lipid, about 38.5% cholesterol, and about 10% non-cationic lipid. In some embodiments, the cationic lipid nanoparticle comprises a molar ratio of about 55% cationic lipid, about 2.5% PEG-modified lipid, about 32.5% cholesterol, and about 10% non-cationic lipid. In some embodiments, the cationic lipid is an ionizable cationic lipid, the non-cationic lipid is a neutral lipid, and the sterol is a cholesterol. In some embodiments, the cationic lipid nanoparticle has a molar ratio of 50:38.5:10:1.5 of cationic lipid:cholesterol: PEG2000-DMG:DSPC or DMG:DOPE. In some embodiments, lipid nanoparticles as described herein can comprise cholesterol, 1,2-dioleoyl-sn-glycero-3-phosphoethanolamine (DOPE), 1,1′-((2-(4-(2-((2-(bis(2-hydroxydodecyl)amino)ethyl) (2-hydroxydodecyl)amino)ethyl) piperazin-1-yl)ethyl) azanediyl)bis(dodecan-2-ol) (C12-200), and DMG-PEG-2000 at molar ratios of 47.5:16:35:1.5.

### Methods for Gene Editing

Described herein, in some embodiments, are methods for gene editing, comprising: a) providing or identifying a first attachment site sequence in a host genome; b) providing a nucleic acid comprising a donor polynucleotide and a second attachment site sequence to a host cell; and c) contacting the host cell with a serine recombinase comprising at least about 80% sequence identity to any one of SEQ ID NOs: 21-7060 and 7105-7142 or a nucleic acid encoding the serine recombinase, wherein the first attachment site sequence and the second attachment site sequence are capable of recombination.

In some embodiments, the first attachment site sequence is endogenous in the host genome.

In some embodiments, the first attachment site sequence is provided using viral delivery. In some embodiments, viral delivery comprises use of a virus, wherein the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the first attachment site sequence is provided using a transposase. In some embodiments, the transposase is transposase (Tnp) Tn5, Sleeping Beauty transposase, or a Tn7 transposon. In some embodiments, the gene editing system comprises an enzyme with transposase activity. Additional enzymes with transposase activity include, but are not limited to, retrons and IS200/IS605 transposons.

In some embodiments, the first attachment site sequence is provided using a nuclease. In some embodiments, the nuclease is a double-strand nuclease.

In some embodiments, the nuclease is a Type II CRISPR endonuclease. In some embodiments, the nuclease is Cas9. Type II CRISPR systems are considered the simplest in terms of components. In Type II CRISPR systems, the processing of the CRISPR array into mature crRNAs does not require the presence of a special endonuclease subunit, but rather a small trans-encoded crRNA (tracrRNA) with a region complementary to the array repeat sequence; the tracrRNA interacts with both its corresponding effector nuclease (e.g., Cas9) and the repeat sequence to form a precursor dsRNA structure, which is cleaved by endogenous RNAse III to generate a mature effector enzyme loaded with both tracrRNA and crRNA. Type II nucleases are known as DNA nucleases. Type II nucleases generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand. Exemplary CRISPR Cas9 proteins include, but are not limited to, Cas9 from Streptococcus pyogenes (UniProtKB-Q99ZW2 (CAS9 STRP1)), Streptococcus thermophilus (UniProtKB-G3ECR1 (CAS9 STRTR)), Staphylococcus aureus (UniProtKB-J7RUA5 (CAS9 STAAU), Campylobacter jejuni (UniProtKB-QOP897 (CAS9 CAMJE)), Campylobacter lari (UniProtKB-A0A0A8HTA3 (A0A0A8HTA3 CAMLA), Helicobacter canadensis (UniProtKB-C5ZYI3 (C5ZYI3 9HELI)), and Francisella tularensis subsp. Novicida (UniProtKB-A0Q5Y3 (CAS9_FRATN). Additional Type II nucleases are described in International Patent Application Publication WO 2021/226363, WO 2022/159758, and WO 2022/056324.

In some embodiments, the nuclease is a CRISPR nuclease. In some embodiments, the CRISPR nuclease is a Class 2 Type II SpCas9 or a Class 2 Type V-A Cas12a (previously Cpf1). In some embodiments, the Type V-A nuclease has a guide RNA of 42-44 nucleotides compared with approximately 100 nt for SpCas9. In some embodiments, the Type V-A nuclease results in staggered cut sites. In some embodiments, the Type V-A nuclease results in staggered cut sites to facilitate directed repair pathways, such as microhomology-dependent targeted integration (MITI).

In some embodiments, the nuclease is a Type V CRISPR endonuclease. Type V CRISPR systems are characterized by a nuclease effector (e.g., Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, Type V systems are capable of using the effector nuclease itself to cleave pre-crRNAs. Like Type II CRISPR systems, Type V CRISPR systems are known as DNA nucleases. Unlike Type II CRISPR systems, some Type V enzymes (e.g., Cas12a) appear to have a robust single-stranded nonspecific deoxyribonuclease activity that is activated by the first crRNA-directed cleavage of a double-stranded target sequence.

The most commonly used Type V-A enzymes require a 5′ protospacer adjacent motif (PAM) next to the chosen target site: 5′-TTTV-3′ for Lachnospiraceae bacterium ND2006 LbCas12a and Acidaminococcus sp. AsCas12a; and 5′-TTV-3′ for Francisella novicida FnCas12a. In some embodiments the PAM sequence is YTV, YYN, or TTN. Additional Type II nucleases are described in International Patent Application Publication WO 2021/226363.

In some embodiments, the first attachment site sequence is provided using a reverse transcriptase. Reverse transcription is the translation of an RNA template into a complementary DNA. Reverse transcription is performed by enzymes termed reverse transcriptases (RT) that are enzymes with RNA-dependent DNA polymerase activity that create the complementary DNA (cDNA) strand from an RNA template. Some of the RT enzymes also have DNA-dependent DNA polymerase activity to create a double-stranded dsDNA. Reverse transcriptases can be of viral origin (for example HIV, hepatitis B, Moloney murine leukemia virus (MMLV), or avian myeloblastosis virus (AMV)) or bacterial origin (for example group II introns, retrons/retron-like RTs, diversity-generating retroelements (DGRs), Abi-like RTs, CRISPR-associated RTs, and group II-like RTs (G2L)). Reverse transcriptases of eukaryotic origin comprise the telomerase reverse transcriptase that maintains the telomeres of eukaryotic chromosomes. Reverse transcription allows the introduction of site-directed insertions, deletions, and mutations into the cDNA by encoding them in the RNA template.

In some embodiments, the reverse transcriptase is a viral, prokaryotic, or eukaryotic reverse transcriptase. In some embodiments, the reverse transcriptase is an MG151, MG153, or MG160 family reverse transcriptase. In some embodiments, the reverse transcriptase is an MG140, MG146, MG148, MG149, MG151, MG153, MG154, MG155, MG156, MG157, MG158, MG159, MG160, MG163, MG164, MG165, MG166, MG167, MG168, MG169, MG170, or MG176 family reverse transcriptase. In some embodiments, the reverse transcriptase comprises a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to any one of the MG140, MG146, MG148, MG149, MG151, MG153, MG154, MG155, MG156, MG157, MG158, MG159, MG160, MG163, MG164, MG165, MG166, MG167, MG168, MG169, MG170, MG172, MG173, or MG176 family reverse transcriptases or retrotransposases. In some embodiments, the reverse transcriptase comprises a sequence with at least 80% sequence identity to any one of the MG140, MG146, MG148, MG149, MG151, MG153, MG154, MG155, MG156, MG157, MG158, MG159, MG160, MG163, MG164, MG165, MG166, MG167, MG168, MG169, MG170, MG172, MG173, or MG176 family reverse transcriptases or retrotransposases or variants thereof. In some embodiments, the reverse transcriptase is smaller than 300 amino acids. In some embodiments, the reverse transcriptase is smaller than 250 amino acids.

In some embodiments, the methods are used to introduce a modification in the genome of a cell. In some embodiments, the modification is an insertion, deletion, or mutation. In some embodiments, the methods are used to introduce site-directed insertions, deletions, and/or mutations in the genome of a cell (for example an insertion and a mutation). In some embodiments, the methods are used in combination with a nucleic acid template to facilitate site-directed insertions into the genome of a cell. In some embodiments, the cell is a human cell. In some embodiments, the cell genome or a vector comprised in the cell is modified. In some embodiments, the cell genome is modified ex vivo. In some embodiments, the cell genome is modified in vivo.

In some embodiments, the methods described herein further comprise detecting the genome modifications. In some embodiments, after the cell genome is modified, the cell is cultured for a certain amount of time. In some embodiments, the DNA or RNA is extracted and sequenced, and modified sequence areas are mapped and compared with an unmodified sequence. In some embodiments, cells are stained with antibodies for protein products that are translated from the modified nucleic acid, and the resulting stained proteins or polypeptides in the cell are analyzed, for example by flow cytometry.

### Cells

Described herein, in certain embodiments, is a cell comprising the serine recombinase or the serine recombinase system described herein. In some embodiments, the cell (e.g., mammalian cell) comprises the eukaryotic genome described herein. In some embodiments, the cell is a human cell.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NSO), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, WI38, HeLa, Saos, C2C12, L cell, HT1080, HepG2, Huh7, K562, a primary cell, or derivative thereof.

In some embodiments, the cell is a liver cell.

### Kits

In some embodiments, this disclosure provides kits comprising one or more nucleic acid constructs encoding the various components of the serine recombinases described herein, e.g., comprising a nucleotide sequence encoding the components of the serine recombinases capable of modifying a target DNA sequence. In some embodiments, the nucleotide sequence comprises a heterologous promoter that drives expression of the serine recombinases described herein.

In some embodiments, any of the serine recombinases disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In some embodiments, the compositions are constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Bioinformatic Identification of Large Serine Recombinases

This example describes the identification of proteins with large serine recombinase function by a bioinformatic approach.

Putative large serine recombinases (LSRs) were identified in an extensive database of viral, prokaryotic, and eukaryotic proteins. The search resulted in 163,797 non-partial homologs with a score>50. LSRs were further filtered by requiring contigs to have a 1 kbp flank on either side of the LSR, and dereplicated at 90% average amino acid identity (AAI). After dereplication, 8,364 LSRs were globally aligned and a phylogenetic tree was constructed. Closely related contigs lacking the LSRs were identified by searching for contigs containing the two genes flanking approximate proviral boundaries. To ensure the contigs were from a closely related strain, local alignments were performed requiring the two genes to share ≥99% AAI. Precise proviral boundaries were identified by locally aligning the contigs containing and lacking the LSRs at the nucleotide level. Once integration boundaries were delineated, the attL and attR sites flanking the prophage, as well as the attachment sites' common core, were identified by searching for imperfect repeats near the boundaries.

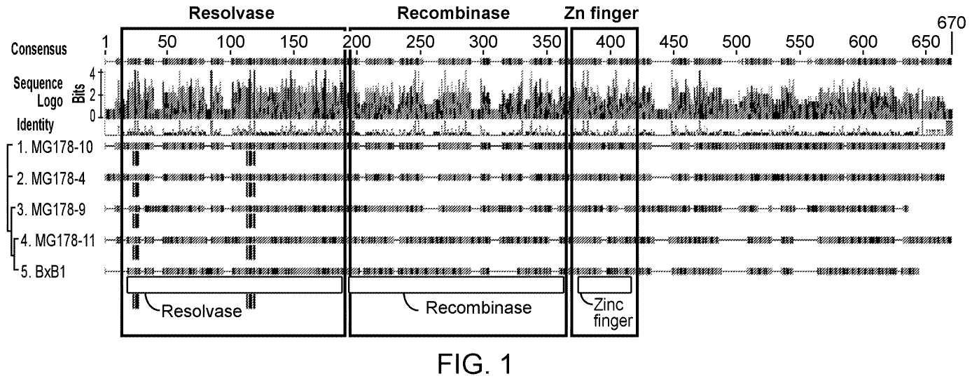

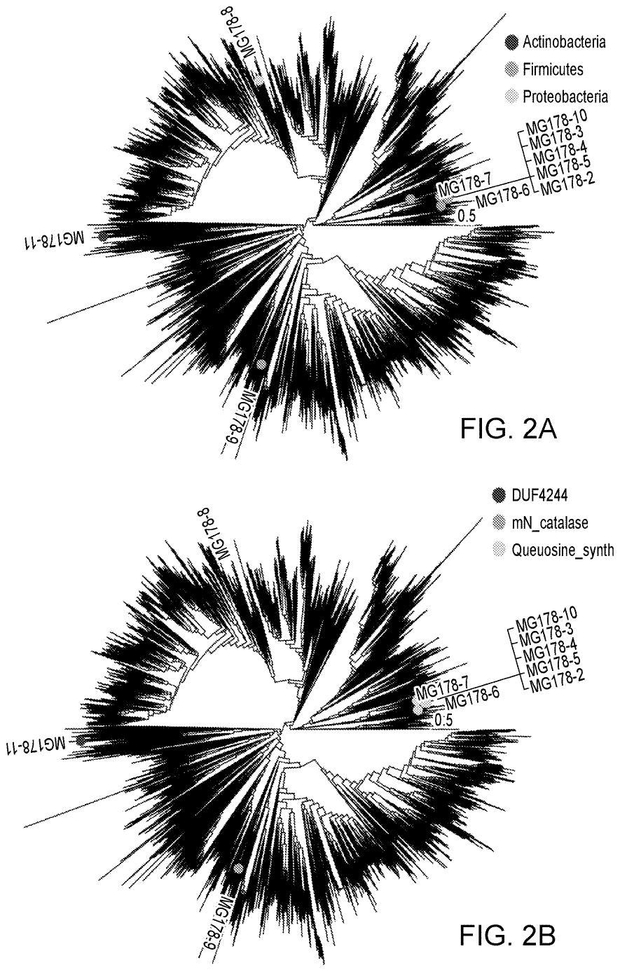

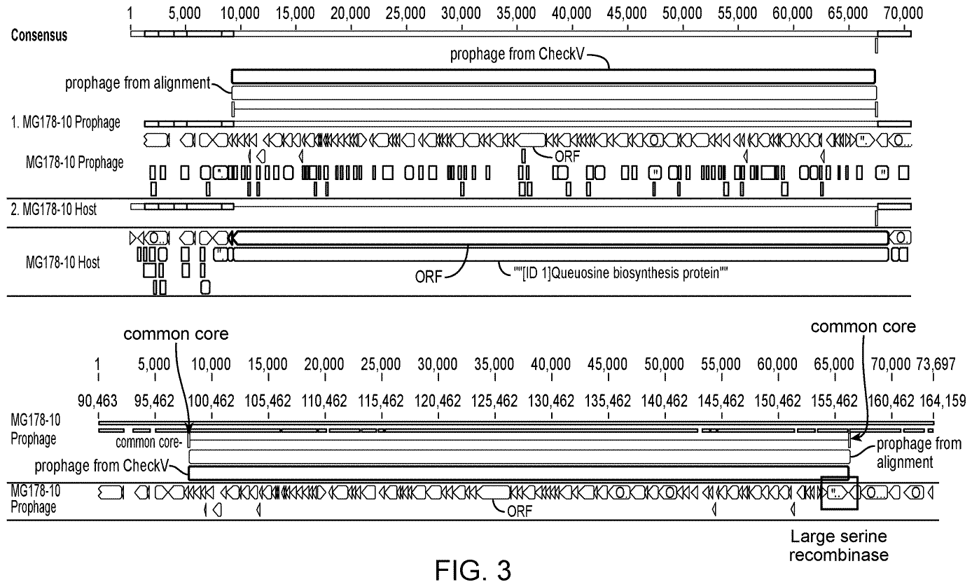

LSR candidates were identified based on the presence of resolvase, recombinase, and Zn-finger domains, as well as catalytic residues required for activity (FIG. 1). Selected LSR candidates belonging to the MG178 family share 26.8% AAI amongst them and <37% AAI with a known Bxb1 LSR reference (FIG. 1). Phylogenetic analysis of LSR candidates indicated that these enzymes are encoded in highly diverse genomes, and prophage boundaries were predicted for many (FIGS. 2A and 2B). The LSR-integrated prophages appeared to be inserted into genes containing Mn_catalase, Queuosine_synth, and DUF4244 Pfam domains (FIG. 2A) and were shown to infect hosts belonging to several Phyla, including Actinobacteria, Firmicutes, and Proteobacteria (FIG. 2B). Prophage genomes mobilized by LSR reached nearly 94 kb in length. Prophage boundaries were identified by aligning the contigs containing the LSR with highly similar contig sequences lacking the LSRs, which likely represent the host without the integration event (FIG. 3). With integration boundaries delineated, the attachment site's common cores were identified by searching for repeats near the boundaries (FIG. 3).

### Example 2. In Vitro Assay of Serine Recombinase Activity

### In Vitro Recombination Reactions

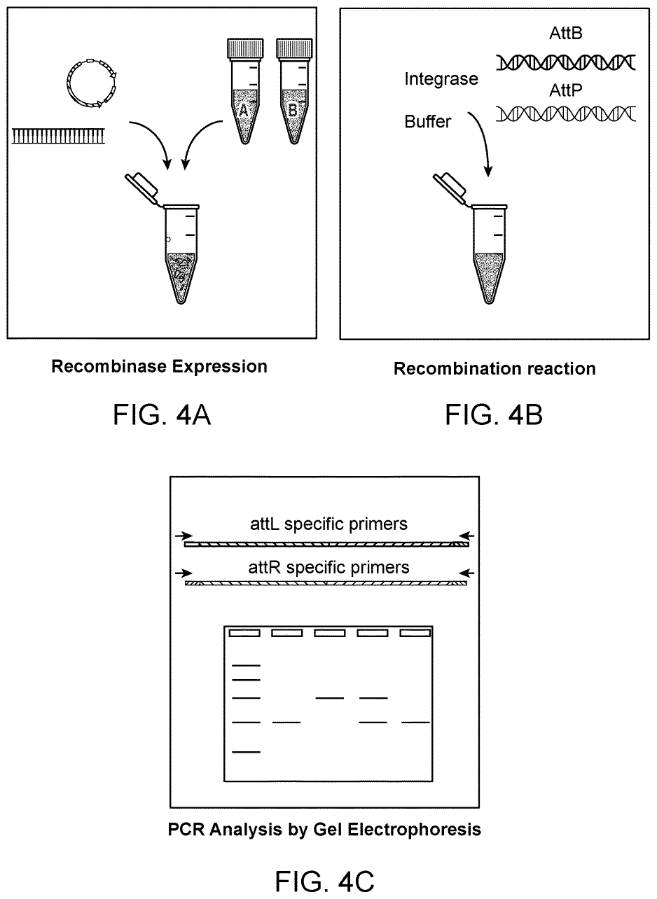

To test the functionality of the serine recombinases, the attP and attB sites from the attL, attR, and common core sequences from the native integrated prophage genomic context (SEQ ID NOs: 1-18, GCATCCCC and TATTCGAT) were determined bioinformatically and tested in in vitro recombination reactions. The attB and attP sites were synthesized in gene fragments˜300 bp in length with primer-binding sites unique to each attachment site end (FIG. 4C). Serine recombinases were expressed in vitro, while negative controls included in vitro expression reactions without template (null) (FIG. 4A). Negative recombination reaction controls were set up in 10 μL reactions using 50 ng of attB, 50 ng of attP, recombination buffer (20 mM HEPES pH 7.5, 50 g/mL bovine serum albumin (BSA), 2 mM TCEP, 5 mM MgCl₂, 100 mM KCl, 5 mM spermidine, 2 mM ZnCl2, and 5% glycerol) and 1 μL of null reaction (no recombinase template). Experimental conditions included 50 ng of attB, 50 ng of attP, and 1 μL in vitro-expressed recombinase (FIG. 4B). Recombination reactions were incubated at 30° C. for 1 hour and diluted with water at 1:10. PCR reactions were then performed with attL-(attB5 and attP3) or attR-(attB3 and attP5) specific primer sets (FIG. 4C) and analyzed on a 2% agarose gel to determine amplification and size of resulting products. Product forming reactions were Sanger sequenced and aligned to the predicted attL and attR sequences determined bioinformatically.

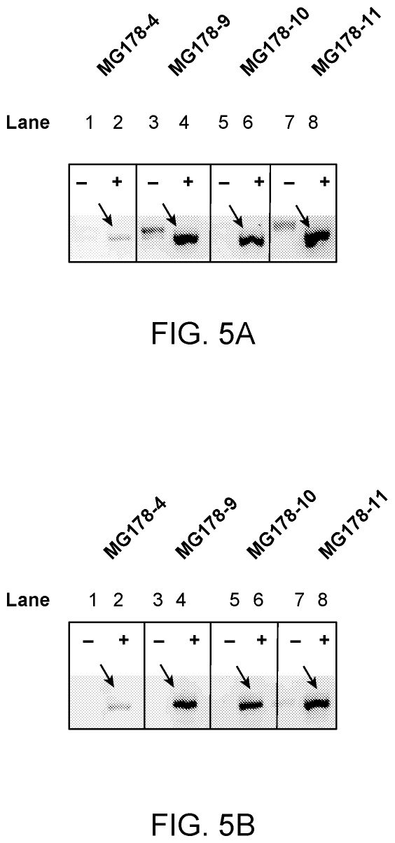

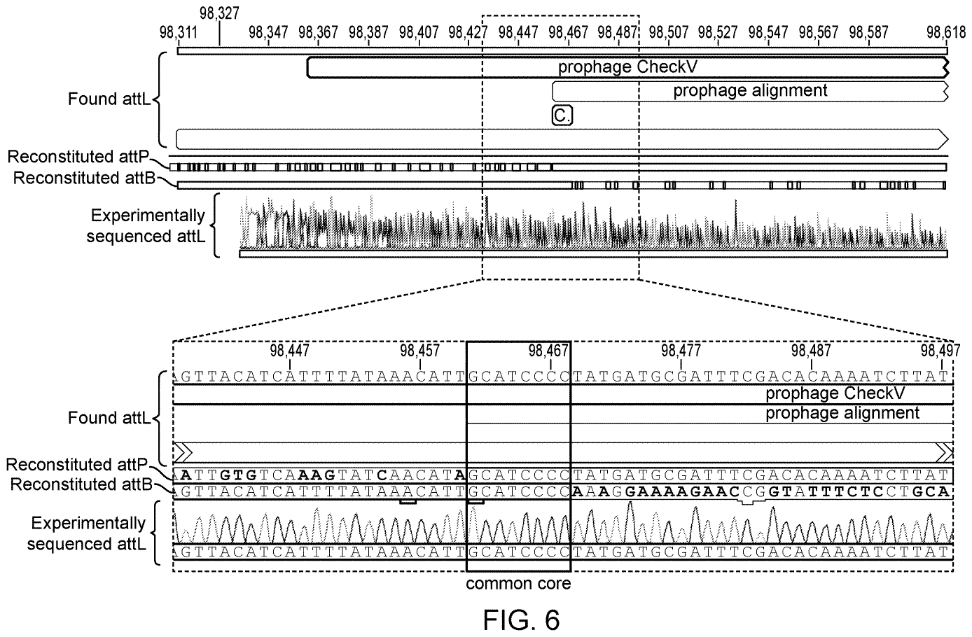

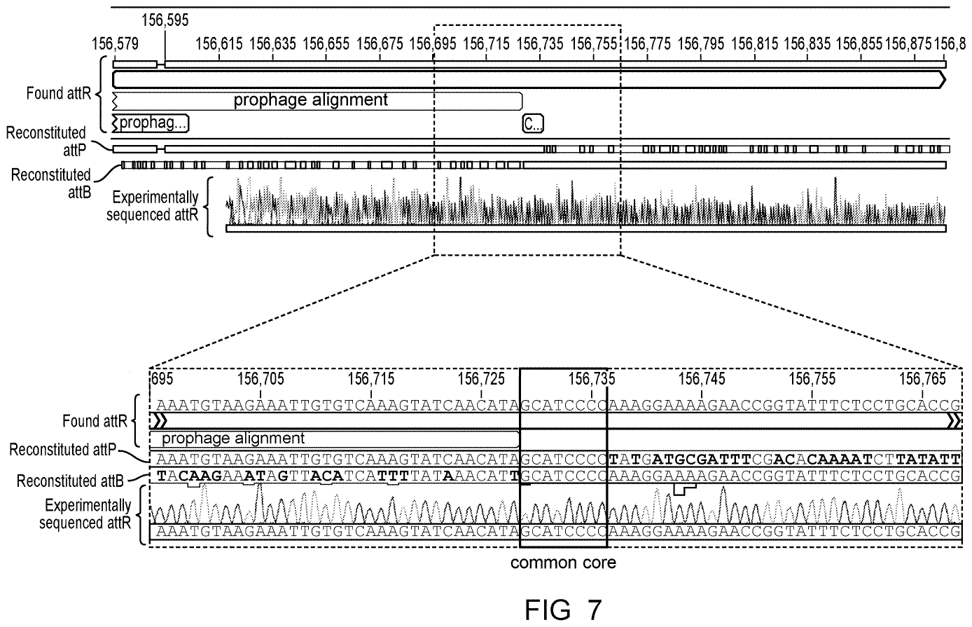

The LSR candidates were expressed in vitro and added to a reaction buffer with putative attB and attP dsDNA fragments (FIGS. 4A-4C). Four LSRs (MG178-4 (SEQ ID NO: 21), MG178-9 (SEQ ID NO: 22), MG178-10 (SEQ ID NO: 23), and MG178-11 (SEQ ID NO: 24) were active based on formation of both recombination products of attL and attR (FIG. 5). PCR amplifications were then Sanger sequenced to confirm crossover events of the predicted attB- and attP-forming attL and attR sequences. The results show that Sanger sequencing confirmed the recombination events of the active recombinase-containing reactions for both predicted attL and attR and conservation of the common core in both reactants and recombination products (FIG. 6 and FIG. 7).

### Example 3. Prophetic—In Cell Plasmid Recombination

Recombinases are tested for their activity in human cells by synthesizing the attP fragment into a donor plasmid (pDonor) with the attP site upstream of a promoterless mCherry coding ORF. attB fragments are synthesized into a pTarget plasmid encoding a pCMV promoter upstream of the attB site without a downstream coding ORF. When co-transfected with the active recombinase, the pCMV promoter of pTarget is recombined with the pDonor mCherry, and the junction of the pCMV promoter to the mCherry drives transcription and translation of the mCherry coding region. Efficiency of the recombinase is compared to the negative control of a cell population transfected with both pDonor and pTarget without the recombinase plasmid.

### Example 4. Prophetic-Landing Pad Activity in Mammalian Cells

To introduce exogenous donor DNA into the human genome using large serine recombinases, the landing pad, an attP or attB sequence site, is (1) found to be endogenous to the human genome sequence, or (2) introduced using viral delivery or by way of a transposable element, (3) integrated into the genome using HDR coupled with a nuclease, or (4) reverse transcribed into the genome using a targeted reverse transcriptase.

After introduction or identification of the landing pad (either an attP or attB) site to the genome, LSR activity to the genome is determined by using a DNA donor comprising (1) a promoter driven fluorescent protein construct or (2) a promoterless fluorescent coding construct with the cognate attachment (attB/attP) site and/or (3) an antibiotic resistance marker or (4) a screenable cell surface marker. The donor is introduced into the cell as a plasmid, a minicircle, a Bacterial Artificial Chromosome, a nanoplasmid, or a linear dsDNA construct to integrate into the landing pad.

Along with introducing the donor into the cell, the LSR is transfected into the cell using either, (1) a plasmid encoding for the transcription and translation of the LSR, (2) an mRNA coded for LSR translation, or (3) a purified protein. Landing pad efficiency is determined by flow analysis in the case of a fluorescent protein and/or cell surface marker donor, or colony formation under selective conditions and subsequent PCR analysis of exogenous/endogenous DNA junction formation.

### Example 5. In Silico Identification of Large Serine Recombinases in the MG178 Family

In Silico Identification of LSR and their Putative Attachment Sites

Putative large serine recombinases (LSRs) were identified with the following modifications: LSR domain specific (PF00239 and PF07508) hmm searches resulted in 987,835 non-partial homologs with a score>50 and length>450 aa. LSRs with at least a 1 kbp flank on either side were dereplicated at 99% AAI resulting in 146,897 non-redundant homologs. LSR attL and attR sites were identified.

### Results

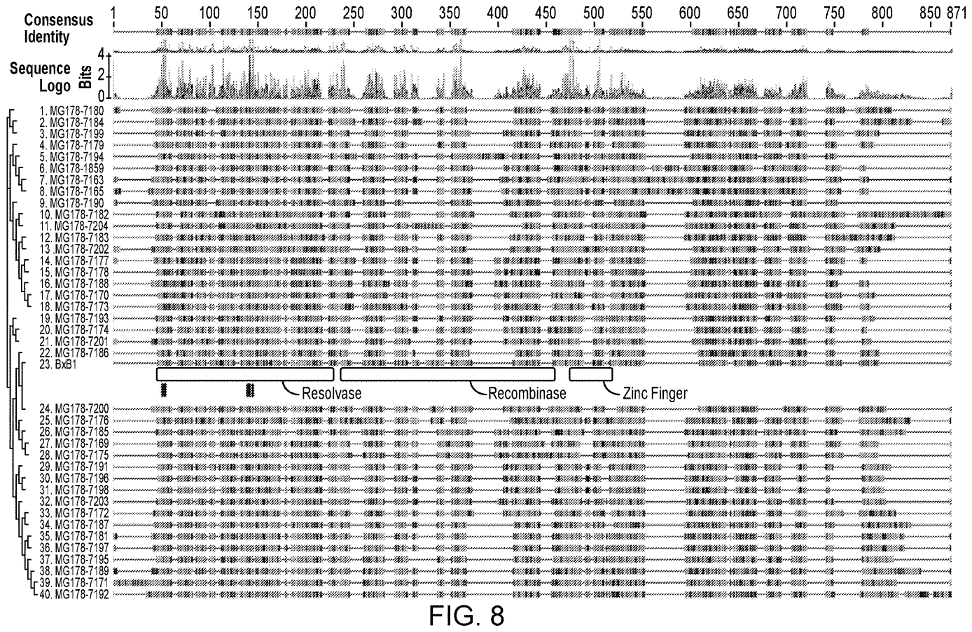

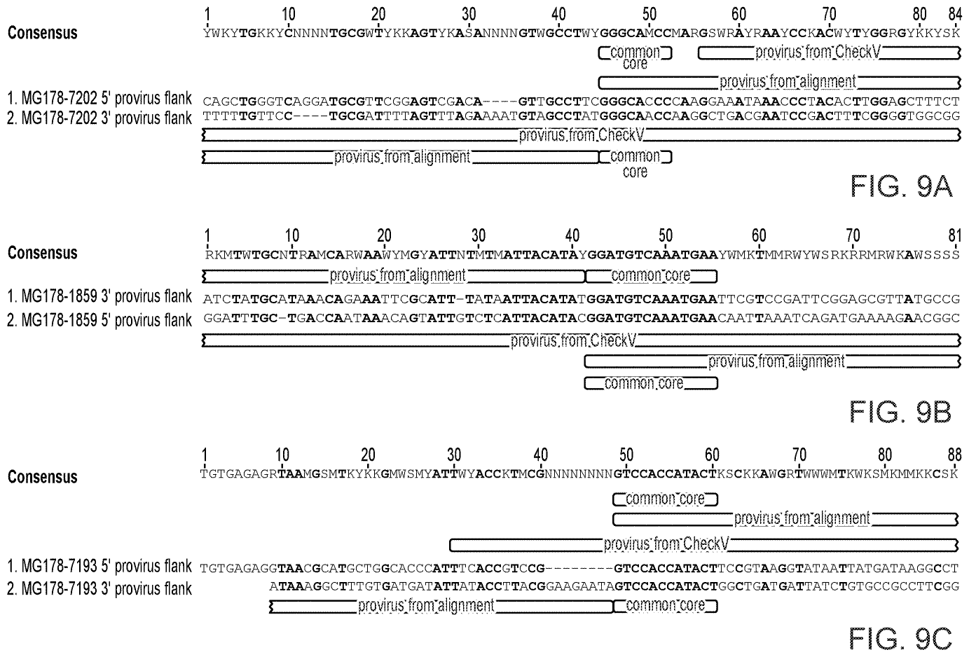

LSR candidates were identified based on the presence of resolvase, recombinase, and Zn-finger domains, as well as catalytic residues required for activity (FIG. 8). Selected LSR candidates belonging to the MG178 family share 16.9% AAI amongst them and <18% AAI with a known BxB1 LSR reference. The LSRs identified in this work integrate into genes belonging to the radical SAM superfamily, glycosyl hydrolases family 18, helix-turn-helix domain of transposase family ISL3, peptidase family M3, transcriptional regulators, outer membrane protein beta-barrel domain, type II/IV secretion system protein, acetyltransferase (GNAT) family, MFS_1 like family, magnesium chelatase, and manganese containing catalase Pfam domains, as well as into unannotated genes, transfer-messenger RNAs, T-box leader RNAs, and intergenic regions. The viruses that encode the identified LSRs infect a diverse array of hosts including Actinobacteria, Proteobacteria, Bacteroidetes, Firmicutes, Lentisphaerota, Fusobacteria, Candidatus Aminicenantes, and unknown phyla. Proviral genomes mobilized by the LSRs reached nearly 62 kbp in length. Proviral boundaries were identified by aligning the contigs containing the LSR with highly similar sequences lacking the LSRs, which likely represent the host without the integration event. With integration boundaries delineated, the LSR's attachment site's common cores were identified by searching for direct repeats near the boundaries. Perfect and imperfect repeats representing the common cores were identified by finding conserved regions in local alignments of the proviral boundaries (FIGS. 9A and 9B), and in cases where alignments showed no conservation, repeats were visually identified, and the alignments were manually refined (FIG. 9C).

### Example 6. In Cell Plasmid Recombination

### In Cell Plasmid Recombination Reactions

150,000 HEK293T cells, seeded for 24 hours, were transfected with 1 μg of integrase, 0.5 μg of attP containing plasmid, and 0.5 μg of attB containing plasmid using LT1 transfection reagent. Transfected cells were incubated for 48 hours at 37° C. and then harvested using 0.25% Trypsin reagent, washed in 1×PBS and stained with Fixable near-IR Live/Dead reagent. Processed cells were then analyzed by flow cytometry using a negative control to gate for cells not expressing eGFP (integrase) nor mCherry (recombination) and eGFP only (integrase expression only). Cell analysis was performed by calculating the percentage of cells positive for mCherry (recombination) over the total number of cells positive for eGFP (integrase transfection and expression).

### Results

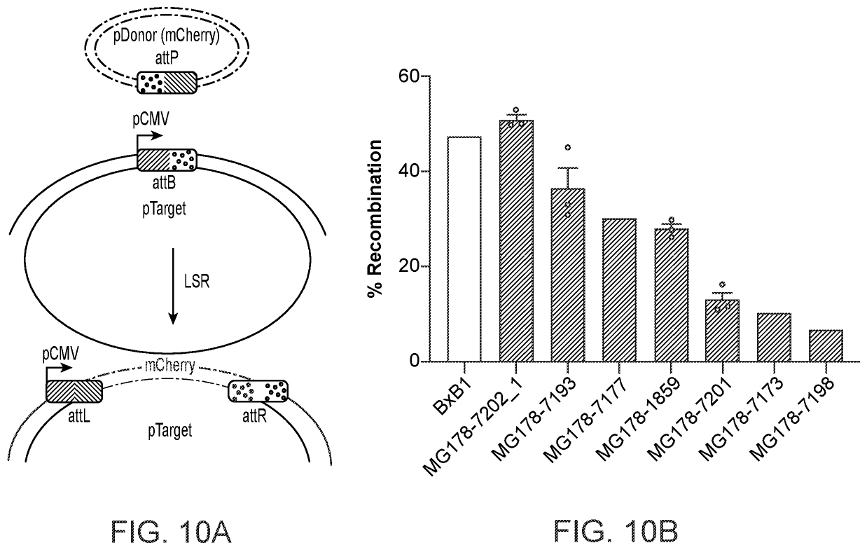

Selected LSRs recombinases were tested for their activity in human cells by synthesizing the recombinase, as well as the attP fragment into a donor plasmid (pDonor) with the attP site upstream of a promoterless mCherry coding ORF. The attB fragments were synthesized into a pTarget plasmid encoding a pCMV promoter upstream of the attB site without a downstream coding ORF (FIG. 10A). When co-transfected with the active recombinase, the pCMV promoter of pTarget will be recombined with the pDonor mCherry, and the junction of the pCMV promoter to the mCherry will drive transcription and translation of the mCherry coding region. Efficiency of the recombinase was compared to the negative control of a cell population transfected with both recombinase plasmid and pDonor without the pTarget plasmid. Of the recombinases tested, MG178-7202 (SEQ ID NO: 7140) was active only to a single predicted attachment site 1 (GGGCACCC) at 50% of all transfected cells, MG178-7193 (SEQ ID NO: 7131) was active at up to 45%, MG178-1859 (SEQ ID NO: 1848) and MG178-7177 (SEQ ID NO: 7115) were active up to 30%, MG178-7201 (SEQ ID NO: 7139) recombined at up to 20%, and MG178-7173 (SEQ ID NO: 7111) and MG178-7198 (SEQ ID NO: 7136) recombined at less than 20% of total transfected cells (FIG. 10B).

### Example 7. In Vitro Recombination of LSR Systems

### In Vitro Testing of Recombination

To test the functionality of the serine recombinases, the attP and attB sites were predicted from the attL, attR and common core sequences from the native integrated prophage genomic context. attB and attP sites were synthesized in gene fragments of approximately 300 bp in length with primer binding sites unique to each attachment site end (FIG. 4C). Serine recombinases were expressed in vitro, while negative controls included in vitro expression reactions without template (null) (FIGS. 4A-4C). Negative recombination reaction controls were set up in 10 μL reactions using 100 ng of attB, 100 ng of attP, recombination buffer (20 mM HEPES pH 7.5, 50 μg/ml bovine serum albumin (BSA), 2 mM TCEP, 5 mM MgCl2, 100 mM KCl, 5 mM spermidine, 0.2 mM ZnCl, and 5% glycerol) and 1 μL of spent null reaction (no recombinase template). Experimental conditions included 100 ng of attB, 100 ng of attP and 1 μL in vitro expressed recombinase. Recombination reactions were incubated at 30° C. for 1 hour and diluted with water at 1:10. PCR reactions were then performed with recombinase specific primer sets (SEQ ID NOs: 7407-7411) and run on a 2% agarose gel to determine amplification and size of resulting products.

### Results

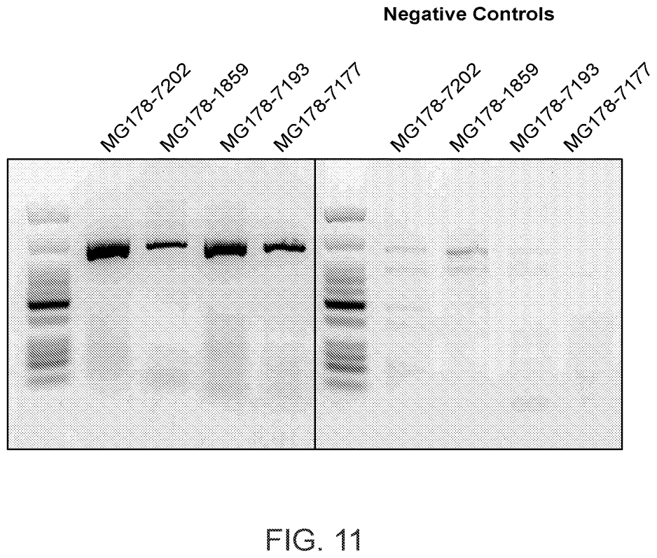

LSR candidates were expressed in vitro and added to a reaction buffer with in cell recombination determined attB and attP dsDNA fragments. Four LSR (MG178-7202, SEQ ID NO: 7096; MG178-7193, SEQ ID NO: 7087; MG178-1859, SEQ ID NO: 1848; and MG178-7177, SEQ ID NO: 7071) were active based on strong PCR amplified recombination products that were not observed in negative control conditions containing no recombinase enzyme (FIG. 11).

### Example 8. In Cell Plasmid Recombination by Active MG178 Candidates

### In Cell Plasmid Recombination Reactions

24 hour seeded 150,000 HEK293T cells were transfected with 1 μg of integrase, 0.5 μg of attP containing plasmid and 0.5 μg of attB containing plasmid using LT1 transfection reagent. Transfected cells were incubated for 48 hours at 37° C. and then harvested using 0.25% Trypsin reagent, washed in 1×PBS and stained with Fixable near-IR Live/Dead reagent. Processed cells were then analyzed by flow cytometry using a negative control to gate for cells not expressing eGFP (integrase) nor mCherry (recombination) and eGFP only (integrase expression only). Cell analysis was performed by calculating the percentage of cells positive for mCherry (recombination) over the total number of cells positive for eGFP (integrase transfection and expression).

### Results

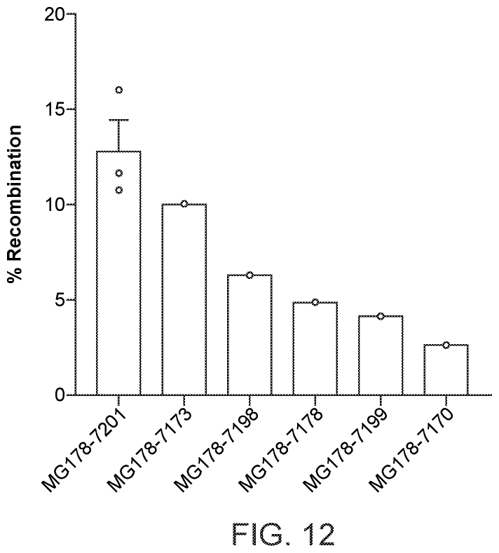

Selected LSRs recombinases were tested for their activity in human cells by synthesizing the recombinase, as well as the attP fragment into a donor plasmid (pDonor) with the attP site upstream of a promoterless mCherry coding ORF. The attB fragments were synthesized into a pTarget plasmid encoding a pCMV promoter upstream of the attB site without a downstream coding ORF (FIGS. 10A and 10B). When co-transfected with the active recombinase, the pCMV promoter of pTarget will be recombined with the pDonor mCherry, and the junction of the pCMV promoter to the mCherry will drive transcription and translation of the mCherry coding region. Efficiency of the recombinase was compared to the negative control of a cell population transfected with both recombinase plasmid and pDonor without the pTarget plasmid. Of the recombinases tested, MG178-7178 (SEQ ID NO: 7072), MG178-7199 (SEQ ID NO: 7093), MG178-7170 (SEQ ID NO: 7064) recombined at less than 5% of total transfected cells, while MG178-7201 (SEQ ID NO: 7095) promoted recombination above 15% (FIG. 12).

### Example 9. Human Cell Recombination as a Result of Plasmid Dosage

Active levels of recombinase activity in cells are easily affected by the amount of recombinase, target and donor plasmids introduced into the cell. To test the most effective ratio of plasmid dosing, we altered the recombinase plasmid individually, or with the target and donor plasmids.

### In Cell Plasmid Recombination Reactions

24 hour seeded 150,000 HEK293T cells were transfected with varying levels (0.1-1 μg) of integrase, 0.1-0.5 μg of attP containing plasmid and 0.1-0.5 μg of attB containing plasmid using LT1 transfection reagent. Transfected cells were incubated for 48 hours at 37° C. and then harvested using 0.25% Trypsin reagent, washed in 1×PBS and stained with Fixable near-IR Live/Dead reagent. Processed cells were then analyzed by flow cytometry using a negative control to gate for cells not expressing eGFP (integrase) nor mCherry (recombination) and eGFP only (integrase expression only). Cell analysis was performed by calculating the percentage of cells positive for mCherry (recombination) over the total number of cells positive for eGFP (integrase transfection and expression).

### Results

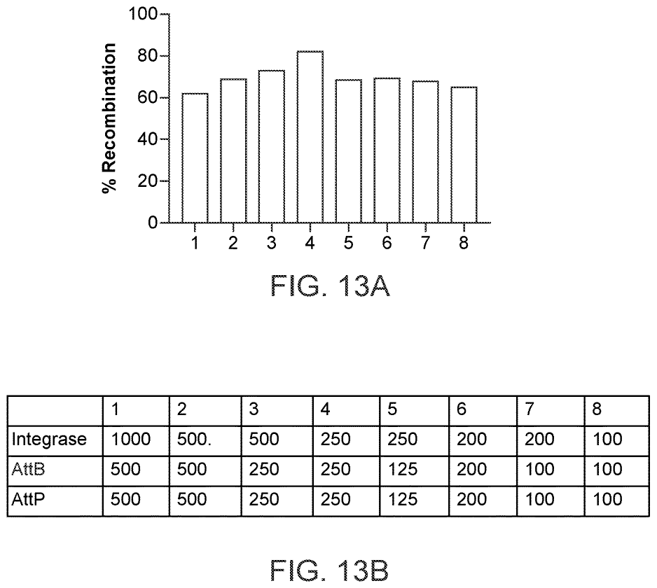

Candidate MG178-7202 (SEQ ID NO: 7096) LSR recombinase was tested for their activity in human cells by dosing varying levels of integrase, donor, and target plasmids for measured increases in recombination efficiency. MG178-7202 was found to be the most active at integrase plasmid amounts equal to target and donor plasmids, at 250 ng per transfection. This represents a 30% increase in recombinase activity given by the concentration of plasmids in cells (FIG. 13A and FIG. 13B).

### Example 10. In Cell Attachment Site Minimization

### Construction of Minimized Attachment Sites

Attachment site minimization is crucial to understanding the limits of recombinase activity into the eukaryotic cell. A smaller attachment site footprint allows for the streamlined incorporation of the attB or attP site to any locus of interest in the human genome by means of a dsDNA donor or a RNA templated addition to the genome for attachment site incorporation. In order to identify a minimized sequence, a series of AttP and attB variant sites were synthesized with the previously described promoterless mCherry for attP and a markerless promoter with attB. Decreasing sizes of both attB and attP were benchmarked against the 300 nt active attachment site (SEQ ID NO: 7100). For MG178-7202, attB sequences tested correspond to 108, 88, 68, 58, 48, 46, 44, 42, 40 38, 36, 32, 28 nt in size (SEQ ID NOs: 7188-7200), and attP sequences were tested at 108, 88, 68, 58, 48 nt (SEQ ID NOs: 7183-7187). For MG178-7193, attB sites were tested at 112, 92, 72, 62, and 52 nt (SEQ ID NOs: 7206-7210) and attP sites were tested at sizes 112, 92, 72, 62 and 52 nt (SEQ ID NOs: 7201-7205).

### In Cell Plasmid Synthesis and Recombination Reactions

24 hour seeded 150,000 HEK293T cells were transfected 250 ng of integrase, 250 ng of attP containing plasmid and 250 ng of attB containing plasmid using LT1 transfection reagent. Transfected cells were incubated for 48 hours at 37° C. and then harvested using 0.25% Trypsin reagent, washed in 1×PBS and stained with Fixable near-IR Live/Dead reagent. Processed cells were then analyzed by flow cytometry using a negative control to gate for cells not expressing eGFP (integrase) nor mCherry (recombination) and eGFP only (integrase expression only). Cell analysis was performed by calculating the percentage of cells positive for mCherry (recombination) over the total number of cells positive for eGFP (integrase transfection and expression).

### Results

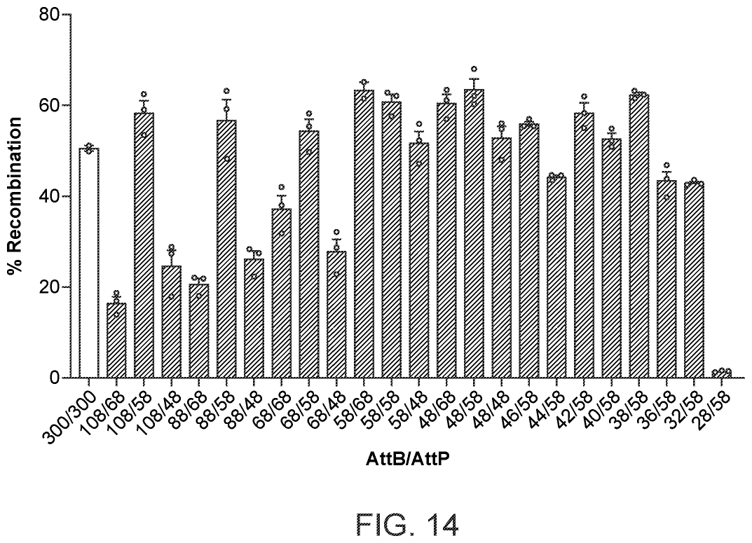

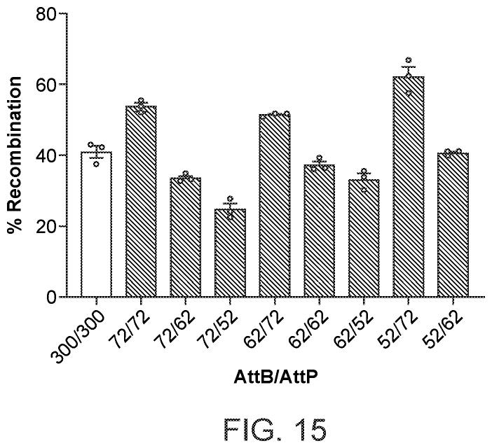

A range of minimal attachment sites were tested for two LSR recombinases. All the combinations were benchmarked to the original 300 bp attachment sites for comparison of recombination efficiency. Highest efficiency recombination occurred between 58 nt for attP to 48 nt attB site for MG178-7202 (FIG. 14). Recombinase activity was further detected with a 48 nt attP and down to 32 nt for attB. MG178-7193 attachment sites were shown to be most active at 52/72 attB/attP sizes, but recombinase activity was measured down to 52/62 attB/attP (FIG. 15).

### Example 11. MG178s Purification and In Vitro Activity Assay

Isolating pure and functional proteins is essential for extensive in vitro analysis of biochemical properties and mechanistic studies. MG178 candidates were expressed and purified to obtain proteins of sufficient quantity and quality for such characterizations. MG178-1859 (SEQ ID NO: 1848) was expressed as an N-terminal Sumo-fusion protein in a Carbenicillin-resistant pMGF expression vector, while MG178-7202 (SEQ ID NO: 7096) was expressed as N-terminal Sumo-fusion protein in a Kanamycin-resistant pET28 expression vector. All constructs were expressed in E. coli.

### Protein Expression

Protein expression plasmids were transformed into competent cells and cultured overnight in 50 mL 2×YT media (1.6% tryptone, 1% yeast extract, 0.5% NaCl) with 100 μg/mL Carbenicillin or 50 μg/mL Kanamycin at 37° C. depending on the expression vector. The next day, 7 mL from each overnight culture was used to inoculate 1000 mL TB media (1.2% Tryptone, 2.4% Yeast Extract, 0.4% Glycerol, 17 mM Potassium Phosphate Monobasic, 72 mM Potassium Phosphate Dibasic) containing 100 μg/L Carbenicillin or 50 g/mL Kanamycin at 37° C., and cultures were grown, shaking at 37° C. At OD600≈0.8-1.2, cultures were cooled on ice before induction with 0.3 mM IPTG and 0.2% w/v L-(+)-Arabinose and further incubation at 16° C., shaking, for approximately 18 hrs. Cultures were then harvested by centrifugation at 6,000×g for 10 min, and pellets were resuspended in Nickel_A Buffer (50 mM HEPES, 500 mM NaCl, 10 mM MgCl2, 1 mM EDTA, 20 mM imidazole, 5% glycerol, pH 7.5)+protease inhibitors (EDTA-free)+2 mg/mL lysozyme (Lysozyme from Chicken Egg White, Research Product International L38100) and stored at −80° C. Culture samples were taken pre- and post-induction, and cells were pelleted via centrifugation (15,000×g, 1.5 min) and resuspended in 100 μL 2× Laemmli Buffer per 1 OD cells.

### Protein Purification

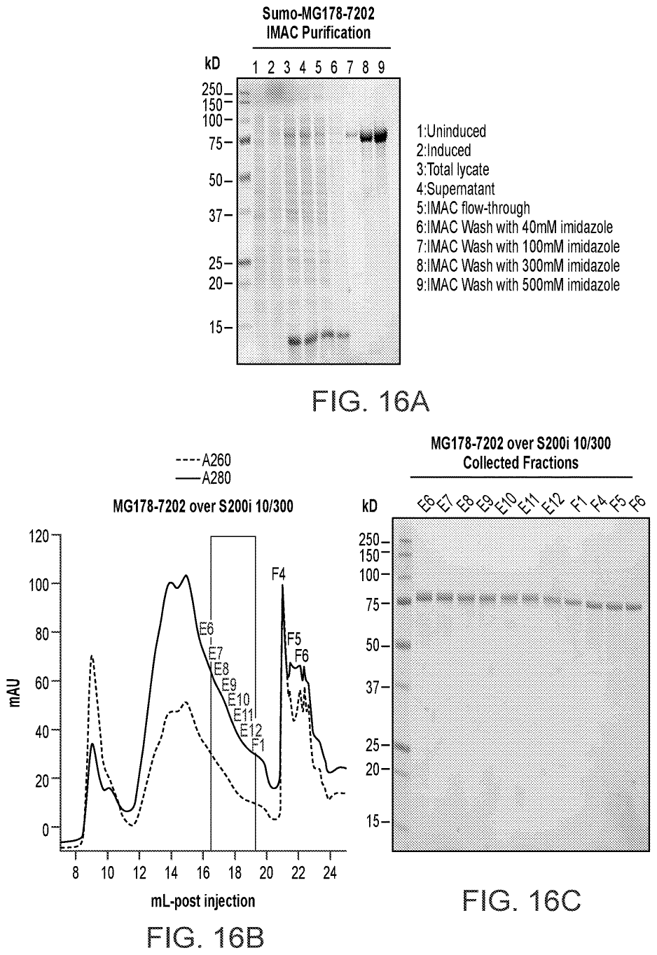

MG178-7202 (SEQ ID NO: 7096) is shown here as an example of the protein purification process. Expressed proteins have the following sequence architecture: 6×His-(GS) 1-Sumo-GSGSGGSGS-PSP-SV40 NLS-HA-MG178. Cell pellets were thawed and the volume supplemented to 120 mL with Nickel_A buffer with 0.5% β-octylglucoside (P1P1P1, CI-00234). Samples were sonicated in an ice-water bath at 75% amplitude for a total processing time of 3 min using a 5 s on/15 s off cycle. Lysates were clarified by centrifugation at 30,000×g for 15 min, and supernatants batch bound to 5 mL Ni-NTA resin (for ≥15 min. Samples were loaded onto a gravity column and washed with 10 CV Nickel_A Buffer and washed again with 10 CV Nickel_A2 Buffer (Nickel_A Buffer+100 mM imidazole), then eluted in 2 CV Nickel_B Buffer (Nickel_A Buffer+300 mM imidazole) and 2 CV Nickel_B2 Buffer (Nickel_A Buffer+500 mM imidazole). Fractions collected with Nickel_B and Nickel_B2 Buffer were pooled before concentrating in a 50 kDa MWCO concentrator. Samples were taken throughout the purification process and run on an SDS-PAGE protein gel, which was imaged on a ChemiDoc in the stain-free channel following 5 min UV activation. These gels were used to track the progress of purification throughout the protocol (FIG. 16A). MG178-7202 sample was then filtered through a 0.22 μm cellulose acetate membrane before loading onto an S200i 10/300 GL column and run into SEC buffer (50 mM HEPES, 250 mM NaCl, 10 mM MgCl2, 1 mM EDTA, 5% glycerol, 0.5 mM TCEP, pH to 7.5) to further isolate purified protein (FIGS. 16B and 16C).

### In Vitro Testing of Recombination

To test the functionality of the serine recombinases, we predicted the attP and attB sites from the attL, attR and common core sequences from the native integrated prophage genomic context. attB and attP sites are synthesized in gene fragments ˜300 bp in length with primer binding sites unique to each attachment site end. Serine recombinases were expressed in vitro, while negative controls included in vitro expression reactions without template (null). Negative recombination reaction controls were set up in 10 μL reactions using 100 ng of attB, 100 ng of attP, recombination buffer (20 mM HEPES pH 7.5, 50 μg/ml bovine serum albumin (BSA), 2 mM TCEP, 5 mM MgCl₂, 100 mM KCl, 5 mM spermidine, 0.2 mM ZnCl, and 5% glycerol) and 1 μL of spent null reaction (no recombinase template). Experimental conditions included 100 ng of attB, 100 ng of attP and 1 μL in vitro expressed recombinase. Recombination reactions were incubated at 30° C. for 1 hour and diluted with water at 1:10. PCR reactions were then performed with specific primer sets (SEQ ID NOs: 7416 and 7417) and run on a 2% agarose gel to determine amplification and size of resulting products. Product forming reactions were Sanger sequenced and aligned to predicted attL and attR sequences determined bioinformatically.

### Results

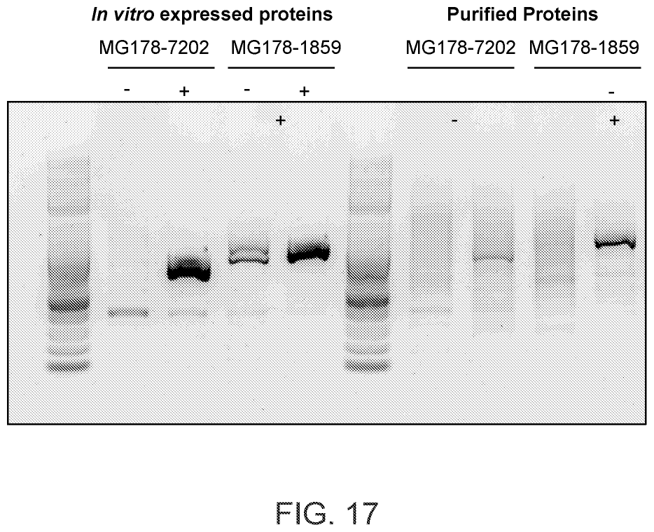

LSR candidates were expressed in vitro and added to a reaction buffer with in cell recombination determined attB and attP dsDNA fragments. Two LSR candidates (MG178-7202 (SEQ ID NO: 7096) and MG178-1859 (SEQ ID NO: 1848) were active based on strong PCR amplified recombination products that are not observed in negative control conditions containing no recombinase enzyme, and more specific when compared to the in vitro expressed control (FIG. 17). Results support prior observations of active protein expression from cell-free extracts for in vitro recombination activity (Example 7).

### REFERENCES

Anzalone A V, Gao X D, Podracky C J, Nelson A T, Koblan L W, Raguram A, Levy J M, Mercer J A M, Liu D R. Programmable deletion, replacement, integration and inversion of large DNA sequences with twin prime editing. Nat Biotechnol. 2022, 40 (5): 731-740. doi: 10.1038/s41587-021-01133-w. Epub 2021 Dec. 9. PMID: 34887556; PMCID: PMC9117393.
Durrant M G, Fanton A, Tycko J, Hinks M, Chandrasekaran S S, Perry N T, Schaepe J, Du P P, Lotfy P, Bassik M C, Bintu L, Bhatt A S, Hsu P D. Systematic discovery of recombinases for efficient integration of large DNA sequences into the human genome. Nat Biotechnol. 2022 Oct. 10. doi: 10.1038/s41587-022-01494-w. PMID: 36217031
Smith M C M. Phage-encoded Serine Integrases and Other Large Serine Recombinases. Microbiol Spectr. 2015 August;3 (4). doi: 10.1128/microbiolspec.MDNA3-0059-2014. PMID: 26350324
Robert C. Edgar, Search and clustering orders of magnitude faster than BLAST, Bioinformatics, Volume 26, Issue 19, 1 Oct. 2010, Pages 2460-2461, doi.org/10.1093/bioinformatics/btq461
Nayfach, S., Camargo, A. P., Schulz, F. et al. CheckV assesses the quality and completeness of metagenome-assembled viral genomes. Nat Biotechnol 39, 578-585 (2021). doi.org/10.1038/s41587-020-00774-7
Price M N, Dehal P S, Arkin A P (2010) FastTree 2-Approximately Maximum-Likelihood Trees for Large Alignments. PLOS ONE 5 (3): e9490. doi.org/10.1371/journal.pone.0009490
Katoh K, Standley D M. MAFFT multiple sequence alignment software version 7: improvements in performance and usability. Mol Biol Evol. 2013; 30 (4): 772-780. doi: 10.1093/molbev/mst010
Steinegger, M., Söding, J. MMseqs2 enables sensitive protein sequence searching for the analysis of massive data sets. Nat Biotechnol 35, 1026-1028 (2017). doi.org/10.1038/nbt.3988

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

SEQUENCE LISTING

| | SEQ | | | |
|---|---|---|---|---|
| | ID | | | |
| Category | NO: | Description | Type | Sequence |
| MG178 | 7105 | MG178- | protein | MSKLSNKLNSIRVAIYVRVSTHYQVDKDSLPLQREELV |
| recombinases | | 7163 large | | AYAKYVLNAKSHEVFEDAGYSAKNTDRPDYQQMMA |
| | | serine | | RVRTGEFSHILVWKLDRISRNLLDFATMYDELKKLGVT |
| | | recombinase | | FVSKNEQFDTSSAMGEAMLKIILVFAELERKMTAERVT |
| | | | | AVMVSRAGTGQWNGGRIPYGYDYDKESETFSINPDEE |
| | | | | KIVLKIFELYEERQSILYVARTLNDSGIRHRSGKEWSPT |
| | | | | TVSIILKNIFYTGAYRYNYFDMSKRGSRQDIKPESEWVI |
| | | | | IPEHHPAIITEERWRNVLLILEQNRRGWKASGKTYHRK |
| | | | | NVHVFAGLLTCQLCGATMSATSSSRELKGGYRPSIYAC |
| | | | | MSHRKGTGCTNKYISDTVLGPFVLNYIANLIKAKASFG |
| | | | | KTTSPETLHKKILRGDMFSEVTGIKEHGLMELYSLYRS |
| | | | | QISGIKYIPASIDTEETDSASERDILLSERRRIERALSRLK |
| | | | | TLYLYAEADMAEKDYIIEKKQLDEQLDKVNSRLEEISK |
| | | | | GLTAQFSISDDELLAKASYFIMSQKLSDKRFVNYRRLL |
| | | | | EETDPRILKDFINTVTCNFCIENGKIRSITFKNGIIHEFMY |
| | | | | KDD |
| MG178 | 7106 | MG178- | protein | MVKKLSKRASEILETINSLRVAIYIRVSTHWQIDKDSLP |
| recombinases | | 7165 large | | LQRSDLINYCKVILGTENYVVFEDAGYSAKNFERPDFQ |
| | | serine | | KMMARVRSGEFSHILVWKIDRISRNLLDFATMYQELK |
| | | recombinase | | ELGVTFVSKNEQFDTSTAIGEAMLKIILVFAELERNMTS |
| | | | | ERVTATMLARASDGKWNGGKVPFGYSYDKEEKSFVIN |
| | | | | EAESRVVRLMYDLYDEQHSLLAVSKELNRRGYRSRKG |
| | | | | AEWSPVTVGNIMRSPFYIGSLRYNYRRESGPSFTFRPES |
| | | | | EWVMIEDHHPQTVTHEQWNRVVSTMRSQRRGQPGHG |
| | | | | KSFNRGNVHIFAGIITCGYCGSLMRASQDRPRKDGWRP |
| | | | | SIYTCSRKRVSNDCPNKYVSDISVGPFVLNFIANMIRAR |
| | | | | KSFGVSTSIETLQKKLLRGDAFKDVIGIGQSGLKELYRH |
| | | | | FKGTQDQEQNFMADPGSTATTEQEEREVLLSEKRRKE |
| | | | | RAINRLKALYLYSEDEDAISETDYIVERKRLIDSLETIDA |
| | | | | RLAEIDKALSEQATLSDEEFIAKASYFIMAQQLSDKRYI |
| | | | | DYERFVRKVDPQIVKNFVNLVCSNFCIKNGRVVSIRFK |
| | | | | NGIELQFFYNDL |
| MG178 | 7107 | MG178- | protein | MRTAAYARYSSDNQREASLDDQLRNCRAYCVRQGWP |
| recombinases | | 7169 large | | APTVYQDAEISGSRTDRADYQRLLRDARQFDVILVDDL |
| | | serine | | TRFGRDKEELGTIIKRLRFHGVRLIGVSDGIDTARKGYK |
| | | recombinase | | VETGLRGLMSELFLDDLADKTHRGLTGRALAGASAGG |
| | | | | LPFGYIVTETGQRAIDETKAAIVRRIYADYLAGRSPREI |
| | | | | VSALNAERVPAPRGGQWHLSGVYGDVRRGLGILANPI |
| | | | | YTGRQVWNRSHWIKHPDTGRRVRQERPPSEWITTEQP |
| | | | | DLAIIDAATWTAAQARIRAASPKRTGDKQGGPGRPAR |
| | | | | YLLSGILHCDQCDGPMVIVDRYRYGCSRHKDQGDAVC |
| | | | | TSRTRVPRALVEDALVAGVRAELLSDAAFRRYQRATA |
| | | | | EALKRAAPDTAAAKRSLAAAEQVQQNIMAALRAGIIT |
| | | | | PSTRTELIAAEAAVTTATSELQALQSIQPTHILPRARER |
| | | | | WNNVVARLTDTRDIPAARDALRELIGNRVTVKNENGE |
| | | | | LFAEIAASECQIKLVAGAGFEPATFGL |
| MG178 | 7108 | MG178- | protein | MAYCIYLRKSRMDLEAEARGEGDTLARHEKILLELAK |
| recombinases | | 7170 large | | KLKLNIGAIYREIVSGETIAARPVVQRLLSEVGQGMWE |
| | | serine | | GTLVMEIERLARGETIDQGIVAQAYKYSNTKIITPQKIY |
| | | recombinase | | DPQNDFDEEYFEFSLFMSRREYKTINRRLQTGRIASVKE |
| | | | | GKYLGSVPPYGYTIKRLEKQKGNTLAIVPEQAEVVRNI |
| | | | | FHWYTRSENRLGVSLICRRLNEMKIPPAKGDVWVPASI |
| | | | | QTILRNPTYAGKVRWKVRPHRKKMVDGQVVKERPRA |
| | | | | NPSEWIVVDGLHKAIIDWETFELAQQYLSINSSRPCPKF |
| | | | | APIKNPLGGLIICGLCGRKMVRRPHGTRYPDTLMCPNV |
| | | | | ACQNISTQLSIVEERLLEAIDNWMQNYKLQWKDDDFE |
| | | | | SSKTADYRILLKSIKRLDDEIATLDKQMNSIHDLLEQGV |
| | | | | YTTEVFLDRSKKISDRINELKQAKKELNEQYQAFEQQE |
| | | | | VNKKFIIPKIEKVLDIYRTVDDPEIRNQLLHEILEKVIYT |
| | | | | KTQRGNRGGHADNFNLVLYPKLNVSKDFNY |
| MG178 | 7109 | MG178- | protein | MYLTLSEYINSIEKLNAAGTKDTKIRDLLEYYQRYNCS |
| recombinases | | 7171 large | | VTPGPGVFYAVIYARYSSHSQRDESIEGQVREDLEYAS |
| | | serine | | RNNMIVLGVYIDRALTGKEIDKRISFQQMIKDSTRGKW |
| | | recombinase | | QYVITWKVDRFARNRYDAAIYKARLKKHNVRVVYAR |
| | | | | EQIPDGPEGILLESVLEGQAEYYSASLSQNIRRGQEDNA |
| | | | | MECKVNGSIPLGYRVGADRRFEIDPGTAPIIHTIFELYD |
| | | | | AGHTYQDIEGYLNSRGYKTQKGGPFNKNSFNRILKND |
| | | | | RYIGTYRYKEVTVENGMPAIIDKELFESVQRKIEKNRK |
| | | | | ARAHKKSDMNFLLTTKLYCGLCGKAMIGESGTGKLG |
| | | | | GKYYYYTCVGKKRDKTCKKKPIKKDWIENLVIQETVR |
| | | | | LILQDDMINEIADKVMEYQAREADHTILHSLQIQLNDT |
| | | | | EKAIKNLIAAIEQGIITPSTKNRLEELEDEKIRIMNGIAEE |
| | | | | DTIQPVVERDQILFYLKHFQNGDVSDPQYCQTLIDVFV |
| | | | | NAVYVYDDRIVITYNYSGAHNSVTLEQIEQALGESEGS |
| | | | | DTVSSAPC |
| MG178 | 7110 | MG178- | protein | MSRKLIRVAIYARFSCDKQRDASIEDQLYECNKYAERH |
| recombinases | | 7172 large | | GYTVVMQYCDYAMSGRSDDRPNFLRMIEDAKTGMFE |
| | | serine | | IILVWKMDRFARNIEEQYYYEHVLRKNGGVTFESVKE |
| | | recombinase | | NIAGTSIEATSTKALNALFAEVRSRQSAEDTMRGMLGK |
| | | | | ARKCQYLGYPLYGYSHDGDKITLDPEKAPIAKRIHVDF |
| | | | | LSGVAPKQILDWLLSIGVKTARGKDPGYGFVVSMLKN |
| | | | | VRYAGVYMWGVKKDEAGNDVLDDLGRPVPLVYIED |
| | | | | GMPAIVSMSQKLSCIEKLGFRRRLKTNADYLLSGKLIC |
| | | | | SKCKEPMHGETAIGGSGIEYWRYSCRGKRKACLGSFN |
| | | | | KETVEQGVVSGVREMLRDAALVDYLIDRHIQFRDERQ |
| | | | | SKATIEAVRKDIRAVKKQRDNLLSAVAEGLDFNHVKP |
| | | | | KIAALDAQERALEKRIEELKREQNSVSREELRAFFSDM |
| | | | | SKGALSDEQITLTFVSKVWLYESTAVAVMNFDSCESTQ |
| | | | | YEIELALKKHERPDQQAVRETSKWCPRRDSNARHPL |
| MG178 | 7111 | MG178- | protein | MSYLMYLRKSRADKEAELRGEGETLARHEQILSEFAA |
| recombinases | | 7173 large | | KMDLPIGAIYKEIVSGETISSRPKMQQLLIEVMQGHWD |
| | | serine | | GVLVMEIERLARGDTKDQGTVAEAFKFSNTKIITPIKTY |
| | | recombinase | | DPLNEYDEEYFEFNLFMSRREYKTINRRIQRGRIAAFND |
| | | | | GWYIAGTAPYGYKKVKRKGDKGYTLDIVDNEAQVVR |
| | | | | MIYDLYTHGELQDDGSYLRFGSYQIKDRLNDLHIQSRS |
| | | | | GSTWSAAAIIDILRNPAYAGYQRWSWRKVQKKLVGG |
| | | | | NIVESRPKNDDCSKVKGRFEAIITDEQFELAQKIREGKP |
| | | | | TPIRSTNALQNPLSGLVYCEKCGTLMTRQPSNTKDHYP |
| | | | | VLRCPNSKCTNISAPLYLIEQKLIEGLAEWADEYELNW |
| | | | | PKKDWEDTEISTATYQHSVDSMQGKLDTITKQLSAAY |
| | | | | DLLEQGIYTLQVFQERRSTLEKQKEESETELQRLNRAL |
| | | | | EISQARARAKREFLPTIRHIVDTYWTIDDVLVKNTMLK |
| | | | | EVLEKVDYLKTERNKKGGKGNANFSIYFYPRVPKY |
| MG178 | 7112 | MG178- | protein | MKIAAAYLRVSTERQDEYSLDSQLKLIRDYAASNDYIV |
| recombinases | | 7174 large | | PDEFVFIDDGISGRSAEKRPEFLRMIGTAKEKEPPFEAIL |
| | | serine | | VWKFSRFARNQEESIVYKSMLARCGVTVVSISEPLAEG |
| | | recombinase | | AFGSLIERILEWMDEFYSIRLSGEVRRGMTERVSRGEPV |
| | | | | TIPSFGYDITDKTYVPNRDANTVRRIYADYLAGKGVSTI |
| | | | | ARELALEGVKTRRGKSPENRWVQYILENPVYIGKLRW |
| | | | | SPDGKANYTRSEVGDSAMLVDGTHEHIIDDATWAAVQ |
| | | | | DKIARSKRGRIPYQRREQPVDWMLKGLVRCDTCGSTL |
| | | | | VYTSTACPSMQCHKYAHGACPTSHALSIAKANRAVIA |
| | | | | ALESCAASLSFPVAPQTVKPVADEPDYSKLIKQEQQKL |
| | | | | RRLMDAYEEGVYTIEEFAARRAKLDDKIAQLKKQAEK |
| | | | | NTPAIIDVQEYKVRVLDVLDIIKSDNVSEADKNTALKAI |
| | | | | LSYIVYEKANNRLALYFYF |
| MG178 | 7113 | MG178- | protein | MRTAAYLRYSSDQQRDASIRDQLRNIETYCDRQGWQK |
| recombinases | | 7175 large | | PLVFQDEAVSGARSDRPGYRALIKAARDRQFEVLLVD |
| | | serine | | DLSRLSRDHIEAAQAVRLLKFLGVRLIGVSDGLDTARN |
| | | recombinase | | GYKLETGMRGLMAELYLDDLAEKTHRGLMGQALDG |
| | | | | YSAGGLPYGYASVHDGHGHRRVILEEQAQWVRWMFD |
| | | | | RYIRGHSPRAIAAELNALGIPSARGKTWCLTAIYPDAK |
| | | | | HVGILGNPIYNGRQIWNRTKWIKDPSTGRRKRILRPESE |
| | | | | WVITEHPELKIVDDDTWSAARDRALKTRARTARQREN |
| | | | | LRRASISGGRGPKYLFSGLLRCACCGSSYVVVDRYRYG |
| | | | | CSAHKDRGSAACSNSIKVPRYAIERTLLAGIKEELLSDR |
| | | | | AYRAFESEVRRLLKTAQPDIGEARRAAAKAQAEVDNII |
| | | | | GAIRQGIITPATKQALEEAEGRLDAAKRRIKEIEAWQPT |
| | | | | QMLPRAKQIYRGLAERLERIEDIADAREALRSILGEDIK |
| | | | | LVPENGVLWAELKGGCAALSQITVVAGAGFEPTTFGL |
| MG178 | 7114 | MG178- | protein | MMRAAVYARKSNEQDVQEEVRSVTRQLEHGRAFAES |
| recombinases | | 7176 large | | KGWSVKDDHVFSDDAISGMHGEEKRPGLKLLLATME |
| | | serine | | LVPRPFDVIVMASDDRLMRNQLKVGAVLERIQEAGVD |
| | | recombinase | | LYYYLENRKVDLSTVVGQFMESVHAMAAHDYRVKLA |
| | | | | RHTRDGMKARAKAGFVIGGRTFGYDHVPVDRGEVST |
| | | | | RKTRRVPMTRVINEAQANIVRDIFTRYAAGQSPKQIAR |
| | | | | TLNSRPGILVPGGKHGIPMLRWSRHQVRETLQHVLYT |
| | | | | GVLLSTWNGEQIRVERPDLRIIEDDLWQQTRARFANLR |
| | | | | EQYIRMKHGQLLGRPAHSVESAYLLTGLIQCGVCGRS |
| | | | | MLATTRKRANHTKRKYYQCVSNMNNRRRACDNTLLA |
| | | | | PMDATDAAVLQAVEQSVLNPSILCDAIAHALSKLDGTE |
| | | | | NRQQEQTRLAEELSQLDTQMKHLADSIAKLGGNDTLL |
| | | | | DEVRQREARRQEIGAERSRLESLESLSTLDLGQVEQDL |
| | | | | RRITDEWKGAQGFTNRHPAAARTILKKVLPSKLILTPH |
| | | | | PESRSYSFAGDGAIGPLLGQVVSSQMGKGQGSSGEGR |
| | | | | VRRPNHAATKDSWTTSSACCRSRRRARA |
| MG178 | 7115 | MG178- | protein | MNNTYTLENVIIYLRKSQSDDPAMSVEEVLKKHEDVL |
| recombinases | | 7177 large | | QEFCENEFGKRIPEKQIYREVASGETIEDRPVIQAILKLM |
| | | serine | | ETDSLKGVIVVEPQRFSRGDLQDCGRIVNALRYTNTLA |
| | | recombinase | | ITPQKTYNLNDEYDRKFFEMELTKGSDYLEYYKKIQRR |
| | | | | GREASVKKGNYIGSVTPYGYNKATYMDGNRTCHTLAI |
| | | | | NQIEAEAIRMMADLYLNKGYGFTKIARALDEMGYKPR |
| | | | | KSEKWSPAAIKDIMENPIIIGKIRWNRRKTIKKLCDGEIT |
| | | | | KSRPKAQDYILVDGKHEAILDEETYYKILDKRGKNPRL |
| | | | | RKSKELTNPYAGLLFCGTCGRAMSHKKYKQRKSNTIS |
| | | | | ESMLCNNQANCHTKSVMYSSFEKSVIESLEKAIADFEV |
| | | | | KLQNNNGDITSLRASRIKTLESELKTLLEKDERQKDNL |
| | | | | DDGIYSKEEFLKRNAKVQEQIEATKKALSQVKDSILPEI |
| | | | | DYKEKIVRFQDCLNALTDPNVSAPCKNMLLKSCIDKIV |
| | | | | YHNDSESKAGIGRYVDNPFKLDIFLRL |
| MG178 | 7116 | MG178- | protein | MSEYIMYLRKSRQDDPTETVEEVLKKHEIQLQDFALN |
| recombinases | | 7178 large | | NFHYRIPSDDIYREVVSGETIDDRPMIQAVLKRIEDENI |
| | | serine | | KGVLVIDCQRLTRGDMLDCGIIVHSFRYTNTLIITPQKT |
| | | recombinase | | YTLSDKYDRKFFETELSRGSDYLDYTKEILRRGRDASK |
| | | | | RRGNYIGSVAPYGYDRIKIGKDWTLKPNDESQYVKLIF |
| | | | | ERYTQGVGTFDIVNEIEKLGAKPRNSKSFDYNRITDIIK |
| | | | | NPVYVGKIKVNEDETFKVMQDGKLVKKRKARKEYEL |
| | | | | VDGKHEPIVSQELFDKAQNERKKRTREPNSCVLQNPY |
| | | | | ASLIKCGYCGKAVTLHYGSKKRGGDKYKRLACTGRG |
| | | | | CECVSHNYSEMNDAIIDALRNKLDDIKVQIDTDDNQSN |
| | | | | ANSKIIESLESRLSLNEKKMNDICGYLENGIYTIGIFTKR |
| | | | | KKALEDERTTLEEAIMNAKKESESSSELESKTITLSQAIE |
| | | | | MLKDDSISAKIKNNFLKEIIQVIYYKKDKLGNITLDIYL |
| | | | | R |
| MG178 | 7117 | MG178- | protein | MNPAKIYLLYARVSPKGSTWDCNETSIGVQLADMRTH |
| recombinases | | 7179 large | | ILRQVPDAQFIEVVDEFKSGKNLKRPGVQKILFDLESRP |
| | | serine | | VPWHCLVVWNLDRLSRSLCDAIPIFSKLRDAGCEFISIN |
| | | recombinase | | QEYLSYTGAMARYMLHQTIALAELERGMTSERVSAK |
| | | | | MRWIASEGKIPWGNIPLGYIRKPGVKNTVVIDEPKAEIV |
| | | | | RTIFDMYIAGNLSYTAINKRWPGMIKDRGYLYRILRNP |
| | | | | LYIGELHYAGKVQKAEHPAIIDKEIFEQTQSLLASRRRN |
| | | | | YQRRGIQKYDYLLSGIVRCHCGRQMTGYSVNKKDGK |
| | | | | YFYYKCTSPTCKNAINAETLDSSVLQQIASVFRNKSEIR |
| | | | | ASLQAYLEEQKEKQLAVKIHRNELEKQLAEAKQKQTR |
| | | | | IMDMFLAGVVDQSNAKLWNSELAATRQSVELLEKEIT |
| | | | | ELSVVPEIVFDDIFSDLMKAAEEWTKKIASGEADFATK |
| | | | | RNLIMSVIESLECVKRSDTQIGFKMKLVMSSSCKWWA |
| MG178 | 7118 | MG178- | protein | MAKKYRYGPVAPCTQAVIFARVSTKEQEPGASLKAQK |
| recombinases | | 7180 large | | EAMEDYCNKKGLPIVKKYKAIESSTNGKRVQFNEMLD |
| | | serine | | FVRKQKQKTAIVVHCIDRFQRRFNECVEVESLLLDDKT |
| | | recombinase | | DLHFCKEGLILTKNSPSSDIMRWDMGILSGKMYVANL |
| | | | | RDNVNRGMNYNWSIGKYQTKAPVGYLNVNKDIVVEP |
| | | | | DRAPFVKKMFEMYATGLHSIKSLHNFAKEMNLCSSHS |
| | | | | KTNKPLGRETIYAMLKNPFYYGEMVIRGEKMPHAYEP |
| | | | | LIAKSLYDKVQELLSGGKIHTRTQEYAGIPFVFRGLVK |
| | | | | CAECGCTISSETHKKKSGRSYTYLRCGHTKGACNQEL |
| | | | | VSENLLLQQLDDEVFSKIRLSRNILEPLKKCVQKRLIEE |
| | | | | SDANMIMKRKITTELNNLEARERRIKDSFFDGDITREE |
| | | | | WQEEKANIVAKREELQRIAEKYADISKDIQITVNEVLDI |
| | | | | AATVSDIMKEANPTQQNKLLSLMFAECYLDGQKLIYK |
| | | | | LQKPFDKLVNLKSAGTWFDFDKSDIKEYETMAEKVQ |
| | | | | MYKIERTKYLG |
| MG178 | 7119 | MG178- | protein | MQKKIIDNKGKAVIYARYSSDRQREESIEGQLRVCEEF |
| recombinases | | 7181 large | | AEKNNLQVIETYIDRALTARTDRRPSFQKMIADCKKKQ |
| | | serine | | FEYILVYKLNRFSRNRYDSAVYKHKIAQYGVKVLSAM |
| | | recombinase | | ERITDDPSGILLESLIEGIAEYYSAELAENVHRGMKENA |
| | | | | LEGKANGRLPLGYQKGSDGKVIVDPATVHAVKAIFNG |
| | | | | TAAGKRMKAIAAELNEAGYKNAFGRPYVPSSFAAIIRN |
| | | | | KKYIGTYQWADTEVEGVLPALIDNATFEKANEVLDSR |
| | | | | KRKSNRVRSDQYLLTGRLVCGDCGASYVGKSGTGRL |
| | | | | GHPYPYYCCANRIKRKGCTAKNFRQDKLETWLAVETV |
| | | | | KALNHPEVIQQLSTQILDAQKRLKSEKDPLIDGLAAEIK |
| | | | | DYQKRLANSIKAIESGIFSKTISANIKDYEEKIKALEKQL |
| | | | | SRAKLKQQPFTLTADHIEFFLTALLQGNPEDQMYRNNL |
| | | | | LDVLVSRVVLYPNRAEVFYRYKKELPSLPNPVIIREERG |
| | | | | SNGTQLVGPLGFEPRTNRL |
| MG178 | 7120 | MG178- | protein | MKKAALLLRCSTDSQDYDRQQRDLLPTAESMGYEIIE |
| recombinases | | 7182 large | | DLIFGEYVTGKDDVRKKDRESIANLKQACKEGKVDAIF |
| | | serine | | INEVSRLSRDSIAGRLFIREFNDDYKVPVFFRDLQMWTI |
| | | recombinase | | DPNSRIKNTYLEQMLGFYFDAAAAELKSMKTRFASGK |
| | | | | RKNARTGKSIGGVPSIGYTKDKEGIIIIDEDTAKYIRIIFS |
| | | | | KYLEKEGTITTVGRYLRGISNIRTWGNGTIGNILRNRAY |
| | | | | TGKLEVSITNPDETDDSKKIEKFITTIPVIIDEETFNKVQQ |
| | | | | KLDSNRSTQEYTRSKVHLLQKLIICSDCGKAFSTKTVN |
| | | | | SGRNYTYCCVSKQNGINCSLPIALDADKTEAIIWNLVK |
| | | | | SKLIELNKLGEKEKEERIAKEKQQIQIYEEEITAIELGIAK |
| | | | | IARKKKNLIDYLEEAESDEEIADIKARRKKYDSEIAENK |
| | | | | TRINHLKEKIEICKSNINQHLNSSLTSSILSSIESDRNKMK |
| | | | | VQIKEFIKRIIPYPIANSHSVLEVCTTFGRYYILYSSRDK |
| | | | | YKQAYFLYGDYYRYFQKNNKFLVLGDMDEDTIEKNM |
| | | | | KVSAFARTFTVDGYSKPKNTDTSNDKENISLSDAIKQNI |
| | | | | YHFSKNNNIGVYTYEEMKEKCRKMDWVLPFTI |
| MG178 | 7121 | MG178- | protein | MKPKCFSYTRFSRPEQAEGDTLRRQDSMAFEYAEKHG |
| recombinases | | 7183 large | | LVLDKSLNMSDHGLSAYSGDNKKKGALGSFLKLIKDG |
| | | serine | | KIAKGSVLLIEHVDRLSREPFNDAHVQFNSILESGVDIV |
| | | recombinase | | TLSDSQHFNRQSINDIGSVIPTLIKMDLSHQESEKKSQR |
| | | | | LKAAWQNKRDNVSTKKLTSRGPAWLELSDDRSEFIAV |
| | | | | EERAKVIKMIFDMKLGGKGSQLIARTLNQDSELWKPST |
| | | | | KWRKSYIGKILRNRACIGEFQPMRRIEGVRQPVDQPVK |
| | | | | DYFPTVVQEETFLKVQALIKNNKFYGGRNGAISNLFSH |
| | | | | LMRCGYCQGPMRLIDKGKGRKKLICDNAVRGIGCERL |
| | | | | QIHYKNFEDIILDFCVGLKVSDLIKDDQHLIELEQLKDK |
| | | | | VLLIDDRLKTIEKEEAFFIKRMKKTLDDRIADDYEKGIS |
| | | | | ALKDEKEILKEDRSKAQTEIEKLSVLDADIKLRLSDIKD |
| | | | | LKEIMKNLKGDELRDIRLRLRDKIRDLIDSIDIFAKGDP |
| | | | | KETENLIEDMKGIYTEDELKDELKGDPNYVISFKSGVIR |
| | | | | SLFPYKDKKLSWQLDKTKKQLDIY |
| MG178 | 7122 | MG178- | protein | MQNNSGVLWVRVSSDDQAKGYSPDSQERLLESIAAKR |
| recombinases | | 7184 large | | EIIPVKKFNVTESAKTSENRKLFKEMIEFIKKNEIRNLVA |
| | | serine | | LSADRLARNYQDFTTLQILVDKHNVSIILAETNKIINQN |
| | | recombinase | | SDYSDRFLFQLLASLSEMGNRQRSADTRRGMEQKARQ |
| | | | | GGAPYYVPIGYLNVVDPQNPKRKFVIVDEERGSLIKKA |
| | | | | FELYDTGKYSLFTLADELNRLGLRTRPTKSHSAAPITKS |
| | | | | SIEVILKNKFYIGLVLQRGQYYPGAHQPLISKQLFDSVQ |
| | | | | GRLAQHCSYSRPDSKKVFPFKKFLKCGYCGCQLTGEE |
| | | | | QQGKNGNSQYRYYRCTFSKDRNCPQKYYREEEIDKML |
| | | | | TEAMGDLYVDETIAEEIRKRLKSTHLEQSSWEDKERAR |
| | | | | LQAAETKKTRHLDLIYEDRLNEIITPEQYKQKSNAIQGE |
| | | | | LAQIKSDINKLGKTNLKYKEEGSTILALLKGLKQTYEK |
| | | | | QDYQGKAEILALVLDKVFLRDGKAQFHWKPPFDFLFSI |
| | | | | NKILEEEEPQAGSFIRSSERDPDSGLKSICSISRKGRFSRG |
| | | | | IYPGSPLI |
| MG178 | 7123 | MG178- | protein | MTKRTAIYARYSTDLQNERSIEDQLALCRSYAERNGLL |
| recombinases | | 7185 large | | IVDTYTDAAVSGSSTVNRQGWLKLMRDAEAKRFDLV |
| | | serine | | LAEDVDRISRDEADYHTARKRLAFLGIEIWTAHSGKVT |
| | | recombinase | | GIEGSVRAMMASHYIENLAHKTRRGLAGVIKSGRHAG |
| | | | | GRAYGYRTVPGKPGELEIIPEEAEVVRRIFTDYVCGRTP |
| | | | | REIAHALNNEGTPPPRGKRWSASTINGNKARGYGVLQ |
| | | | | NELYAGRLVWNRVRMVRDPATGKRISRANPKSAWQT |
| | | | | QEAPHLAIVSREVFDAAQFRKAARSIGGGHKHRRPKRL |
| | | | | LSGLLKCGACGSGMSVFGADKTGKVRIRCTAATESNS |
| | | | | CPDPRTFYLLAVEETVVDGLRRELQDPKVLTEYARTYI |
| | | | | EERNRIAQRAAQDRGKLERKLAKVSGEYDRTLRLYQK |
| | | | | GVLSEEVAERKLPRLQAERDRLTAELEAEPVVENKITF |
| | | | | HPGTLARYEGALARLQTELEKGAAESDAEQAAAIREL |
| | | | | VETVTVRRDPSRRGGVEVEISGRLAALLNAPVYPGHLR |
| | | | | SPVGGNAGSGGGTRTPDTRIMIPLL |
| MG178 | 7124 | MG178- | protein | VRAATYERISQDRESTEHGVDNQRSANLALAARLGFD |
| recombinases | | 7186 large | | VVSDYRDNDTGASTRSRKARPGYAAMLAAAKRGEFE |
| | | serine | | AILAYSNSRLTRRPREFEDLIELHEQYGIRIVTVVSGDD |
| | | recombinase | | DLSTADGRMVARIKAAADAAEAERTGERVAFAQAAK |
| | | | | LRRGEDIGGRRPFGFEADRITIRESEATLIREGVRMILGG |
| | | | | ASLYAVARAWDAAGIREKPWRSQTVRDILTRPRNTGR |
| | | | | LVVGGVEYGRGDRPAILTDEEYADLLAVLRTNERPRR |
| | | | | GRKPQTSTAVSVVRCGVCGAGVELTIKSGGVRTIRCSV |
| | | | | RGGGQRHPTMTDDRLEMQLAQVALMRVVDPFATENA |
| | | | | DRPEVAKLRRTLADVTTRRDRAREDVEAYDDPDDRA |
| | | | | HARKRVTELTAAVREARAALDAALAENVASRARHLV |
| | | | | DVAHKRLGGEIVAVDPFQVWPQWVELWRSWPVADR |
| | | | | RELLRGRLIELMPHVRGESWRLRVDGKRPAGATQREG |
| | | | | EPI |
| MG178 | 7125 | MG178- | protein | MAKGNRAAIYARFSSHNQRDESIEIQVEKSREYCEQRG |
| recombinases | | 7187 large | | LDVVCVYSDYAQTGRNTARAEFQKMMDHAKLGMFD |
| | | serine | | YVVIYKVTRIMRNRDEMALARIMLRQAGVEILYAGET |
| | | recombinase | | LGHGSTRVLHLGMLEVLAEYESAVDSERIRDGIQKNAE |
| | | | | RGMANGQTRYGWDIVDGYYQVNEQEAALLRRMKNM |
| | | | | LLSGSTLAEITRAMEGERTRAGKKFTIKGITKLLRRWQ |
| | | | | NCGVYEYAGVRIEDGMPALWTREEQEMLIRILTHRTIT |
| | | | | QRRRGEAEEYPLSGKLFCRECGRYYSGTCGTSKSGTRY |
| | | | | FYYRCPSCRRAFRSGLIEAWACDAVFEATKGKHFKEQ |
| | | | | LADAMAVFDESSNGAHEIEAKRLRKEISKIDAAFERIW |
| | | | | KAIEDGCAPPGGKDRIADLKTRKAALEEDLAQAEAVSS |
| | | | | CENISKEDLEEWIGLIAKENDTKKIIDRFVRFIEFDGQEG |
| | | | | HIYFTFDHHGNDFMPTKKANTQMKGCSPIESMVDRRG |
| | | | | LEPRTLGLRVPCSTN |
| MG178 | 7126 | MG178- | protein | MQNLPSGEYWMYLRKSRADLEAEARGEGETLKKHER |
| recombinases | | 7188 large | | MLYKLAKDLGILITEEPFREIASGESIYHRPEMLRMLDL |
| | | serine | | MEERRPKGILVMDIDRLGRGDMQEQGLILGTFQRLNIL |
| | | recombinase | | IITPRKIYDLNNEFDEEYSEFEAFMARKELKIITRRLQRG |
| | | | | RVLSVEAGNYIATRPPYGYQVIKDGRNRYLVPHPEQAP |
| | | | | VVKLIFELYTHDDPEKRMGSNKIAIKLNELGYTSYTGK |
| | | | | KWTSSSVLTIIKNAVYIGRIQWKKKEVKKSKITGKKKD |
| | | | | VRTRPVQEWIDVQGKHEPLIDEVTFQKAQEILKQKYH |
| | | | | VPYQQLNGITNPLAGVIKCAKCGASMILRPYTKQAPHL |
| | | | | MCYNRFCDNKSSQIAYVEEKLLQALEKWMDTYVIEYG |
| | | | | QRKRKVSNMVEVKQNAVNLLKREMDELEAQKERLHD |
| | | | | LLERGIYDEETYLDRSKKLAERISSTKERIERAEQELKE |
| | | | | EQHKEQAQKDVIPKLKNVIKLYWKSKYPAKKNALLKS |
| | | | | VLLHATYKKEKWQRKDQFELVLVPKFK |
| MG178 | 7127 | MG178- | protein | MKKEIKEEPKKKAVVYARYSSHRQGEQSIEGQLAEAY |
| recombinases | | 7189 large | | KYAAAHGIKIIHEYIDRAMTGRNDNREQFQKMLRDTA |
| | | serine | | KKQFETIILWKIDRFGRNREEIAFNKYRCKKNGVKVVY |
| | | recombinase | | VAESIPDSPEGVILESVLEGMAEYYSLQLSQNIRRGQRA |
| | | | | SAEKCQCTGGNRPLGYRTDPKTKKFVIDKETAPTVKMI |
| | | | | FEMYANGIPLADIIRTVNNKGLRTLRGNKFNKNSFKRL |
| | | | | LKNEKYIGVYKYKDDILVEGGIPAIIDKETFERVQEML |
| | | | | KKNQTARSAKGKKADFLLTDKIFCGRCGEPMIGESGV |
| | | | | GKTGKVYYYYTCTDRKNKKQACKKKPVPKNKIEKLVI |
| | | | | NKIAEILHNEDLLDMIIDKVYAVYREEHNNDDERIMLN |
| | | | | KKLAEIHLAQENILKAIEQGMISPLFKDRTAELTAQQSE |
| | | | | IESELASIEAQEKIQLTKEHIRFFLEDLSSKDIDDTDVQK |
| | | | | KLIDTFLNAVFVYDESVTFAFNYSNNGEKVTLSEVDNI |
| | | | | NGSGDFFECGYDGGDDETRTHYLYNANVALSQMSYA |
| | | | | PKRL |
| MG178 | 7128 | MG178- | protein | MFSDFGNELKYAVKYTRVSTNQQDDRGSKEIQDLKIN |
| recombinases | | 7190 large | | EFADKNHFKVVNSFTDTDHGDNPLRPGINALKSYLKA |
| | | serine | | NGEVKYVICLFQDRFTRDFREGLENLYFLKDLGVSLIT |
| | | recombinase | | VNEGLIKMDGTFDSIPALIRFIGAQEEKTKIVKKTTDSM |
| | | | | YNYANTNRFLGGSILPWFKLEKVSENGKRIKIIVKNEET |
| | | | | WNIYRKFFIDIIRMKSVKKAALENNLNPYTVRDWVKM |
| | | | | PELIGYRTYGKKGKINNTYKKGKRAEYMVTSEKVLPSI |
| | | | | LSEEEYAKIDSVYKTYKVKFTSSRFPYLFTTLLHCECGG |
| | | | | RYFGNSLKNRYNTYYHYYKCEKCAKRYNAKNIEQEII |
| | | | | DAILENKNLNMLNDYNFRIADLYDQITILNKKIEIEKAK |
| | | | | ENNIVELMLEGIISNDISKEKLRTLKNNITNIEKEKKKLE |
| | | | | EQIEIESNKEITEEHIESLKFLLKNYDEETVSELKEILNLII |
| | | | | QKIVLSKNGEIEVIF |
| MG178 | 7129 | MG178- | protein | MNAVIYARFSSDRQNEASINAQVRACTEYAERHDLTV |
| recombinases | | 7191 large | | TGIYADEAISGKESKTAARAQYQKMLRDAHKGLFSVIL |
| | | serine | | IHKYDRVARSLAEHVNLEGRLKADNIELVAVAQDFGN |
| | | recombinase | | TSEAKIMRALMWSMSEYYLDNLSAEVQKGHRETALK |
| | | | | GLHNGGYAPFGYDVVNQQYVINELEAAYVRRIFTAAQ |
| | | | | EGTGFKHIIAELEAAGITGKRGKPIKYTQIYEMLRNEKY |
| | | | | TGVYLYTPQEAVERAQRRQKPEAIRVEGAIPAIVTKAQ |
| | | | | FMEVQAIMNSRKNAGRKADYMCSGLVYCSCGAKMH |
| | | | | ACKSTRKGHTYYRYVCSEHCGRPTVLMSAVDEAAIRY |
| | | | | LRELLSDPNQMLITAAMRRYQSDSKNRLTAFYDVLNA |
| | | | | RIAEKQKEYDTLLKNLSSGVLPSDVVADIGQRMTEIKD |
| | | | | EIKALEATEPPEDYTVDTITRWLNALKNNPDEKAVKLL |
| | | | | VKRIDVSGDKKNNVFNIQSTLNTLLEIMVAETGFEPAT |
| | | | | SGL |
| MG178 | 7130 | MG178- | protein | MTLQKARIGGETTKAVIYARYSSHSQREESIEGQLREC |
| recombinases | | 7192 large | | HEFALKNGFTIINEYIDRAISGKTDNRPSFQRLIKDSEKG |
| | | serine | | QFEAVIMYTLDRFARNRYDSAIYKAKLKKNGVRVYYA |
| | | recombinase | | KQPMPDTPEGIILESVLEGYAEYYSENLARNIKRGIREN |
| | | | | ALQGLATGGANLLLGYTVGEDRKYAIDPTGAKIVQEIF |
| | | | | QLYADGMSATQIIAYCNERGYKTARGNAFNKNSLRTIL |
| | | | | RNEKYIGTYKLMDIVIPDGMPAIIDKVLFEKVQAMLKH |
| | | | | NGKARAKAKAHENYLLTTKLFCGHCGSPMVGESGTS |
| | | | | KTGQVHYYYKCTKAKREHACKKKSERKDWIEKLVVR |
| | | | | YTVQNVLTDENIALIAKRAMEIIEKESADTTYLDGLNA |
| | | | | ELKDVQKKIKNLVSAIEQGIITSATKDRLDELEQEKSDV |
| | | | | EGRIAREEMKKPLLNESRIRYWLTSFKSGNVDDEDYQR |
| | | | | RVIDTLVNSVYVYDDEDGGKRIMLTFNLSGNNTATLTS |
| | | | | SDIGCYAPPKEIRRNTVSAVFLYNRPTVFVPVPHLQRR |
| | | | | HHEKETDRKRVRLCCQHAVWLVLRRR |
| MG178 | 7131 | MG178- | protein | MLSKNACAYIRVSTDKQEELSPDAQKRLILEYCKKNNL |
| recombinases | | 7193 large | | TIMSEHIFVENGISGKKADKRPQFQRMIALAKQKEHPF |
| | | serine | | DVILVWKFSRFARNQEESIVYKAMLQRAGVEVVSISEP |
| | | recombinase | | IIDGPFGSLIERIIEWMDEYYSIRLSGEVMRGMTEKAMR |
| | | | | GGYQSTLPLGYHMNPDTGVPEIYEPEAAIYRIILDRYLN |
| | | | | AGMSPLAIARELNAAGYRTRRGAPFERRTIIYILENPFY |
| | | | | TGMIRWNRQNHSDHTIKDRSEWIFAKGAHAPLIDKDT |
| | | | | YDHIQAVTALRTRPYKARGTSSIKHWLSGIVKCSDCGK |
| | | | | SLVGNALYNGVPSSWQCSDYNKGRCCHSHFIKNTALE |
| | | | | QAVFNALEHAITSGDISYTLKSTDSSSDLASDLDRMLS |
| | | | | KIDDKERRIKQAYRDGIDSIEEYRENKEILTKERQDILSR |
| | | | | LAALSERSSEDDKKKLLAEVQSVYDIVTGNADKLTKA |
| | | | | NAIRSIVDHCVYDKANDSFDIFFFFSKGSEPL |
| MG178 | 7132 | MG178- | protein | MVFASYTRKSIYSDKSDSTKNQAKMCRDYVDFHYAGS |
| recombinases | | 7194 large | | VDSFLVYEDEGLTGSNTKRPDLQRLMKDIKSGLIDFLIV |
| | | serine | | YQLDRLSRDIKDFSNIYAFLEEHHVQFISVAENIDTNTPI |
| | | recombinase | | GKAMMYVSVIFAQMERETIANRVNDNMIGLADDGW |
| | | | | WVGGNPPYGWRRTRITSSDGKNHVTIVAEEEEAEFVIS |
| | | | | VGRIFLQNNFSLQQLERYFKNHNILTKNGKFFSTNQLH |
| | | | | KLLTMPYCAPATQAIYDYYSNLGCIMSSRCPRELWDG |
| | | | | THGVMIYGRTTERNKKHALQPPNKWRVCIGRHKPFM |
| | | | | DESTWLSIQNQFTHNVFDKKMKHPIPLLKGVLRCKCG |
| | | | | RLMMLARKAKVDGSVSTWYYCPKRMRQGADYCDMS |
| | | | | QIKTDIIDDKVIDIFNKISKDPDTINEYLLVSVPVKDFNT |
| | | | | EIKDANKSIIKIKNKIQNLTNALAETPDTSAAKYILNTID |
| | | | | GLDKNLKLTERHIADLQSQERKSSQTELEVYEKQRKIT |
| | | | | DFIRNFENFTPEERNAIARTCIKECVWDGHTLSVVL |
| MG178 | 7133 | MG178- | protein | MLNVVIYARFSSSAQREESIEDQLRECQEFADKEGLQVI |
| recombinases | | 7195 large | | NTYCDYAISGKTDHRDQFQKMIKDAEKQLFQAVIIYKT |
| | | serine | | DRFARNRLDSAIYKKRLKDCGVKVIPAKEVIPDGPGGII |
| | | recombinase | | LESIYEGWAEMYSVNLAENVRRGQHGNALKCKANCK |
| | | | | APFGYKINPQTRLYELDDTTAPIAEKVFAMAAEGKPTK |
| | | | | DLQKFLLSNGIKKSASYIHYMLRNERYKGIYIFDGVRV |
| | | | | DGGMPKLVSEDTFTKIAKTLKQRSIRPQANAAKYFLSL |
| | | | | KLYCGYCGKLMSGEYGRSRNGDQYRYYTCPSSRRKK |
| | | | | TCELKALPADKTENTIAEKLQTTLLSDEIIDTMADYIID |
| | | | | YQRQVYENDSMEKALSKQLSDVEKRINNLLAAIEAGA |
| | | | | MTDSTVNRLHDLETKQKELLTSLSIEKLKAPIITKEKIV |
| | | | | YYIKKYRDNDITVNEIRQEFLNTFVSKAYVFSDHLFVIY |
| | | | | DAINGINTEVTPEILSNPNEFGYIPIWWN |
| MG178 | 7134 | MG178- | protein | MNAVIYARFSSDKQSEDSIEAQVRACREYAAKHGFNV |
| recombinases | | 7196 large | | LSVYADEAISGKTANRAQYQKMLRDCNKGLFDTILIH |
| | | serine | | KYDRIARNLGEHVNLEMKLKEKGITLIAVAQDFGRSKE |
| | | recombinase | | AKIMRALMWSLSEYYLDNLSSETKKGHKETALKGLHN |
| | | | | GGYAPFGYDVVNQTYIINELEAGYVKRIFDAALNREGF |
| | | | | TSLIEEMDKAGIRGKRGKPIKYPQIYEMLRNEKYTGVY |
| | | | | TYSQEEETNRSDRRNKPHAIRIENALPVIISKAQFMEVQ |
| | | | | KIMNKRKQTGRKGNYLCSGLVYCECGAKMHGMTSKR |
| | | | | KGHEYRYYTCSKHCGAPVVRADDVEQAAYRYLYTLL |
| | | | | SEENQTRIADALRQYQAGEGSRMDEFKQALAKRIQEK |
| | | | | QNQYKALLANLSTGALPAEIVADIAAEMKDIKEEIALL |
| | | | | ERTEPPKDFTVDQIRAWLEALKATPDDKAVRLLISRIDI |
| | | | | KQKTIINMESTLTMVLSEIGCGSWI |
| MG178 | 7135 | MG178- | protein | MQKTDNKMRAVIYARYSSDRQREESIEGQLRVCEDFA |
| recombinases | | 7197 large | | QKNDLMVVDTYIDRALTARTDRRPAFQKMIADCKKR |
| | | serine | | QFEYILVYKLNRFSRNRYDSAVYKHKIAQYGVKVLSA |
| | | recombinase | | MERITDDPSGILLESLIEGIAEYYSVELAENVLRGMKEN |
| | | | | ALEGKANGRLPLGYQKGPDGKVIIDPSTAPAVKLIFKG |
| | | | | TAAGKRMKVIAEELNAAGYKNASGRPYNPNSFAALIR |
| | | | | NKKYIGMYQWADTELEGVIPALIDKATWEKANGVLNS |
| | | | | RKHKSNRIRSDQYLLTGRLVCGSCGSAYVGKSGTSHR |
| | | | | GTTYQYYCCSNRIKRKGCKGKNFRQDQLESWLAAETI |
| | | | | RTLNHPDIIRQLSNQILAVQKSLETEKDPLIDGLSAELKE |
| | | | | YQKRLANSIKAIESGIISDTISANIQQYEEKIKILEKQLAR |
| | | | | AKLKQQPFTLTASHVEFFLSALLDGDPKDQEYRTKLLD |
| | | | | ILVSRVVLYPNKAEVFYRYQKELPSLPNPVIIREERGSN |
| | | | | GNQLVGPLGFEPRTNRL |
| MG178 | 7136 | MG178- | protein | MNAVIYARYSSDKQTEDSIEAQVRACQEYATKNNINII |
| recombinases | | 7198 large | | GVYADEAVSGKTANRAQYQKMLRDCDKGTFDTILIHK |
| | | serine | | YDRIARNLGEHVNLEVKLKDKRVTLVAVAQDFGTSKE |
| | | recombinase | | SKIMRALMWSLSEYYIDNLAAETRKGHRETALKGLHN |
| | | | | GGYAPFGYDVVNQTYVINELEAAYVKRMFNAALNRE |
| | | | | GFTELIEEMNRAGIRGKRGKPIRYPQIYEILHNEKYTGV |
| | | | | YVYTQEEESDRGNRRAKVNAIRKENALPVIISKAQFME |
| | | | | VQQIMKQRKHSGRKSNYLCSGLVYCECGAKMHGMTS |
| | | | | KRKGHEYRYFTCSQHCGAPVIRMEEVDEAAYHYLHTL |
| | | | | LSEENQDRIADALRLYQAGEGSRMTEFKQVLAKRIREK |
| | | | | EEQYQSLMENLSSGILPKEVVSDIAERMQQIKEEIAVLE |
| | | | | ATEPPKDFTVEQIHSWLEALKAAPDDKAVRLLVSRIEV |
| | | | | KQKTVFNIASTLKAVLCETGCGSWI |
| MG178 | 7137 | MG178- | protein | MENNSAVIFARVSSKEQFERWSPKIQEEAAAKYAEAH |
| recombinases | | 7199 large | | KLDVVRVWNIAESGYKSRKEFQAMLAFIKGNSVRHLI |
| | | serine | | TMNSDRLTRDLRGMLDVDKMIQDENLSVHFIESNEIID |
| | | recombinase | | ANTTRSQKSLWKIKVIFAENYIGDLQEKVRRSMEARLD |
| | | | | IGLFPFINPPFGYDFKKNRLTPNGNADVVRKAFSLYAG |
| | | | | GTESAFSLMKKLKAEGLRLSEPAVNNLLHNPVVAGLL |
| | | | | VWPWDNSKYVKTEHFRNELIQGQQEAIVDRETFERVQ |
| | | | | EILKRKTHSHPFKKDQIFFQYRGLIRCACGKLLSGAQFG |
| | | | | KIVYYAPKHRDSSCGEKPVRSEVIDKAVAQALKGFSFP |
| | | | | KDLYDWARDVLRTTREDTKSHAQAERRKAQTEYSLTI |
| | | | | KELDVAFSSAVTGIFDVVTVRRNVMEIKERQEKAKAV |
| | | | | LNGLDRSDRKFVDDGLAILELLKNVEKAYSQAMPEHR |
| | | | | AELLRVLFEDISISGGKFVFTPQAVFAPLFDLRHGKPRQ |
| | | | | AAIRLAVGVPDLPEHGR |
| MG178 | 7138 | MG178- | protein | MNMSAGPRAVIYVRISVAQEASVSIERQVEAAEQYAA |
| recombinases | | 7200 large | | ARGWQVVATFRDEGVSATHNKPEDRAGWRALLDSPE |
| | | serine | | KYDAVLVWKIDRLARRVLDFLHADASLQERGAGIVAV |
| | | recombinase | | EDPVDMTTPQGRAFATLLAVFGEMEGEAIRARVKAAR |
| | | | | DHLLRARRVVGGTVPYGWRKVANPDGPGYVLAQDPE |
| | | | | RVGWVRGMVERAQAGASVYSIVQWLDEAGAPLPEAS |
| | | | | QSRRKAGGWSYSTVERLLRNPLVAGMTAYNPGNRTK |
| | | | | ERGADVLRDADGLPVVDESVALLSPGEWRALVKALD |
| | | | | ERDTAQSKPVALRAKTSALLSGLLWCEADGTRLHRGT |
| | | | | INGRHGYYCPECHQSISNFEDALVAEFLRQKGEHVRWS |
| | | | | VVEEVYEGGAAVLPEIEHRLAELSDALRATDDDAEAD |
| | | | | RLMEQIGNLRAIRREARGKAPKVEYRPVRGTQRFGDD |
| | | | | WADAETVEDRRAILEDGLERVWVSRGRPGRTTDAQRL |
| | | | | ARLRFDWKQPEHLGPLARPSDAELAAWAE |
| MG178 | 7139 | MG178- | protein | VIPIEVEKKRLGACYIRVSTDDQTEYSPDSQERLIREYA |
| recombinases | | 7201 large | | EKNNIFIPDEYVFRESEGISGRKADKRPEFQRMIGAAKQ |
| | | serine | | KPAPFEIILVWKFSRFARNQEESIVYKSMLRKQCGVDV |
| | | recombinase | | VSISEPLMEGPFGGLIERIIEWMDEFYSIRLSGEVKRGM |
| | | | | TERISRGKPTNAAPFGYRWGNDNYEIVPEDAELVRQIF |
| | | | | ERFINGESYLSIARWLRTVCDKRDWENRTVEYILRNPA |
| | | | | YIGMLRAGIAGEKNNSRDFYGSNMKLYQGTHQPILSPE |
| | | | | TFQAAQKRADHIKNTHPKWDHSARAVPRLWTGILKCS |
| | | | | NCGGTLSSGGKNDSWQCVRYLHGKGCGVSHYTTTKA |
| | | | | VSEALLPVIIQDLKTGKNLEKLDLGQHTKSSESQDVIKQ |
| | | | | IDRLKARLKRVREAYEAGVDTLAEYKASREAIEKEIET |
| | | | | LQHKVDKAPNAADIEKLHQKLMEKNQRYIKLLQDEK |
| | | | | ASDAEKNEALHHIVDKIVFQRSTNHFDVFYSESYML |
| MG178 | 7140 | MG178- | protein | MSSLITGECKISARVYSYLRFSDPRQATGSSADRQLQY |
| recombinases | | 7202 large | | AQRWAAERGLVLDESLSLRDEGLSAYHQNHVKQGAL |
| | | serine | | GAFLRAVDEGRIPDGSVLIVEGLDRLSRAEPIQAQAQL |
| | | recombinase | | AQIINAGITVVTASDGREYNRAGLKAQPMDLVYSLLV |
| | | | | MIRAHEESDTKSKRVKASIRRLCEGWVAGTYRGLIRN |
| | | | | GQDPQWLRWDGQAWHLIPERVEAVRYAIELYKQGEG |
| | | | | ATRAARKLAERGYVLSDWGIAGQQIYRLVKLPALRGA |
| | | | | KRISVDGEDYLLEGYYPPVLTDEEYEALQAATETRHGR |
| | | | | RGAPEIVGLVTGLGIAYCGYCGTAVVAQNLLSRARKD |
| | | | | GTVADGHRRLHCTSYSKSTGCKAASCSVVPVEKALLS |
| | | | | YCSDQMNLTRLLEPADDGQQLRQRLQACRRKQADVE |
| | | | | RQLQRITEALLADDQGAAPLAFVRKARELEAQLGQLQ |
| | | | | AEAEHLEREQGKVGQTQTPAGAELWRKLAIEAQDIKS |
| | | | | PAREQLRQLVLDTFSRITVYMRGLVPDPKSKVIHLVLV |
| | | | | SRSGQRVVLDVDRRSGAWKAGRDRRG |
| MG178 | 7141 | MG178- | protein | MKTAVAYARYSSDNQRDESITAQLRAIREYAAKNGIEI |
| recombinases | | 7203 large | | VREYTDEARSATTDDRPGFQEMIRDLKNGLKVDLVLV |
| | | serine | | HKLDRFARNRYDAAVYRREIQKAGARLVAVDQPLDD |
| | | recombinase | | SPEAVLLESLLEGLAEYYSRNLAREVMKGLKENALKG |
| | | | | LHTGGRPPLGYRLENGRLVIEPREAEAVRLIFQGVLDG |
| | | | | KSYTAIQQELNAKGYRTREDRPFGKNSLSDILRNEKYT |
| | | | | GVYVYNRTARKVAGKRNHHASKPPEEVIKIPGLIPAIIT |
| | | | | REEWDKVQEILNQRRKVRPRKRGETEYVLTGKLVCGV |
| | | | | CGSAMVGNSKRNGKGTVYRYYECNKAQRTGECTNRP |
| | | | | IGQKVLEQIVVQQIEEDILSNPEELAEQMAAYHAERGG |
| | | | | YLKRKKMALKARLAACQEKIDKVIDRLIEIGREEELLR |
| | | | | KLNELKAEREALQKEYAALPEEEPPVTKETALEYLNH |
| | | | | VSEALKEAKSPAEYRAAIHRFIDRIVVGEKMIQIHFLAD |
| | | | | FGGGVWIKLVELEGFEPSTS |
| MG178 | 7142 | MG178- | protein | MKRIAIYSRVSTADKQDYTRQVNELKKIGYDNGFSDK |
| recombinases | | 7204 large | | QMTLYSEAISGYKKDERHQLNAMLSQIEADPTYFSAV |
| | | serine | | YVSEISRLGRNPKETRRIVDRLSELKVTLYIQSLKRYTL |
| | | recombinase | | DDKGAMSIDTSIILQVLMEYANLEAETFKTRSRSGLRK |
| | | | | AAMDGKYIGGVASAYGYTQDKNNYVIIDDFEADIVRM |
| | | | | IFTMYSEGQGTKKIANVLNTRKIPTKYNRIFEGTKRIIK |
| | | | | GSLKEATKINWSDAVVYAILKNTSYIGEKKYKGEIIATP |
| | | | | AIIDKKLFNKCQEIMSGKSHRNYLTNYTFLLKDLITCTC |
| | | | | GRNFYARYKPVEGGDKVYVCSSRLVRGGRCACPNGIN |
| | | | | ITLLESAIYDYLVTHSRYLTNMASVDKMKAKLWQEIK |
| | | | | SLTDKANIDQKAMKAKANEEKRLLDVYVSGAITKAEF |
| | | | | EAKKKKIKQEISVISEREKILEKDINEKNEAFKRADSGN |
| | | | | APLNALEKAKDNRNELQALYRQILTKVNVESTTKETA |
| | | | | VVSIVINATKAVMIELDLSGIRRKPMQYQYRTGKDWV |
| | | | | AIPDKRLISL |
| MG178 | | MG178- | nucleo- | GGGCAACC |
| conserved | | 7202 2 core | tide | |
| core | | | | |
| MG178 | | MG178- | nucleo- | GGGCACCC |
| conserved | | 7202 1 core | tide | |
| core | | | | |
| MG178 | 7145 | MG178- | nucleo- | AGTATGGTGGAC |
| conserved | | 7193 core | tide | |
| core | | | | |
| MG178 | | MG178- | nucleo- | CAAGTTC |
| conserved | | 7177 core | tide | |
| core | | | | |
| MG178 | 7147 | MG178- | nucleo- | TTCATTTGACATCC |
| conserved | | 1859 core | tide | |
| core | | | | |
| MG178 | | MG178- | nucleo- | ACCGCC |
| conserved | | 7201 core | tide | |
| core | | | | |
| MG178 | | MG178- | nucleo- | CATATGT |
| conserved | | 7173 core | tide | |
| core | | | | |
| MG178 | 7150 | MG178- | nucleo- | AACTGGTTGCGGGAGCTGGATT |
| conserved | | 7198 core | tide | |
| core | | | | |
| MG178 | 7151 | MG178- | nucleo- | CCGATGGCGTCCAGCGACTCGGCCTTGGCACTGTCG |
| recombinases | | 7202 AttB_1 | tide | ATGAAGCCACAGGTGTTGACCACCACCACGTCGGCA |
| attachment | | | | TCCTGGTAGGTCGGCACGATCTCGTAACCTTCCATGC |
| sites | | | | GCAGCTGGGTCAGGATGCGTTCGGAGTCGACAGTTG |
| | | | | CCTTCGGGCACCCAAGGCTGACGAATCCGACTTTCG |
| | | | | GGGTGGCGGTGGACATGCGGGCTAACCTCTAAGGGC |
| | | | | GCCTGGCTGGCGCCTCTGATCAAAAAGTGCGCAATT |
| | | | | CTAGCGATGGATCATACGCTTGACCAGTGCTGTAAA |
| | | | | AGACGCGCAAACGAAAAAG |
| MG178 | 7152 | MG178- | nucleo- | CAGATCCGCAGGGCTCACCTGCGAGCGCGTCTATGA |
| recombinases | | 7202 AttP_1 | tide | CGGAGTGAAGGCAGTAGCTGAGCAAAAATGAAAAG |
| attachment | | | | GCCCGGTGCCGTGGGGGAGTCCCTGCGGCACCGGGC |
| sites | | | | CTTTTTTTGTTCCTGCGATTTTAGTTTAGAAAATGTA |
| | | | | GCCTATGGGCACCCCAAGGAAATAAACCCTACACTT |
| | | | | GGAGCTTTCTTAGTTTGAGTAGTTGTCATCACTGATC |
| | | | | ACGGGGGAATGCAAAATTAGCGCGCGAGTATACAG |
| | | | | CTATCTGAGGTTTTCCGATCCCCGGCAGGCGACCGG |
| | | | | CAGCAGTGCCGACCGTCAGC |
| MG178 | 7153 | MG178- | nucleo- | CCGATGGCGTCCAGCGACTCGGCCTTGGCACTGTCG |
| recombinases | | 7202 AttL_1 | tide | ATGAAGCCACAGGTGTTGACCACCACCACGTCGGCA |
| attachment | | | | TCCTGGTAGGTCGGCACGATCTCGTAACCTTCCATGC |
| sites | | | | GCAGCTGGGTCAGGATGCGTTCGGAGTCGACAGTTG |
| | | | | CCTTCGGGCACCCCAAGGAAATAAACCCTACACTTG |
| | | | | GAGCTTTCTTAGTTTGAGTAGTTGTCATCACTGATCA |
| | | | | CGGGGGAATGCAAAATTAGCGCGCGAGTATACAGCT |
| | | | | ATCTGAGGTTTTCCGATCCCCGGCAGGCGACCGGCA |
| | | | | GCAGTGCCGACCGTCAGC |
| MG178 | 7154 | MG178- | nucleo- | CAGATCCGCAGGGCTCACCTGCGAGCGCGTCTATGA |
| recombinases | | 7202 AttR_1 | tide | CGGAGTGAAGGCAGTAGCTGAGCAAAAATGAAAAG |
| attachment | | | | GCCCGGTGCCGTGGGGGAGTCCCTGCGGCACCGGGC |
| sites | | | | CTTTTTTTGTTCCTGCGATTTTAGTTTAGAAAATGTA |
| | | | | GCCTATGGGCACCCAAGGCTGACGAATCCGACTTTC |
| | | | | GGGGTGGCGGTGGACATGCGGGCTAACCTCTAAGGG |
| | | | | CGCCTGGCTGGCGCCTCTGATCAAAAAGTGCGCAAT |
| | | | | TCTAGCGATGGATCATACGCTTGACCAGTGCTGTAA |
| | | | | AAGACGCGCAAACGAAAAAG |
| MG178 | 7155 | MG178- | nucleo- | CCGATGGCGTCCAGCGACTCGGCCTTGGCACTGTCG |
| recombinases | | 7202 AttB_2 | tide | ATGAAGCCACAGGTGTTGACCACCACCACGTCGGCA |
| attachment | | | | TCCTGGTAGGTCGGCACGATCTCGTAACCTTCCATGC |
| sites | | | | GCAGCTGGGTCAGGATGCGTTCGGAGTCGACAGTTG |
| | | | | CCTTCGGGCAACCAAGGCTGACGAATCCGACTTTCG |
| | | | | GGGTGGCGGTGGACATGCGGGCTAACCTCTAAGGGC |
| | | | | GCCTGGCTGGCGCCTCTGATCAAAAAGTGCGCAATT |
| | | | | CTAGCGATGGATCATACGCTTGACCAGTGCTGTAAA |
| | | | | AGACGCGCAAACGAAAAAG |
| MG178 | 7156 | MG178- | nucleo- | CAGATCCGCAGGGCTCACCTGCGAGCGCGTCTATGA |
| recombinases | | 7202 AttP_2 | tide | CGGAGTGAAGGCAGTAGCTGAGCAAAAATGAAAAG |
| attachment | | | | GCCCGGTGCCGTGGGGGAGTCCCTGCGGCACCGGGC |
| sites | | | | CTTTTTTTGTTCCTGCGATTTTAGTTTAGAAAATGTA |
| | | | | GCCTATGGGCAACCCAAGGAAATAAACCCTACACTT |
| | | | | GGAGCTTTCTTAGTTTGAGTAGTTGTCATCACTGATC |
| | | | | ACGGGGGAATGCAAAATTAGCGCGCGAGTATACAG |
| | | | | CTATCTGAGGTTTTCCGATCCCCGGCAGGCGACCGG |
| | | | | CAGCAGTGCCGACCGTCAGC |
| MG178 | 7157 | MG178- | nucleo- | CCGATGGCGTCCAGCGACTCGGCCTTGGCACTGTCG |
| recombinases | | 7202 AttL_2 | tide | ATGAAGCCACAGGTGTTGACCACCACCACGTCGGCA |
| attachment | | | | TCCTGGTAGGTCGGCACGATCTCGTAACCTTCCATGC |
| sites | | | | GCAGCTGGGTCAGGATGCGTTCGGAGTCGACAGTTG |
| | | | | CCTTCGGGCAACCCAAGGAAATAAACCCTACACTTG |
| | | | | GAGCTTTCTTAGTTTGAGTAGTTGTCATCACTGATCA |
| | | | | CGGGGGAATGCAAAATTAGCGCGCGAGTATACAGCT |
| | | | | ATCTGAGGTTTTCCGATCCCCGGCAGGCGACCGGCA |
| | | | | GCAGTGCCGACCGTCAGC |
| MG178 | 7158 | MG178- | nucleo- | CAGATCCGCAGGGCTCACCTGCGAGCGCGTCTATGA |
| recombinases | | 7202 AttR_2 | tide | CGGAGTGAAGGCAGTAGCTGAGCAAAAATGAAAAG |
| attachment | | | | GCCCGGTGCCGTGGGGGAGTCCCTGCGGCACCGGGC |
| sites | | | | CTTTTTTTGTTCCTGCGATTTTAGTTTAGAAAATGTA |
| | | | | GCCTATGGGCAACCAAGGCTGACGAATCCGACTTTC |
| | | | | GGGGTGGCGGTGGACATGCGGGCTAACCTCTAAGGG |
| | | | | CGCCTGGCTGGCGCCTCTGATCAAAAAGTGCGCAAT |
| | | | | TCTAGCGATGGATCATACGCTTGACCAGTGCTGTAA |
| | | | | AAGACGCGCAAACGAAAAAG |
| MG178 | 7159 | MG178- | nucleo- | CACGTAACATGTAACAGCCGTTTTGACTGGATTGAC |
| recombinases | | 7193 AttB | tide | GGGCCTTGGCCATGGGAAGGAGGAATGGAATAATG |
| attachment | | | | TGGGTTTATGAAAAAAGACTTCAATTTCCGGTAAAC |
| sites | | | | ATCAAGCGGCCCGACCCGAAGGCGGCACAGATAAT |
| | | | | CATCAGCCAGTATGGTGGACCGGACGGTGAAATGGG |
| | | | | TGCCAGCATGCGTTACCTCTCACAGCGCTATTCAATG |
| | | | | CCTGACAATAAAGTTGCCGGACTGCTTACGGATATC |
| | | | | GGCACCGAAGAGCTGGCGCATCTTGAGATAGTTGCC |
| | | | | ACAATGGTTCACCAGCTTACCAGAA |
| MG178 | 7160 | MG178- | nucleo- | CGATAAACTTACCAAAGCTAATGCTATCCGGTCCAT |
| recombinases | | 7193 AttP | tide | TGTTGACCACTGCGTCTATGACAAAGCAAACGACAG |
| attachment | | | | TTTTGATATATTTTTCTTTTTTTCTAAAGGCTCCGAAC |
| sites | | | | CCCTATAAAATAAGGCCTTATCATAATTATACCTTAC |
| | | | | GGAAGTATGGTGGACTATTCTTCCGTAAGGTATAAT |
| | | | | ATCATCACAAAGCCTTTATTTAAAGCAATTACAGGC |
| | | | | TTTTTACGTCCACCCAGAAGCTTACCTGTCCCGGCTT |
| | | | | TCCTACGCGGGATGCCTTATTGGTCATCCTGATTCTG |
| | | | | CCATTGACAATCTCATCTG |
| MG178 | 7161 | MG178- | nucleo- | CACGTAACATGTAACAGCCGTTTTGACTGGATTGAC |
| recombinases | | 7193 AttL | tide | GGGCCTTGGCCATGGGAAGGAGGAATGGAATAATG |
| attachment | | | | TGGGTTTATGAAAAAAGACTTCAATTTCCGGTAAAC |
| sites | | | | ATCAAGCGGCCCGACCCGAAGGCGGCACAGATAAT |
| | | | | CATCAGCCAGTATGGTGGACTATTCTTCCGTAAGGT |
| | | | | ATAATATCATCACAAAGCCTTTATTTAAAGCAATTA |
| | | | | CAGGCTTTTTACGTCCACCCAGAAGCTTACCTGTCCC |
| | | | | GGCTTTCCTACGCGGGATGCCTTATTGGTCATCCTGA |
| | | | | TTCTGCCATTGACAATCTCATCTG |
| MG178 | 7162 | MG178- | nucleo- | CGATAAACTTACCAAAGCTAATGCTATCCGGTCCAT |
| recombinases | | 7193 AttR | tide | TGTTGACCACTGCGTCTATGACAAAGCAAACGACAG |
| attachment | | | | TTTTGATATATTTTTCTTTTTTTCTAAAGGCTCCGAAC |
| sites | | | | CCCTATAAAATAAGGCCTTATCATAATTATACCTTAC |
| | | | | GGAAGTATGGTGGACCGGACGGTGAAATGGGTGCC |
| | | | | AGCATGCGTTACCTCTCACAGCGCTATTCAATGCCTG |
| | | | | ACAATAAAGTTGCCGGACTGCTTACGGATATCGGCA |
| | | | | CCGAAGAGCTGGCGCATCTTGAGATAGTTGCCACAA |
| | | | | TGGTTCACCAGCTTACCAGAA |
| MG178 | 7163 | MG178- | nucleo- | TTGCGCAGAAAGGTACACCTCCTGCAGCCTGCGGCC |
| recombinases | | 7177 AttB | tide | ATACTCCAACCGTATGATCAACATAATATGCCTCAA |
| attachment | | | | ACGGAGTTCCTTTTATCTGTTCCATTGTAAGATTTCT |
| sites | | | | TGTAAGCTGATGAACCATTGTACTGACCATTTCAAG |
| | | | | ATGGCCAAGTTCTTCCGTGCCTATATCAGTCAGCAAT |
| | | | | CCGGCAACCTCCCTGTATGGCATGGAATATCTCTGT |
| | | | | GAAAGGTATCTCATTGATGCTCCAAGCTCACCATCT |
| | | | | GGTCCACCATACTGCGAAATAATCATTGCTGCCAGC |
| | | | | GTAGGATTACAATTCTT |
| MG178 | 7164 | MG178- | nucleo- | TGCTCCGTGTAAAAACATGCTGTTAAAATCTTGCATT |
| recombinases | | 7177 AttP | tide | GATAAAATTGTATATCACAATGACAGTGAATCAAAA |
| attachment | | | | GCTGGTATAGGCAGGTACGTTGATAATCCGTTCAAG |
| sites | | | | CTTGATATTTTCTTGCGCCTTTGATTTCCAACATCAA |
| | | | | GGTGCAAGTTCATTAGCTCATGGTTGTTGGATATGA |
| | | | | ATTTAACTTTAATATGTAAAGAATCTCTTCTTATTTA |
| | | | | TATATAGTTTAATATTAACCATGAAGGGGTAGTTTA |
| | | | | ACCTATTCCTTTATGGTTAATTTATATGTTCTTCCTGA |
| | | | | TTTTTCTCTAATAA |
| MG178 | 7165 | MG178- | nucleo- | TTGCGCAGAAAGGTACACCTCCTGCAGCCTGCGGCC |
| recombinases | | 7177 AttL | tide | ATACTCCAACCGTATGATCAACATAATATGCCTCAA |
| attachment | | | | ACGGAGTTCCTTTTATCTGTTCCATTGTAAGATTTCT |
| sites | | | | TGTAAGCTGATGAACCATTGTACTGACCATTTCAAG |
| | | | | ATGGCCAAGTTCATTAGCTCATGGTTGTTGGATATG |
| | | | | AATTTAACTTTAATATGTAAAGAATCTCTTCTTATTT |
| | | | | ATATATAGTTTAATATTAACCATGAAGGGGTAGTTT |
| | | | | AACCTATTCCTTTATGGTTAATTTATATGTTCTTCCTG |
| | | | | ATTTTTCTCTAATAA |
| MG178 | 7166 | MG178- | nucleo- | TGCTCCGTGTAAAAACATGCTGTTAAAATCTTGCATT |
| recombinases | | 7177 AttR | tide | GATAAAATTGTATATCACAATGACAGTGAATCAAAA |
| attachment | | | | GCTGGTATAGGCAGGTACGTTGATAATCCGTTCAAG |
| sites | | | | CTTGATATTTTCTTGCGCCTTTGATTTCCAACATCAA |
| | | | | GGTGCAAGTTCTTCCGTGCCTATATCAGTCAGCAATC |
| | | | | CGGCAACCTCCCTGTATGGCATGGAATATCTCTGTG |
| | | | | AAAGGTATCTCATTGATGCTCCAAGCTCACCATCTG |
| | | | | GTCCACCATACTGCGAAATAATCATTGCTGCCAGCG |
| | | | | TAGGATTACAATTCTT |
| MG178 | 7167 | MG178- | nucleo- | AACTCGCCGATTTTGCCGAGAACCTTGTCTTCCGCCG |
| recombinases | | 1859 AttB | tide | TTTCACGGACACAGCATGTATTCATCAAAATAACAT |
| attachment | | | | CGGCCAGTTCCGCATCCTCCGTCATCGTATAGCCGA |
| sites | | | | GCTCTTCAAGCTGTCCGGCATAACGCTCCGAATCGG |
| | | | | ACGAATTCATTTGACATCCGTATGTAATGAGACAAT |
| | | | | ACTGTTTATTGGTCAGCAAATCCATTTCTATATACAC |
| | | | | TCCGTTTATTACAACATAAAGTTCGGTTTTACATTTT |
| | | | | ATAATACCATATACGGCGTATATTCACCAGCACAGA |
| | | | | CAACAACTCTCAAAATTAGATGC |
| MG178 | 7168 | MG178- | nucleo- | TTGCCGGCAGACGGGTACAATCCATTACATTCGCCA |
| recombinases | | 1859 AttP | tide | ACGGCGTCACTCATACATTCACATATTAATTATATCC |
| attachment | | | | CGTCATAAAAGGCGGGATATTTTTTTGTAAAAAAAT |
| sites | | | | AAACGGCGCCATTGCGCCGTTCTTTTCATCTGATTTA |
| | | | | ATTGTTCATTTGACATCCATATGTAATTATAAATGCG |
| | | | | AATTTCTGTTTATGCATAGATATCCTCTCGTTGGTAT |
| | | | | TTTATACTATATAATATCATATCAACCACATAGAAA |
| | | | | ACAGGCTGGATAACCAACATCTTTATATCCAGCCTG |
| | | | | TTCTTTTTTAATAAGTTGTTTA |
| MG178 | 7169 | MG178- | nucleo- | AACTCGCCGATTTTGCCGAGAACCTTGTCTTCCGCCG |
| recombinases | | 1859 AttL | tide | TTTCACGGACACAGCATGTATTCATCAAAATAACAT |
| attachment | | | | CGGCCAGTTCCGCATCCTCCGTCATCGTATAGCCGA |
| sites | | | | GCTCTTCAAGCTGTCCGGCATAACGCTCCGAATCGG |
| | | | | ACGAATTCATTTGACATCCATATGTAATTATAAATGC |
| | | | | GAATTTCTGTTTATGCATAGATATCCTCTCGTTGGTA |
| | | | | TTTTATACTATATAATATCATATCAACCACATAGAAA |
| | | | | ACAGGCTGGATAACCAACATCTTTATATCCAGCCTG |
| | | | | TTCTTTTTTAATAAGTTGTTTA |
| MG178 | 7170 | MG178- | nucleo- | TTGCCGGCAGACGGGTACAATCCATTACATTCGCCA |
| recombinases | | 1859 AttR | tide | ACGGCGTCACTCATACATTCACATATTAATTATATCC |
| attachment | | | | CGTCATAAAAGGCGGGATATTTTTTTGTAAAAAAAT |
| sites | | | | AAACGGCGCCATTGCGCCGTTCTTTTCATCTGATTTA |
| | | | | ATTGTTCATTTGACATCCGTATGTAATGAGACAATAC |
| | | | | TGTTTATTGGTCAGCAAATCCATTTCTATATACACTC |
| | | | | CGTTTATTACAACATAAAGTTCGGTTTTACATTTTAT |
| | | | | AATACCATATACGGCGTATATTCACCAGCACAGACA |
| | | | | ACAACTCTCAAAATTAGATGC |
| MG178 | 7171 | MG178- | nucleo- | TCGGGTCAGCTGATAGAGGAGCGCCGAGATCATCTC |
| recombinases | | 7201 AttB | tide | GACATGCGCAAGCTCTTCAGTCCCGACGTCTGTAAG |
| attachment | | | | GATCCCTATCAGCTCCCCGTAGGGCATCGAATAGCG |
| sites | | | | CTGCTGAAGATAGCGGGTAGCCGCTCCGAGCTCGCC |
| | | | | ATGCGGACCGCCGAGCTGGCTAATAATGATCGAAGC |
| | | | | GCTCATCGGGTCGGGCTTTTGGACGCTGACCGGGTG |
| | | | | GATCAGCCGTTTATCATAGTTCCACATTTTCTTCCCT |
| | | | | CCTTACGCTTCGCTCTGCCACGGCCAGGGCGTTCGG |
| | | | | ACGTAATCCCAACAATC |
| MG178 | 7172 | MG178- | nucleo- | GCTTCTTCAGGATGAAAAAGCCTCTGACGCCGAAAA |
| recombinases | | 7201 AttP | tide | GAACGAGGCTTTGCACCATATCGTTGATAAAATCGT |
| attachment | | | | CTTTCAACGAAGCACCAATCACTTTGATGTTTTTTAC |
| sites | | | | TCGGAAAGCTATATGCTTTAGTATAACTTAATGTGAT |
| | | | | TAGCACCGCCAAATATCTTTAAGTTATACTACTGAC |
| | | | | AAACAGCATTTTTCTCAAAATGCCCCTATCACCAAG |
| | | | | CAGGCACTATATGAATTCCTTTCTTGCCATAATGGAT |
| | | | | TTTTATCCGGTTTGTCTTTTTAGACCCTCCTTTTAGAT |
| | | | | TTTTCGCCTCGCC |
| MG178 | 7173 | MG178- | nucleo- | TCGGGTCAGCTGATAGAGGAGCGCCGAGATCATCTC |
| recombinases | | 7201 AttL | tide | GACATGCGCAAGCTCTTCAGTCCCGACGTCTGTAAG |
| attachment | | | | GATCCCTATCAGCTCCCCGTAGGGCATCGAATAGCG |
| sites | | | | CTGCTGAAGATAGCGGGTAGCCGCTCCGAGCTCGCC |
| | | | | ATGCGGACCGCCAAATATCTTTAAGTTATACTACTG |
| | | | | ACAAACAGCATTTTTCTCAAAATGCCCCTATCACCA |
| | | | | AGCAGGCACTATATGAATTCCTTTCTTGCCATAATGG |
| | | | | ATTTTTATCCGGTTTGTCTTTTTAGACCCTCCTTTTAG |
| | | | | ATTTTTCGCCTCGCC |
| MG178 | 7174 | MG178- | nucleo- | GCTTCTTCAGGATGAAAAAGCCTCTGACGCCGAAAA |
| recombinases | | 7201 AttR | tide | GAACGAGGCTTTGCACCATATCGTTGATAAAATCGT |
| attachment | | | | CTTTCAACGAAGCACCAATCACTTTGATGTTTTTTAC |
| sites | | | | TCGGAAAGCTATATGCTTTAGTATAACTTAATGTGAT |
| | | | | TAGCACCGCCGAGCTGGCTAATAATGATCGAAGCGC |
| | | | | TCATCGGGTCGGGCTTTTGGACGCTGACCGGGTGGA |
| | | | | TCAGCCGTTTATCATAGTTCCACATTTTCTTCCCTCCT |
| | | | | TACGCTTCGCTCTGCCACGGCCAGGGCGTTCGGACG |
| | | | | TAATCCCAACAATC |
| MG178 | 7175 | MG178- | nucleo- | CCGCTTTTGTCACCCCCTGCTCGTAGGGAAGCGGCC |
| recombinases | | 7173 AttB | tide | AGTCATAGCCGTAGTTGGGGATTCCCAGGCTGATCT |
| attachment | | | | TTTCCGCCGGTATTTCCGTCAGGGCGTACTCTGCCAC |
| sites | | | | GCGTCTGACCATATTGATCGGGGCCACTGCCATGGG |
| | | | | CGGGCCATATGTGTATCCCCATTCATACGTCATCAGC |
| | | | | AGAACCCGGTTGGCCGCCTCTCCGAGCAGGCGGTAA |
| | | | | TCGATTCCTTCGTAAAGCAGCCCCCTCTGGTCTCTGG |
| | | | | CCGTCTTAGGAGCCAGTGCAACGCTCACCTGATAGC |
| | | | | CGAACAGGTTCATCAC |
| MG178 | 7176 | MG178- | nucleo- | TGGAGAAAGTTGATTATCTAAAAACCGAGAGAAAC |
| recombinases | | 7173 AttP | tide | AAAAAAGGCGGAAAGGGAAATGCTAATTTCTCTATT |
| attachment | | | | TATTTTTATCCACGCGTCCCGAAATACTGAAATTATG |
| sites | | | | TATACTTTTTCGGGGCGCATAATTACCTATGATTTAT |
| | | | | CGGCGCATATGTATAAGCGCCAACAATCATATGGTA |
| | | | | TCAAATTATAAAGGAAAGAAGGGGGAAAAATGGAC |
| | | | | AGAACAGACGAATTATTTTTTGAGATGATTGAAGCC |
| | | | | TATCAGCGGCACGCCCAGACTGCCAGACACGCAAAG |
| | | | | TTCAAGGAGACTCGCGAGA |
| MG178 | 7177 | MG178- | nucleo- | CCGCTTTTGTCACCCCCTGCTCGTAGGGAAGCGGCC |
| recombinases | | 7173 AttL | tide | AGTCATAGCCGTAGTTGGGGATTCCCAGGCTGATCT |
| attachment | | | | TTTCCGCCGGTATTTCCGTCAGGGCGTACTCTGCCAC |
| sites | | | | GCGTCTGACCATATTGATCGGGGCCACTGCCATGGG |
| | | | | CGGGCCATATGTATAAGCGCCAACAATCATATGGTA |
| | | | | TCAAATTATAAAGGAAAGAAGGGGGAAAAATGGAC |
| | | | | AGAACAGACGAATTATTTTTTGAGATGATTGAAGCC |
| | | | | TATCAGCGGCACGCCCAGACTGCCAGACACGCAAAG |
| | | | | TTCAAGGAGACTCGCGAGA |
| MG178 | 7178 | MG178- | nucleo- | TGGAGAAAGTTGATTATCTAAAAACCGAGAGAAAC |
| recombinases | | 7173 AttR | tide | AAAAAAGGCGGAAAGGGAAATGCTAATTTCTCTATT |
| attachment | | | | TATTTTTATCCACGCGTCCCGAAATACTGAAATTATG |
| sites | | | | TATACTTTTTCGGGGCGCATAATTACCTATGATTTAT |
| | | | | CGGCGCATATGTGTATCCCCATTCATACGTCATCAGC |
| | | | | AGAACCCGGTTGGCCGCCTCTCCGAGCAGGCGGTAA |
| | | | | TCGATTCCTTCGTAAAGCAGCCCCCTCTGGTCTCTGG |
| | | | | CCGTCTTAGGAGCCAGTGCAACGCTCACCTGATAGC |
| | | | | CGAACAGGTTCATCAC |
| MG178 | 7179 | MG178- | nucleo- | AAAACTCTCTATTGGTTTTTCCTTAGGTCGGCGACGA |
| recombinases | | 7198 AttB | tide | CCAACTGATAGCCCATGACACCTCAGTCAATCTAAT |
| attachment | | | | TTGTTCTCTCTGCTGTTATTCGGAACAACGATGAAAA |
| sites | | | | CACGCAAAAAAGACTTATCGTCTGTTGACGATAAGT |
| | | | | TCTTAACTGGTTGCGGGAGCTGGATTTGAACCAACG |
| | | | | ACCTTCGGGTTATGAGCCCGACGAGCTACCAAACTG |
| | | | | CTCCATCCCGCGATATTTATTTTTCAGTGCCTAATTA |
| | | | | TAATACCATATTTGCTTTACAAATGCAAGCCCTTTTT |
| | | | | TCAAAAAAATATAACAAATAATTAGAGGAA |
| MG178 | 7180 | MG178- | nucleo- | AGATTTTACAGTAGAGCAGATTCATTCATGGCTGGA |
| recombinases | | 7198 AttP | tide | AGCGCTGAAAGCCGCCCCGGATGATAAAGCTGTTCG |
| attachment | | | | CCTTTTGGTTTCTCGTATTGAGGTAAAACAAAAGAC |
| sites | | | | CGTCTTCAACATAGCAAGTACATTGAAAGCGGTCTT |
| | | | | ATGTGAAACTGGTTGCGGGAGCTGGATTGATATATT |
| | | | | ACCAAGAATCCTAATGAATTACCCCCAGCAGAACGA |
| | | | | GATTTAACAACCAGAATCAGACATATTTTCTGGAAG |
| | | | | TTCGACCCCTCCTTCCCCAATAAATTCTTCTATTGCA |
| | | | | TCATAAAGAGCTATAGACAAAGGGTCAGCGGCG |
| MG178 | 7181 | MG178- | nucleo- | AAAACTCTCTATTGGTTTTTCCTTAGGTCGGCGACGA |
| recombinases | | 7198 AttL | tide | CCAACTGATAGCCCATGACACCTCAGTCAATCTAAT |
| attachment | | | | TTGTTCTCTCTGCTGTTATTCGGAACAACGATGAAAA |
| sites | | | | CACGCAAAAAAGACTTATCGTCTGTTGACGATAAGT |
| | | | | TCTTAACTGGTTGCGGGAGCTGGATTGATATATTACC |
| | | | | AAGAATCCTAATGAATTACCCCCAGCAGAACGAGAT |
| | | | | TTAACAACCAGAATCAGACATATTTTCTGGAAGTTC |
| | | | | GACCCCTCCTTCCCCAATAAATTCTTCTATTGCATCA |
| | | | | TAAAGAGCTATAGACAAAGGGTCAGCGGCG |
| MG178 | 7182 | MG178- | nucleo- | AGATTTTACAGTAGAGCAGATTCATTCATGGCTGGA |
| recombinases | | 7198 AttR | tide | AGCGCTGAAAGCCGCCCCGGATGATAAAGCTGTTCG |
| attachment | | | | CCTTTTGGTTTCTCGTATTGAGGTAAAACAAAAGAC |
| sites | | | | CGTCTTCAACATAGCAAGTACATTGAAAGCGGTCTT |
| | | | | ATGTGAAACTGGTTGCGGGAGCTGGATTTGAACCAA |
| | | | | CGACCTTCGGGTTATGAGCCCGACGAGCTACCAAAC |
| | | | | TGCTCCATCCCGCGATATTTATTTTTCAGTGCCTAAT |
| | | | | TATAATACCATATTTGCTTTACAAATGCAAGCCCTTT |
| | | | | TTTCAAAAAAATATAACAAATAATTAGAGGAA |
| MG178 | 7183 | MG178- | nucleo- | ACCGGGCCTTTTTTTGTTCCTGCGATTTTAGTTTAGA |
| recombinases | | 7202 large | tide | AAATGTAGCCTATGGGCACCCCAAGGAAATAAACCC |
| attachment | | serine | | TACACTTGGAGCTTTCTTAGTTTGAGTAGTTGTCA |
| sites | | recombinase | | |
| | | attp_1 | | |
| MG178 | 7184 | MG178- | nucleo- | TTTTTGTTCCTGCGATTTTAGTTTAGAAAATGTAGCC |
| recombinases | | 7202 large | tide | TATGGGCACCCCAAGGAAATAAACCCTACACTTGGA |
| attachment | | serine | | GCTTTCTTAGTTTGA |
| sites | | recombinase | | |
| | | attp_2 | | |
| MG178 | 7185 | MG178- | nucleo- | TGCGATTTTAGTTTAGAAAATGTAGCCTATGGGCAC |
| recombinases | | 7202 large | tide | CCCAAGGAAATAAACCCTACACTTGGAGCTTT |
| attachment | | serine | | |
| sites | | recombinase | | |
| | | attp_3 | | |
| MG178 | 7186 | MG178- | nucleo- | TTTTAGTTTAGAAAATGTAGCCTATGGGCACCCCAA |
| recombinases | | 7202 large | tide | GGAAATAAACCCTACACTTGGA |
| attachment | | serine | | |
| sites | | recombinase | | |
| | | attp_4 | | |
| MG178 | 7187 | MG178- | nucleo- | GTTTAGAAAATGTAGCCTATGGGCACCCCAAGGAAA |
| recombinases | | 7202 large | tide | TAAACCCTACAC |
| attachment | | serine | | |
| sites | | recombinase | | |
| | | attp_5 | | |
| MG178 | 7188 | MG178- | nucleo- | CTTCCATGCGCAGCTGGGTCAGGATGCGTTCGGAGT |
| recombinases | | 7202 large | tide | CGACAGTTGCCTTCGGGCACCCAAGGCTGACGAATC |
| attachment | | serine | | CGACTTTCGGGGTGGCGGTGGACATGCGGGCTAACC |
| sites | | recombinase | | |
| | | attB_1 | | |
| MG178 | 7189 | MG178- | nucleo- | CAGCTGGGTCAGGATGCGTTCGGAGTCGACAGTTGC |
| recombinases | | 7202 large | tide | CTTCGGGCACCCAAGGCTGACGAATCCGACTTTCGG |
| attachment | | serine | | GGTGGCGGTGGACATG |
| sites | | recombinase | | |
| | | attB_2 | | |
| MG178 | 7190 | MG178- | nucleo- | AGGATGCGTTCGGAGTCGACAGTTGCCTTCGGGCAC |
| recombinases | | 7202 large | tide | CCAAGGCTGACGAATCCGACTTTCGGGGTGGC |
| attachment | | serine | | |
| sites | | recombinase | | |
| | | attB_3 | | |
| MG178 | 7191 | MG178- | nucleo- | GCGTTCGGAGTCGACAGTTGCCTTCGGGCACCCAAG |
| recombinases | | 7202 large | tide | GCTGACGAATCCGACTTTCGGG |
| attachment | | serine | | |
| sites | | recombinase | | |
| | | attB_4 | | |
| MG178 | 7192 | MG178- | nucleo- | CGGAGTCGACAGTTGCCTTCGGGCACCCAAGGCTGA |
| recombinases | | 7202 large | tide | CGAATCCGACTT |
| attachment | | serine | | |
| sites | | recombinase | | |
| | | attB_5 | | |
| MG178 | 7193 | MG178- | nucleo- | GGAGTCGACAGTTGCCTTCGGGCACCCAAGGCTGAC |
| recombinases | | 7202 large | tide | GAATCCGACT |
| attachment | | serine | | |
| sites | | recombinase | | |
| | | attB_6 | | |
| MG178 | 7194 | MG178- | nucleo- | GAGTCGACAGTTGCCTTCGGGCACCCAAGGCTGACG |
| recombinases | | 7202 large | tide | AATCCGAC |
| attachment | | serine | | |
| sites | | recombinase | | |
| | | attB_7 | | |
| MG178 | 7195 | MG178- | nucleo- | AGTCGACAGTTGCCTTCGGGCACCCAAGGCTGACGA |
| recombinases | | 7202 large | tide | ATCCGA |
| attachment | | serine | | |
| sites | | recombinase | | |
| | | attB_8 | | |
| MG178 | 7196 | MG178- | nucleo- | GTCGACAGTTGCCTTCGGGCACCCAAGGCTGACGAA |
| recombinases | | 7202 large | tide | TCCG |
| attachment | | serine | | |
| sites | | recombinase | | |
| | | attB_9 | | |
| MG178 | 7197 | MG178- | nucleo- | TCGACAGTTGCCTTCGGGCACCCAAGGCTGACGAAT |
| recombinases | | 7202 large | tide | CC |
| attachment | | serine | | |
| sites | | recombinase | | |
| | | attB_10 | | |
| MG178 | 7198 | MG178- | nucleo- | CGACAGTTGCCTTCGGGCACCCAAGGCTGACGAATC |
| recombinases | | 7202 large | tide | |
| attachment | | serine | | |
| sites | | recombinase | | |
| | | attB_11 | | |
| MG178 | 7199 | MG178- | nucleo- | ACAGTTGCCTTCGGGCACCCAAGGCTGACGAA |
| recombinases | | 7202 large | tide | |
| attachment | | serine | | |
| sites | | recombinase | | |
| | | attB_12 | | |
| MG178 | 7200 | MG178- | nucleo- | AGTTGCCTTCGGGCACCCAAGGCTGACG |
| recombinases | | 7202 large | tide | |
| attachment | | serine | | |
| sites | | recombinase | | |
| | | attB_13 | | |
| MG178 | 7201 | MG178- | nucleo- | GGCTCCGAACCCCTATAAAATAAGGCCTTATCATAA |
| recombinases | | 7193 large | tide | TTATACCTTACGGAAGTATGGTGGACTATTCTTCCGT |
| attachment | | serine | | AAGGTATAATATCATCACAAAGCCTTTATTTAAAGC |
| sites | | recombinase | | AAT |
| | | attp_1 | | |
| MG178 | 7202 | MG178- | nucleo- | CCCTATAAAATAAGGCCTTATCATAATTATACCTTAC |
| recombinases | | 7193 large | tide | GGAAGTATGGTGGACTATTCTTCCGTAAGGTATAAT |
| attachment | | serine | | ATCATCACAAAGCCTTTAT |
| sites | | recombinase | | |
| | | attp_2 | | |
| MG178 | 7203 | MG178- | nucleo- | TAAGGCCTTATCATAATTATACCTTACGGAAGTATG |
| recombinases | | 7193 large | tide | GTGGACTATTCTTCCGTAAGGTATAATATCATCACA |
| attachment | | serine | | |
| sites | | recombinase | | |
| | | attp_3 | | |
| MG178 | 7204 | MG178- | nucleo- | CCTTATCATAATTATACCTTACGGAAGTATGGTGGA |
| recombinases | | 7193 large | tide | CTATTCTTCCGTAAGGTATAATATCA |
| attachment | | serine | | |
| sites | | recombinase | | |
| | | attp_4 | | |
| MG178 | 7205 | MG178- | nucleo- | TCATAATTATACCTTACGGAAGTATGGTGGACTATTC |
| recombinases | | 7193 large | tide | TTCCGTAAGGTATAA |
| attachment | | serine | | |
| sites | | recombinase | | |
| | | attp_5 | | |
| MG178 | 7206 | MG178- | nucleo- | GGTAAACATCAAGCGGCCCGACCCGAAGGCGGCAC |
| recombinases | | 7193 large | tide | AGATAATCATCAGCCAGTATGGTGGACCGGACGGTG |
| attachment | | serine | | AAATGGGTGCCAGCATGCGTTACCTCTCACAGCGCT |
| sites | | recombinase | | ATTCA |
| | | attB_1 | | |
| MG178 | 7207 | MG178- | nucleo- | AAGCGGCCCGACCCGAAGGCGGCACAGATAATCAT |
| recombinases | | 7193 large | tide | CAGCCAGTATGGTGGACCGGACGGTGAAATGGGTGC |
| attachment | | serine | | CAGCATGCGTTACCTCTCACA |
| sites | | recombinase | | |
| | | attB_2 | | |
| MG178 | 7208 | MG178- | nucleo- | ACCCGAAGGCGGCACAGATAATCATCAGCCAGTATG |
| recombinases | | 7193 large | tide | GTGGACCGGACGGTGAAATGGGTGCCAGCATGCGTT |
| attachment | | serine | | |
| sites | | recombinase | | |
| | | attB_3 | | |
| MG178 | 7209 | MG178- | nucleo- | AAGGCGGCACAGATAATCATCAGCCAGTATGGTGGA |
| recombinases | | 7193 large | tide | CCGGACGGTGAAATGGGTGCCAGCAT |
| attachment | | serine | | |
| sites | | recombinase | | |
| | | attB_4 | | |
| MG178 | 7210 | MG178- | nucleo- | GGCACAGATAATCATCAGCCAGTATGGTGGACCGGA |
| recombinases | | 7193 large | tide | CGGTGAAATGGGTGCC |
| attachment | | serine | | |
| sites | | recombinase | | |
| | | attB_5 | | |
| MG178 | 7211 | MG178- | protein | MAKRELMKNLMSDTFRRVAIYIRVSTNHQVDKDSLPL |
| recombinases | | 7205 large | | QREELINYCKYVLGIEDFEIFEDAGFSAKNTDRPGYQK |
| | | serine | | MMKMVRAGLFTHVLVWKLDRISRNLLDFAYMYEELK |
| | | recombinase | | KLDVAFISKNEQFDTSTAMGEAMLKIILVFAELERKTTS |
| | | | | ERVAATMISRAVNGKWNGGRVPYGYSYDYEQKEFSV |
| | | | | NPEEQKVALLMCDLYEASNSLLFVSRKLNEIGYRSRAG |
| | | | | NLWSPVQVRKVLVNPFNTGKYVYNQTSLSTGTQLPNK |
| | | | | EEDFIVIEDHHPALIPQERQDRLIARLNRNARSRSTANN |
| | | | | TTNRKNIHVFSGLIYCDNCGNMLTSSVGKKLAGDGWR |
| | | | | PSIYLCPSKRKHVSDGCHDTTDSSVGEFVLNFIMNMLN |
| | | | | AQRNFQLVRSAADLQRLLLYGKVFDDVDHLDQDSLN |
| | | | | DMFTVLSSNLPQNVKAGMKKKMRKPPSNPEVTKLNK |
| | | | | EKQRLERAMERLKRLYMYSDDSMTEQEYITEKNRIAD |
| | | | | AYSEAEARIVEITNYERMERSISDEDFVRQATAFILSKK |
| | | | | LSGSSYINYRKLAVSTDPLMLKEFFNSILDSITINTDGKV |
| | | | | GSIVFKNGLRHQFIYTSNKKEDKTMLAKCNYCGQVML |
| | | | | EADGCTKTYFTLNGKQYPRIRVGDKYDFEPGTTSRCH |
| | | | | DCAAKPGEYHHSGCDAERCPVCHEQLIGCECDFSDL |
| MG178 | 7212 | MG178- | protein | MPRAQKAAIYCRVSTLHQVDKDSLPMQRQDMINYAK |
| recombinases | | 7206 large | | YALGIEDYEVFEDAGYSGKNTDRPAFQDMMERIEAGE |
| | | serine | | FSHVLVWKIDRISRNLLDFATMYAKCKKLGVTFVSKN |
| | | recombinase | | EQFDTSTAMGEAMLKIILVFAELERNMTSERVSSTMNA |
| | | | | RAAEGKWNGGRVPFAYRYDRETDSFSIRDDEAKVALE |
| | | | | LKDIYLRTRSLTYTARTLNEAGKRTRRGYAWTPATVAI |
| | | | | ILRSPFYRGTYRYNYRDESETTFSFKGQNDWIMCAKHH |
| | | | | PPLFSEADCRQIDFWLTKNRRQHGKATHVQRKLVHVF |
| | | | | AGLIRCGFCGSNYIASIDRVRASGYQPSMYNCGGRRQK |
| | | | | GMCKNRYVTDIRIGGFVFNYISNVIRLRDAFRPQWSKA |
| | | | | RIQRLLLRGDDFQCVSLSDDTLRRVRAALLGSRLSTAE |
| | | | | YQPADEQNDDSAEQTRKSNLQAELEKNRRALSRLMHL |
| | | | | FLYAEDEMPQADFLREKKHLQDTITRLQAELEKAQQSS |
| | | | | VFASSLTDEEFLGKAAHLLFQSAMVRGGIDFPELAMRV |
| | | | | GNLELKNFVNAVIKQIIVLDGRVTQITFANDEIHTFLYH |
| MG178 | 7213 | MG178- | protein | MEEIKCAIYTRKSTDEGLEKEFNTLEAQREAGENYVKS |
| recombinases | | 7207 large | | QKHQGWILVDEHYDDGGFSGGNMKRPALQRLFKDIEL |
| | | serine | | GKINMIVVYKIDRLTRSLVDFSKMVDIFDKYHCSFVSV |
| | | recombinase | | TQNFNTSDSMGRLTLNMLLSFAQFEREIGSERVRDKTA |
| | | | | ASRKKGMWTGGTVPFGYRSVNKKLEIEPNEAEAVKF |
| | | | | MFEMYIKYKSAMAVCKLLTEKGYRAFRRDAVLRMLK |
| | | | | NPIYEGKIKYKHELYDGQHQAIIRQKTFEAVQYILLNK |
| | | | | DKRERTCLFNRNEVGILRGLLICGCCHAPMTPASCQSH |
| | | | | GVRRYYYTSTKAKYYGYHHCSNGAVPVALMDECMT |
| | | | | KIVTPLISDINVLNGLINKICPDKSAEIYKVMRNPEKIIER |
| | | | | MTERDKLQLMKLLIKKIIVNYDTIEINWSDLALSLLPAY |
| | | | | LRARTQNQITIIDYPFKRNKGALTLSLPEEVAPNINYNA |
| | | | | ELITALCKAFKYQKIMNKEKQSIIELAANENIDSGYLGR |
| | | | | LIRLTCLAPDIIKRILEGTQPTTIYLKRLLREDIPPIWQDQ |
| | | | | RIKYGFVK |
| MG178 | 7214 | MG178- | protein | MIAIYARQSIEKKDSVSIEAQIEKCKYYCENQDYKIYKD |
| recombinases | | 7208 large | | AGYSGKNINRPQFSKLLEDIKSGLITKVIAYRLDRISRSI |
| | | serine | | ADFSQLLILFDEHNVDFVSATENFDTNTPMGRAMINIV |
| | | recombinase | | MTFAQLERETIVERVTDNYYFRANNGYWAGGYAPYG |
| | | | | YEIKHIIGNDGKRHSVLVENKDESKIVKEIYDMYINQNI |
| | | | | SMRKIAQQLNYQNIPTKKQSGNWGINAVNAILSRPIYT |
| | | | | EATTKIYDYFNKRGTCITNNIEHFDGTKTANLYGNSKK |
| | | | | NNNVKALRNYDEMFLSLINCVPLISNEDWFKVQNIKGT |
| | | | | KKNLPPRTNSSKISFLCGLVKCGKCSSNMVTQGCKNR |
| | | | | YGIQYYYLICSTKRNLGRIKCDNKMIDISKLEDIVINDIK |
| | | | | KHFNSNEIETKIEKYMKNNKKENINLLKQKEEFENKIIK |
| | | | | IDIQIQNLINSIAEGNITISKYINQKIEVLEKEKQNISSQLS |
| | | | | TIIEKNNITQDNYLIEYVKNINEKINTKDFEQLKLLCHTII |
| | | | | DKIVITDKNIDIHYKI |
| MG178 | 7215 | MG178- | nucleo- | CGCTTCAAGGCGACGGAGGGACTGATTACCAACTTC |
| recombinases | | 7163 AttB | tide | CATTTACCGCAATCGACGCTGCTTATGCTTGTTTCGG |
| attachment | | | | CGTTCAGCTCACGGGAGATAATGATGAACGCATATG |
| sites | | | | AGACGGCGAAGAGAGAACAATATCGATTTTTCAGTT |
| | | | | TCGGAGATGCGATGTTTATGCGATAGCATCAAAAAA |
| | | | | CGGGCGCTTTTTATAAGCTGCCCGTTTTTCTGTTATT |
| | | | | TGTTATTTGAATTATTTCAGCTGAAAGCCTGTTCAAT |
| | | | | TCCCCAGTCGCAGAGGAATAGGATGGGATAGGTATA |
| | | | | GCAAAGGTGAAAGAGGCTGGTGTTCCATTTTACG |
| MG178 | 7216 | MG178- | nucleo- | CGCTTCAAGGCGACGGAGGGACTGATTACCAACTTC |
| recombinases | | 7163 AttL | tide | CATTTACCGCAATCGACGCTGCTTATGCTTGTTTCGG |
| attachment | | | | CGTTCAGCTCACGGGAGATAATGATGAACGCATATG |
| sites | | | | AGACGGCGAAGAGAGAACAATATCGATTTTTCAGTT |
| | | | | TCGGAGATGCGATGTTTATGCGATAGCATCATTTGTT |
| | | | | ACGGAATAATAACGACAAGAAAAGCCCAGAACACG |
| | | | | CTTGGTTTCTGGGCTTTTTGTTGTTCAATCGTCTTTGT |
| | | | | ACATAAATTCGTGGATTATGCCGTTTTTGAAGGTAAT |
| | | | | TGACCGGATTTTACCGTTTTCTATACAAAAGTT |
| MG178 | 7217 | MG178- | nucleo- | GAAAGACGAGGAAACGTCCGATAAATAAAAAAACG |
| recombinases | | 7163 AttP | tide | CCTTAAAAGGCGTCACAGGCGGTTTGTGAGGAAGCA |
| attachment | | | | AGTCAACAGAGATAAGGACTTGCGTAATTTATAAGC |
| sites | | | | GCCATTTCTGGGCGAGTAATGATGATATCCCGTGAG |
| | | | | CATCGGGGATGCGATGTTTATGCGATAGCATCATTT |
| | | | | GTTACGGAATAATAACGACAAGAAAAGCCCAGAAC |
| | | | | ACGCTTGGTTTCTGGGCTTTTTGTTGTTCAATCGTCTT |
| | | | | TGTACATAAATTCGTGGATTATGCCGTTTTTGAAGGT |
| | | | | AATTGACCGGATTTTACCGTTTTCTATACAAAAGTT |
| MG178 | 7218 | MG178- | nucleo- | GAAAGACGAGGAAACGTCCGATAAATAAAAAAACG |
| recombinases | | 7163 AttR | tide | CCTTAAAAGGCGTCACAGGCGGTTTGTGAGGAAGCA |
| attachment | | | | AGTCAACAGAGATAAGGACTTGCGTAATTTATAAGC |
| sites | | | | GCCATTTCTGGGCGAGTAATGATGATATCCCGTGAG |
| | | | | CATCGGGGATGCGATGTTTATGCGATAGCATCAAAA |
| | | | | AACGGGCGCTTTTTATAAGCTGCCCGTTTTTCTGTTA |
| | | | | TTTGTTATTTGAATTATTTCAGCTGAAAGCCTGTTCA |
| | | | | ATTCCCCAGTCGCAGAGGAATAGGATGGGATAGGTA |
| | | | | TAGCAAAGGTGAAAGAGGCTGGTGTTCCATTTTACG |
| MG178 | 7219 | MG178- | nucleo- | GATGCGATGTTTATGCGATAGCATC |
| conserved | | 7163 Core | tide | |
| core | | | | |
| MG178 | 7220 | MG178- | nucleo- | TACCGTTTTAAGGTATTGGATGCCCTGATTACGAATT |
| recombinases | | 7205 AttB | tide | TCCACCTGCCCCAGTCCACACTGGTGATGCTGGTCA |
| attachment | | | | GCGCGCTGGCCGGACGGGAGCATATTTTAAACGCCT |
| sites | | | | ACCGGGAAGCAGTGAAGGAACGTTACCGTTTCTTCT |
| | | | | CCTTTGGGGATGCTATGTTTATCGCGGCCCATCCGGC |
| | | | | TGCTGAGAAGCGGCAGGGATTGTGGGAATGACAGA |
| | | | | AGAAAGCGGGAGAAAATGGAAGAACGTTTAAATAA |
| | | | | ATGGCTGAGCCGGATGGGAGTCTGCTCCAGACGCGA |
| | | | | GGCGGACCGTCTGATTGAGGCCGG |
| MG178 | 7221 | MG178- | nucleo- | TACCGTTTTAAGGTATTGGATGCCCTGATTACGAATT |
| recombinases | | 7205 AttL | tide | TCCACCTGCCCCAGTCCACACTGGTGATGCTGGTCA |
| attachment | | | | GCGCGCTGGCCGGACGGGAGCATATTTTAAACGCCT |
| sites | | | | ACCGGGAAGCAGTGAAGGAACGTTACCGTTTCTTCT |
| | | | | CCTTTGGGGATGCTATGCTTATACTATAACACCAAA |
| | | | | AATAAACTGTACAGTTGACACTAAAAAAGCAGCCAG |
| | | | | GAGGTCAATTCTCCTGGCCGTTTTGATTATAAGTCTG |
| | | | | AAAAATCGCACTCACAGCCAATTAGCTGTTCATGGC |
| | | | | ATACGGGGCAACGCTCCGCATC |
| MG178 | 7222 | MG178- | nucleo- | GTTGGAGTTCCTGGGCTTTTCTGCATGTAGAAGAAG |
| recombinases | | 7205 AttP | tide | TTCGTTGCCTGCCGGATGATTTGGTGATATAATGCAG |
| attachment | | | | TGTATATGAAAAAAATGAAAGAAGTGGAAAGAATG |
| sites | | | | ATAAACCGTACAGTTAATTTTAGGTGCAGACCCGTG |
| | | | | AGTATGGGGGATGCTATGCTTATACTATAACACCAA |
| | | | | AAATAAACTGTACAGTTGACACTAAAAAAGCAGCCA |
| | | | | GGAGGTCAATTCTCCTGGCCGTTTTGATTATAAGTCT |
| | | | | GAAAAATCGCACTCACAGCCAATTAGCTGTTCATGG |
| | | | | CATACGGGGCAACGCTCCGCATC |
| MG178 | 7223 | MG178- | nucleo- | GTTGGAGTTCCTGGGCTTTTCTGCATGTAGAAGAAG |
| recombinases | | 7205 AttR | tide | TTCGTTGCCTGCCGGATGATTTGGTGATATAATGCAG |
| attachment | | | | TGTATATGAAAAAAATGAAAGAAGTGGAAAGAATG |
| sites | | | | ATAAACCGTACAGTTAATTTTAGGTGCAGACCCGTG |
| | | | | AGTATGGGGGATGCTATGTTTATCGCGGCCCATCCG |
| | | | | GCTGCTGAGAAGCGGCAGGGATTGTGGGAATGACA |
| | | | | GAAGAAAGCGGGAGAAAATGGAAGAACGTTTAAAT |
| | | | | AAATGGCTGAGCCGGATGGGAGTCTGCTCCAGACGC |
| | | | | GAGGCGGACCGTCTGATTGAGGCCGG |
| MG178 | 7224 | MG178- | nucleo- | GGGGATGCTATG |
| conserved | | 7205 Core | tide | |
| core | | | | |
| MG178 | 7225 | MG178- | nucleo- | TACCGCTTCAAGGTGCTGGATGCCCTGGTCACCAAT |
| recombinases | | 7165 AttB_1 | tide | TTCCACCTGCCCCAGTCCACCCTGATCATGCTGGTGT |
| attachment | | | | CCGCCCTGGCCGGGCGGGAGCACGTTCTGGCCGCCT |
| sites | | | | ATGAGGAGGCTGTGAAGGAGCGCTACCGCTTTTTCA |
| | | | | GCTTCGGAGATGCCATGTTCATCTCCTGATTCCAAAT |
| | | | | CATAAAATACGCAGAGATCCGACGGATCTCGTCAAA |
| | | | | GGAGTTTTTTTGTGTTTGAAGTGATCAAGACCGAGG |
| | | | | GGAACGCCCGCCGGGGCGTATTTACCTGCCCCCACG |
| | | | | GCACTGTCCAGACCCCTGTCTTTATGA |
| MG178 | 7226 | MG178- | nucleo- | TACCGCTTCAAGGTGCTGGATGCCCTGGTCACCAAT |
| recombinases | | 7165 AttB_2 | tide | TTCCACCTGCCCCAGTCCACCCTGATCATGCTGGTGT |
| attachment | | | | CCGCCCTGGCCGGGCGGGAGCACGTTCTGGCCGCCT |
| sites | | | | ATGAGGAGGCTGTGAAGGAGCGCTACCGCTTTTTCA |
| | | | | GCTTCGGAGATGCGATGTTCATCTCCTGATTCCAAAT |
| | | | | CATAAAATACGCAGAGATCCGACGGATCTCGTCAAA |
| | | | | GGAGTTTTTTTGTGTTTGAAGTGATCAAGACCGAGG |
| | | | | GGAACGCCCGCCGGGGCGTATTTACCTGCCCCCACG |
| | | | | GCACTGTCCAGACCCCTGTCTTTATGA |
| MG178 | 7227 | MG178- | nucleo- | TACCGCTTCAAGGTGCTGGATGCCCTGGTCACCAAT |
| recombinases | | 7165 AttL | tide | TTCCACCTGCCCCAGTCCACCCTGATCATGCTGGTGT |
| attachment | | | | CCGCCCTGGCCGGGCGGGAGCACGTTCTGGCCGCCT |
| sites | | | | ATGAGGAGGCTGTGAAGGAGCGCTACCGCTTTTTCA |
| | | | | GCTTCGGAGATGCGATGTTCATTGGAGACTAGATAC |
| | | | | AGAAATTTCCAGAGGACTGAGAAAAAGCCCAGGAA |
| | | | | CCAAGGGGTTCCTGGGCTTTTTGCTGTCATAGGTCGT |
| | | | | TGTAGAAAAATTGGAGCTCTATGCCGTTTTTGAAGC |
| | | | | GGATTGAGACGACTCGGCCGTTTTTTAT |
| MG178 | 7228 | MG178- | nucleo- | GGTAGCGACGATTGCCAGGACAGCTATGCCAAGTAT |
| recombinases | | 7165 AttP_1 | tide | CTCACCAACGTCTGCAAGTACGATGAAAAGTACGCA |
| attachment | | | | AAGAGCATGGCGGATCTGCAATTCCGAAGTTGCTCG |
| sites | | | | AAAAGTGTTACCAATGCGGAAATCTGGTCTCCCGTG |
| | | | | AGTATGGGAGATGCCATGTTCATTGGAGACTAGATA |
| | | | | CAGAAATTTCCAGAGGACTGAGAAAAAGCCCAGGA |
| | | | | ACCAAGGGGTTCCTGGGCTTTTTGCTGTCATAGGTCG |
| | | | | TTGTAGAAAAATTGGAGCTCTATGCCGTTTTTGAAG |
| | | | | CGGATTGAGACGACTCGGCCGTTTTTTAT |
| MG178 | 7229 | MG178- | nucleo- | GGTAGCGACGATTGCCAGGACAGCTATGCCAAGTAT |
| recombinases | | 7165 AttP_2 | tide | CTCACCAACGTCTGCAAGTACGATGAAAAGTACGCA |
| attachment | | | | AAGAGCATGGCGGATCTGCAATTCCGAAGTTGCTCG |
| sites | | | | AAAAGTGTTACCAATGCGGAAATCTGGTCTCCCGTG |
| | | | | AGTATGGGAGATGCGATGTTCATTGGAGACTAGATA |
| | | | | CAGAAATTTCCAGAGGACTGAGAAAAAGCCCAGGA |
| | | | | ACCAAGGGGTTCCTGGGCTTTTTGCTGTCATAGGTCG |
| | | | | TTGTAGAAAAATTGGAGCTCTATGCCGTTTTTGAAG |
| | | | | CGGATTGAGACGACTCGGCCGTTTTTTAT |
| MG178 | 7230 | MG178- | nucleo- | GGTAGCGACGATTGCCAGGACAGCTATGCCAAGTAT |
| recombinases | | 7165 AttR | tide | CTCACCAACGTCTGCAAGTACGATGAAAAGTACGCA |
| attachment | | | | AAGAGCATGGCGGATCTGCAATTCCGAAGTTGCTCG |
| sites | | | | AAAAGTGTTACCAATGCGGAAATCTGGTCTCCCGTG |
| | | | | AGTATGGGAGATGCCATGTTCATCTCCTGATTCCAA |
| | | | | ATCATAAAATACGCAGAGATCCGACGGATCTCGTCA |
| | | | | AAGGAGTTTTTTTGTGTTTGAAGTGATCAAGACCGA |
| | | | | GGGGAACGCCCGCCGGGGCGTATTTACCTGCCCCCA |
| | | | | CGGCACTGTCCAGACCCCTGTCTTTATGA |
| MG178 | 7231 | MG178- | nucleo- | GGAGATGCCATGTTCAT |
| conserved | | 7165 Core 1 | tide | |
| core | | | | |
| MG178 | 7232 | MG178- | nucleo- | GGAGATGCGATGTTCAT |
| conserved | | 7165 Core 2 | tide | |
| core | | | | |
| MG178 | 7233 | MG178- | nucleo- | AAGTCCACGTTGATCATGCTCATCAGCGCCTTCGCC |
| recombinases | | 7206 AttB | tide | GGCCGCAATTTCGTGCTGAACGCCTACAAGACCGCC |
| attachment | | | | GTCGAGATGAAGTACCGCTTTTTCTCGTTTGGCGATG |
| sites | | | | CAATGTTCTGCTCACGCAAGCAACCAGACGCCGAGC |
| | | | | GAGCCGAAGAACTCAAGGAGCTTGAGGAGCTCGAC |
| | | | | CGCCAGCGCGAGGCCGAGGGAAAAGCATAATCTAC |
| | | | | GTTATGATAGCGAAAGAGCCGCATGTCATCGGAACA |
| | | | | TGCGGCTCTTTTTTGTATGATGTTGATTTGTCTTATTT |
| | | | | CCGCTGCAGGCGCTTCCAGCCGAGGTAGAGGTGGCT |
| | | | | GGCGAAGAAGAAG |
| MG178 | 7234 | MG178- | nucleo- | AAGTCCACGTTGATCATGCTCATCAGCGCCTTCGCC |
| recombinases | | 7206 AttL | tide | GGCCGCAATTTCGTGCTGAACGCCTACAAGACCGCC |
| attachment | | | | GTCGAGATGAAGTACCGCTTTTTCTCGTTTGGCGATG |
| sites | | | | CAATGTTCTGCTCACGCAAGCAACCAGACGCCGAGC |
| | | | | GAGCCGAAGAACTCAAGGAGCTTGAGGAGCTCGAC |
| | | | | CGCCAGCGTGAGGCCGAGGCCGCGAAAAAAGAATG |
| | | | | ATGTGACAGTAAAAGCCTTCCCTGCGCGGGAAGGCT |
| | | | | TTTTTCAATGGTAGAGGAAAGTGTGGATTTCGTCATT |
| | | | | GGCGAAGGTGATCTGGGTGACGCGGCCATCGAGCAC |
| | | | | GATGATTTGCTTGA |
| MG178 | 7235 | MG178- | nucleo- | GCAGCGGGATCCTCAGCATGGTGCAGGGCAAGACC |
| recombinases | | 7206 AttP | tide | GGTAAGGCGGCAACGGAGTAATGCTATATAATAGTA |
| attachment | | | | GACAGTGGCTTGCTTAACTCAATGATACATTGGGGA |
| sites | | | | CGCCATGTTCATTCGCCGCAAGCAGCCCGATGCGGA |
| | | | | GCGCGCGGAAGAACTCAAGGAGCTTGAGGAGCTCG |
| | | | | ACCGCCAGCGTGAGGCCGAGGCCGCGAAAAAAGAA |
| | | | | TGATGTGACAGTAAAAGCCTTCCCTGCGCGGGAAGG |
| | | | | CTTTTTTCAATGGTAGAGGAAAGTGTGGATTTCGTCA |
| | | | | TTGGCGAAGGTGATCTGGGTGACGCGGCCATCGAGC |
| | | | | ACGATGATTTGCTTGA |
| MG178 | 7236 | MG178- | nucleo- | GCAGCGGGATCCTCAGCATGGTGCAGGGCAAGACC |
| recombinases | | 7206 AttR | tide | GGTAAGGCGGCAACGGAGTAATGCTATATAATAGTA |
| attachment | | | | GACAGTGGCTTGCTTAACTCAATGATACATTGGGGA |
| sites | | | | CGCCATGTTCATTCGCCGCAAGCAGCCCGATGCGGA |
| | | | | GCGCGCGGAAGAACTCAAGGAGCTTGAGGAGCTCG |
| | | | | ACCGCCAGCGCGAGGCCGAGGGAAAAGCATAATCT |
| | | | | ACGTTATGATAGCGAAAGAGCCGCATGTCATCGGAA |
| | | | | CATGCGGCTCTTTTTTGTATGATGTTGATTTGTCTTAT |
| | | | | TTCCGCTGCAGGCGCTTCCAGCCGAGGTAGAGGTGG |
| | | | | CTGGCGAAGAAGAAG |
| MG178 | 7237 | MG178- | nucleo- | GAAGAACTCAAGGAGCTTGAGGAGCTCGACCGCCA |
| conserved | | 7206 Core | tide | GCG |
| core | | | | |
| MG178 | 7238 | MG178- | nucleo- | TCCATTCGTATTATATGGCATAGGCTCTGCTTCGCCT |
| recombinases | | 7208 AttB | tide | CTATAATATGTTTCCATTCTATCTGTATCATTATTAA |
| attachment | | | | AACTAATATTTTTGCCAAGAAAAATCCCCCTCATAT |
| sites | | | | AATAATTTTATTTTATTATATGAAA |
| MG178 | 7239 | MG178- | nucleo- | TCTTTGGCTATTCCAACTTTGCATTGTCCTTTTTGTTG |
| recombinases | | 7208 AttL | tide | TCCATAATATGTTTGATTTGCTAGAGCCTCTAAATTC |
| attachment | | | | TCTAACTTTCAATGTTCCATTCGTATTATATGGCATA |
| sites | | | | GGCTCTGCTTCGCCTCTATAATATGTTTCCATTCTAT |
| | | | | CTGTATCATTATTAAAATCAAAATGGCATACTATTTG |
| | | | | TTGTAATAATTAAAACATTCAATCATGCCTTTTCTAT |
| | | | | ATACTTTTAAGAATGTTTTTGAAAACATTCTTACTAA |
| | | | | AATTTTTTCTTTAAAACTTAATCCATTTAATGCACTA |
| | | | | TTTAACACAATATCTTTTG |
| MG178 | 7240 | MG178- | nucleo- | ATAACCGATAAAAATATAGATATACACTACAAGATT |
| recombinases | | 7208 AttP | tide | TAGTGTATGTCTTTTTGAATTTAATGTATCATTATTA |
| attachment | | | | AAATCAAAATGGCATACTATTTGTTGTAATAATTAA |
| sites | | | | AACATTCAATCATGCCTTTTCTATATA |
| MG178 | 7241 | MG178- | nucleo- | GAATACGTTAAAAACATTAATGAAAAAATCAATACT |
| recombinases | | 7208 AttR | tide | AAAGACTTTGAACAATTAAAATTACTTTGTCACACA |
| attachment | | | | ATTATAGATAAAATAGTTATAACCGATAAAAATATA |
| sites | | | | GATATACACTACAAGATTTAGTGTATGTCTTTTTGAA |
| | | | | TTTAATGTATCATTATTAAAAACTAATATTTTTGCCA |
| | | | | AGAAAAATCCCCCTCATATAATAATTTTATTTTATTA |
| | | | | TATGAAAGGGAATATCTATTTGTGACTATATTTTTAC |
| | | | | CTTTATTTTACTTCTTTACATCTTACAATTACTCTTAA |
| | | | | TTTACTATCATCTGTACATGTA |
| MG178 | 7242 | MG178- | nucleo- | TGTATCATTATTAAAA |
| conserved | | 7208 Core | tide | |
| core | | | | |
| MG178 | 7243 | MG178- | nucleo- | CCTTATTAACCATTTGAAAAATATAAATATTGATAA |
| recombinases | | 7207 AttB | tide | AAAATAGTTCGGATATTTCAAAAAAATAATGTTTTT |
| attachment | | | | CAAAAAACGAACATCCGAACTCATCTAACCTATTGA |
| sites | | | | TTTATAAAAGAAAAACAGCTCCCGTGATAGGAGCTG |
| | | | | TTAACTATGGTGGGCCCTGCCTGACTCGAACAGGCG |
| | | | | ACCAGACCGTTATGAGCGGCCTGCTCTAACCAACTG |
| | | | | AGCTAAGGGCCCGGCATCTGAAAACGGTTAAAAAAT |
| | | | | AGGTCAAATATTTTCCGAAGTCAAGCGCTTTTTGGG |
| | | | | CACCCCGCCGATCCGCGCCGC |
| MG178 | 7244 | MG178- | nucleo- | CCTTATTAACCATTTGAAAAATATAAATATTGATAA |
| recombinases | | 7207 AttL | tide | AAAATAGTTCGGATATTTCAAAAAAATAATGTTTTT |
| attachment | | | | CAAAAAACGAACATCCGAACTCATCTAACCTATTGA |
| sites | | | | TTTATAAAAGAAAAACAGCTCCCGTGATAGGAGCTG |
| | | | | TTAACTATGGTGGGCGATACGCATCACACAGGCGAA |
| | | | | CGCACAAAAATGTATCTAATATTTTTCAAATTCAAG |
| | | | | GGGATATTTGTCATTACACAAGCCGCCAATATCCAT |
| | | | | TTTATGGTATGGCTTAAGCATTATAAAAAAAGGCCA |
| | | | | CCTTTTATTAAGGTGGTCGGA |
| MG178 | 7245 | MG178- | nucleo- | AGTATCTATTTTGCCCTGATATCAGAAAAAAACGCT |
| recombinases | | 7207 AttP | tide | AAAAAAAGGATATAAAAAAAGAGATAACGCTCAGA |
| attachment | | | | CCGGCTATCGCGAACACCGCGGAAATACTGCCGTTC |
| sites | | | | AGAGGATACAAAAAAAGCCTCTTAATTTAAGAGGCT |
| | | | | TTTGATGATGGTGGGCGATACGCATCACACAGGCGA |
| | | | | ACGCACAAAAATGTATCTAATATTTTTCAAATTCAA |
| | | | | GGGGATATTTGTCATTACACAAGCCGCCAATATCCA |
| | | | | TTTTATGGTATGGCTTAAGCATTATAAAAAAAGGCC |
| | | | | ACCTTTTATTAAGGTGGTCGGA |
| MG178 | 7246 | MG178- | nucleo- | AGTATCTATTTTGCCCTGATATCAGAAAAAAACGCT |
| recombinases | | 7207 AttR | tide | AAAAAAAGGATATAAAAAAAGAGATAACGCTCAGA |
| attachment | | | | CCGGCTATCGCGAACACCGCGGAAATACTGCCGTTC |
| sites | | | | AGAGGATACAAAAAAAGCCTCTTAATTTAAGAGGCT |
| | | | | TTTGATGATGGTGGGCCCTGCCTGACTCGAACAGGC |
| | | | | GACCAGACCGTTATGAGCGGCCTGCTCTAACCAACT |
| | | | | GAGCTAAGGGCCCGGCATCTGAAAACGGTTAAAAA |
| | | | | ATAGGTCAAATATTTTCCGAAGTCAAGCGCTTTTTGG |
| | | | | GCACCCCGCCGATCCGCGCCGC |
| MG178 | | MG178- | nucleo- | ATGGTGGGC |
| conserved | | 7207 Core | tide | |
| core | | | | |
| MG178 | 7248 | MG178- | nucleo- | GGGCTCCAACGGCCCATTTCCTTATCACGCCGATGTT |
| recombinases | | 7169 AttB | tide | CCTCCTCGTGCAGCTCTTTCCGGTGCGCTTGGCGGCA |
| attachment | | | | GAAGCCGCCAGGCACGACGCATCCGTGCGCCCAACA |
| sites | | | | AAAAAGCCTCACGAGGAGGCTTTCTTGCATCAAGAA |
| | | | | AGATCTGGTAGCGGGGGCAGGATTCGAACCTGCGAC |
| | | | | CTTCGGGTTATGAGCCCGACGAGCTGCCAGACTGCT |
| | | | | CCACCCCGCATCAGAGTCCGAAGAGTTTACCGGGGT |
| | | | | GGCCGGTTTCTTGCAAGCCTCAATCGTTCATCAATGG |
| | | | | CATCCATCGACCATTCAGAGACC |
| MG178 | 7249 | MG178- | nucleo- | GGGCTCCAACGGCCCATTTCCTTATCACGCCGATGTT |
| recombinases | | 7169 AttL | tide | CCTCCTCGTGCAGCTCTTTCCGGTGCGCTTGGCGGCA |
| attachment | | | | GAAGCCGCCAGGCACGACGCATCCGTGCGCCCAACA |
| sites | | | | AAAAAGCCTCACGAGGAGGCTTTCTTGCATCAAGAA |
| | | | | AGATCTGGTAGCGGGGGCGCGATGCGGTCTCTATCT |
| | | | | TACAGAGCCCTACCGCGTGCCTCTCTCCCGGCCAGA |
| | | | | GTCCGAGGATTGTACGGCAAAGCGCCGCCGCTGACA |
| | | | | ACACGTAGCGATATCCATTCTATGCATCGCACGGTG |
| | | | | AGACAGGCTGCCTGTACGCTTGAT |
| MG178 | 7250 | MG178- | nucleo- | TGGAACAACGTTGTCGCCCGCCTCACCGACACCCGC |
| recombinases | | 7169 AttP | tide | GACATCCCCGCCGCCCGCGACGCCCTGCGCGAACTC |
| attachment | | | | ATCGGCAACCGCGTAACCGTCAAAAACGAAAACGG |
| sites | | | | CGAACTCTTCGCAGAGATCGCCGCATCGGAATGTCA |
| | | | | GATAAAGCTGGTAGCGGGGGCGCGATGCGGTCTCTA |
| | | | | TCTTACAGAGCCCTACCGCGTGCCTCTCTCCCGGCCA |
| | | | | GAGTCCGAGGATTGTACGGCAAAGCGCCGCCGCTGA |
| | | | | CAACACGTAGCGATATCCATTCTATGCATCGCACGG |
| | | | | TGAGACAGGCTGCCTGTACGCTTGAT |
| MG178 | 7251 | MG178- | nucleo- | TGGAACAACGTTGTCGCCCGCCTCACCGACACCCGC |
| recombinases | | 7169 AttR | tide | GACATCCCCGCCGCCCGCGACGCCCTGCGCGAACTC |
| attachment | | | | ATCGGCAACCGCGTAACCGTCAAAAACGAAAACGG |
| sites | | | | CGAACTCTTCGCAGAGATCGCCGCATCGGAATGTCA |
| | | | | GATAAAGCTGGTAGCGGGGGCAGGATTCGAACCTGC |
| | | | | GACCTTCGGGTTATGAGCCCGACGAGCTGCCAGACT |
| | | | | GCTCCACCCCGCATCAGAGTCCGAAGAGTTTACCGG |
| | | | | GGTGGCCGGTTTCTTGCAAGCCTCAATCGTTCATCAA |
| | | | | TGGCATCCATCGACCATTCAGAGACC |
| MG178 | 7252 | MG178- | nucleo- | CTGGTAGCGGGGGC |
| conserved | | 7169 Core | tide | |
| core | | | | |
| MG178 | 7253 | MG178- | nucleo- | GTGGCAGTCCAAGGATTTCCTGTTGCATCCACATAG |
| recombinases | | 7170 AttB | tide | TAAAGGGCATTGTCATGGTCGGCATAATGCCCGCCC |
| attachment | | | | AGGCCTGCCCGCTCCATCTCTTCCGCAGAGGCCCCTT |
| sites | | | | TCGTCAGTTTATAGACCAAAGTGGCAATTATTTCTAA |
| | | | | ATGAGCCATTTCCTCAGTCCCTATATCCGTCAGTATG |
| | | | | GCCTTGGTTACATTGGTAGGCATACTGTACCGTTGAT |
| | | | | TTAAATAGCGTAATGCAGCTGACAGCTCCCCATCCG |
| | | | | GCCCGCCATATTGAGTGATCAAGTATTTGGCCATTCT |
| | | | | GAGATCGGGTTTGCT |
| MG178 | 7254 | MG178- | nucleo- | GTGGCAGTCCAAGGATTTCCTGTTGCATCCACATAG |
| recombinases | | 7170 AttL | tide | TAAAGGGCATTGTCATGGTCGGCATAATGCCCGCCC |
| attachment | | | | AGGCCTGCCCGCTCCATCTCTTCCGCAGAGGCCCCTT |
| sites | | | | TCGTCAGTTTATAGACCAAAGTGGCAATTATTTCTAA |
| | | | | ATGAGCCATTTCTTTTGCACCATAAGGTTATCTATAA |
| | | | | AAAACAACCCAGATTCAGGGGTTGTTTTTTTCGTATT |
| | | | | CATCCAGGTACTTTTCCACAATATACCTCAATTGCTC |
| | | | | CGAATTACTCCGGTGCTCCCTGGCTGCAACTTTAGTA |
| | | | | AACCTCTCATAAAG |
| MG178 | 7255 | MG178- | nucleo- | AGGAATCAGCTATTACACGAAATACTCGAAAAGGTG |
| recombinases | | 7170 AttP | tide | ATCTATACTAAAACCCAACGAGGTAATCGCGGAGGA |
| attachment | | | | CATGCAGATAATTTTAATCTAGTACTATATCCGAAG |
| sites | | | | CTAAACGTATCAAAGGATTTTAATTATTGATAACCTT |
| | | | | ATGGCGCCATTTCTTTTGCACCATAAGGTTATCTATA |
| | | | | AAAAACAACCCAGATTCAGGGGTTGTTTTTTTCGTAT |
| | | | | TCATCCAGGTACTTTTCCACAATATACCTCAATTGCT |
| | | | | CCGAATTACTCCGGTGCTCCCTGGCTGCAACTTTAGT |
| | | | | AAACCTCTCATAAAG |
| MG178 | 7256 | MG178- | nucleo- | AGGAATCAGCTATTACACGAAATACTCGAAAAGGTG |
| recombinases | | 7170 AttR | tide | ATCTATACTAAAACCCAACGAGGTAATCGCGGAGGA |
| attachment | | | | CATGCAGATAATTTTAATCTAGTACTATATCCGAAG |
| sites | | | | CTAAACGTATCAAAGGATTTTAATTATTGATAACCTT |
| | | | | ATGGCGCCATTTCCTCAGTCCCTATATCCGTCAGTAT |
| | | | | GGCCTTGGTTACATTGGTAGGCATACTGTACCGTTG |
| | | | | ATTTAAATAGCGTAATGCAGCTGACAGCTCCCCATC |
| | | | | CGGCCCGCCATATTGAGTGATCAAGTATTTGGCCAT |
| | | | | TCTGAGATCGGGTTTGCT |
| MG178 | | MG178- | nucleo- | GCCATTTC |
| conserved | | 7170 Core | tide | |
| core | | | | |
| MG178 | 7258 | MG178- | nucleo- | ACGTATGCAAAGCTTTGAGCATATGGGCGTATGATG |
| recombinases | | 7171 AttB | tide | AAGCTACTGAAACCATCCGGGTGTCCTTTCTCTGATG |
| attachment | | | | GCAGAGGGAATGTCAAATAAAGGACTATGATAGTA |
| sites | | | | GAAGAACAGGGAATTGATTTTCGGACAGGGGTTCGA |
| | | | | TTCCCCTCAGCTCCATGCTGAATGATGTCGGTTTTTC |
| | | | | CTTATTTTATAAGGAATTCCGGCATTTTCTTTTTAGA |
| | | | | AATGCTTGATAACCTTTTTAGGATACTGATTACCTTT |
| | | | | TTGATTACTTTTTAGGCTGTTTTCATTGGAATTGCAG |
| | | | | CCCGATTTATCTGCTGT |
| MG178 | 7259 | MG178- | nucleo- | ACGTATGCAAAGCTTTGAGCATATGGGCGTATGATG |
| recombinases | | 7171 AttL | tide | AAGCTACTGAAACCATCCGGGTGTCCTTTCTCTGATG |
| attachment | | | | GCAGAGGGAATGTCAAATAAAGGACTATGATAGTA |
| sites | | | | GAAGAACAGGGAATTGATTTTCGGACAGGGGTTCGA |
| | | | | TTCCCCTCAGCTCCACTTAAGGAGAATTATCCGAAC |
| | | | | ACTTTGTTTTATGCAAATGGAGTGTTTGGGATAATCG |
| | | | | TAAAGATTAACGGTAGGATTTGATTCCTACCGTTTTT |
| | | | | TTTTTGTGCGGGCCCGGTTCACCCGGTACCCGCCTTT |
| | | | | CACTTACTTCTCTTTCTC |
| MG178 | 7260 | MG178- | nucleo- | CAGACCCTGATTGACGTTTTTGTCAATGCAGTCTATG |
| recombinases | | 7171 AttP | tide | TCTATGATGACCGTATTGTAATCACGTACAATTATTC |
| attachment | | | | AGGGGCTCATAATTCGGTCACTTTGGAGCAGATTGA |
| sites | | | | ACAAGCCTTAGGGGAGTCTGAGGGTTCGGATACAGT |
| | | | | TTCGTCAGCTCCACTTAAGGAGAATTATCCGAACAC |
| | | | | TTTGTTTTATGCAAATGGAGTGTTTGGGATAATCGTA |
| | | | | AAGATTAACGGTAGGATTTGATTCCTACCGTTTTTTT |
| | | | | TTTGTGCGGGCCCGGTTCACCCGGTACCCGCCTTTCA |
| | | | | CTTACTTCTCTTTCTC |
| MG178 | 7261 | MG178- | nucleo- | CAGACCCTGATTGACGTTTTTGTCAATGCAGTCTATG |
| recombinases | | 7171 AttR | tide | TCTATGATGACCGTATTGTAATCACGTACAATTATTC |
| attachment | | | | AGGGGCTCATAATTCGGTCACTTTGGAGCAGATTGA |
| sites | | | | ACAAGCCTTAGGGGAGTCTGAGGGTTCGGATACAGT |
| | | | | TTCGTCAGCTCCATGCTGAATGATGTCGGTTTTTCCT |
| | | | | TATTTTATAAGGAATTCCGGCATTTTCTTTTTAGAAA |
| | | | | TGCTTGATAACCTTTTTAGGATACTGATTACCTTTTT |
| | | | | GATTACTTTTTAGGCTGTTTTCATTGGAATTGCAGCC |
| | | | | CGATTTATCTGCTGT |
| MG178 | | MG178- | nucleo- | TCAGCTCCA |
| conserved | | 7171 Core | tide | |
| core | | | | |
| MG178 | 7263 | MG178- | nucleo- | ATTGCGGTTTCAACTGAGAAGGACGAGCGCGGAATC |
| recombinases | | 7172 AttB 2 | tide | AACCTCGTTGCTCGCGGCTTTCGTTCGGCTGCTGTGT |
| attachment | | | | TTCGGTTGCGTCCTAGGCTGTGTTTTGCCTAGAACCG |
| sites | | | | CTACAGGCCACCGGCTGCGAAACCAACCGATGACCT |
| | | | | GCGATTCCCAATGGTGCCCCCGGCGCGATTCGAACG |
| | | | | CGCGGCACCCGCTTTAGGAGAGCGGTGCTCTGTCCC |
| | | | | CTGAGCTACGGAGGCGCGTTTGATATTGTAGCATTA |
| | | | | ATCGCCCTGCTGGCGCGGAACTTGCAAGACGCCTTG |
| | | | | CAGGACGCATAAAAAAGCGATGCGCC |
| MG178 | 7264 | MG178- | nucleo- | ATTGCGGTTTCAACTGAGAAGGACGAGCGCGGAATC |
| recombinases | | 7172 AttB 1 | tide | AACCTCGTTGCTCGCGGCTTTCGTTCGGCTGCTGTGT |
| attachment | | | | TTCGGTTGCGTCCTAGGCTGTGTTTTGCCTAGAACCG |
| sites | | | | CTACAGGCCACCGGCTGCGAAACCAACCGATGACCT |
| | | | | GCGATTCCAAATGGTGCCCCCGGCGCGATTCGAACG |
| | | | | CGCGGCACCCGCTTTAGGAGAGCGGTGCTCTGTCCC |
| | | | | CTGAGCTACGGAGGCGCGTTTGATATTGTAGCATTA |
| | | | | ATCGCCCTGCTGGCGCGGAACTTGCAAGACGCCTTG |
| | | | | CAGGACGCATAAAAAAGCGATGCGCC |
| MG178 | 7265 | MG178- | nucleo- | ATTGCGGTTTCAACTGAGAAGGACGAGCGCGGAATC |
| recombinases | | 7172 AttL | tide | AACCTCGTTGCTCGCGGCTTTCGTTCGGCTGCTGTGT |
| attachment | | | | TTCGGTTGCGTCCTAGGCTGTGTTTTGCCTAGAACCG |
| sites | | | | CTACAGGCCACCGGCTGCGAAACCAACCGATGACCT |
| | | | | GCGATTCCAAATGGTGCCCCAATCGCATGTCAAAAC |
| | | | | GAACGTCAAACGGGTATCCTTTTGAGACGTTGTACA |
| | | | | CCGAAAACGGAACGCCGATACTCATGCTTGAACATG |
| | | | | GTTTTGGGCTGGTCGTATCACTTAAAGCTTCTTGACG |
| | | | | AACTTGGACATGATGAAAAGGTCGT |
| MG178 | 7266 | MG178- | nucleo- | GATCACGCTTACTTTTGTATCTAAAGTTTGGCTGTAT |
| recombinases | | 7172 AttP 2 | tide | GAAAGCACCGCCGTGGCCGTGATGAACTTTGATTCG |
| attachment | | | | TGCGAAAGCACGCAATATGAGATAGAACTTGCTTTG |
| sites | | | | AAAAAACACGAACGGCCTGATCAACAGGCCGTTCGT |
| | | | | GAAACTTCCCAATGGTGCCCCAATCGCATGTCAAAA |
| | | | | CGAACGTCAAACGGGTATCCTTTTGAGACGTTGTAC |
| | | | | ACCGAAAACGGAACGCCGATACTCATGCTTGAACAT |
| | | | | GGTTTTGGGCTGGTCGTATCACTTAAAGCTTCTTGAC |
| | | | | GAACTTGGACATGATGAAAAGGTCGT |
| MG178 | 7267 | MG178- | nucleo- | GATCACGCTTACTTTTGTATCTAAAGTTTGGCTGTAT |
| recombinases | | 7172 AttP 1 | tide | GAAAGCACCGCCGTGGCCGTGATGAACTTTGATTCG |
| attachment | | | | TGCGAAAGCACGCAATATGAGATAGAACTTGCTTTG |
| sites | | | | AAAAAACACGAACGGCCTGATCAACAGGCCGTTCGT |
| | | | | GAAACTTCCAAATGGTGCCCCAATCGCATGTCAAAA |
| | | | | CGAACGTCAAACGGGTATCCTTTTGAGACGTTGTAC |
| | | | | ACCGAAAACGGAACGCCGATACTCATGCTTGAACAT |
| | | | | GGTTTTGGGCTGGTCGTATCACTTAAAGCTTCTTGAC |
| | | | | GAACTTGGACATGATGAAAAGGTCGT |
| MG178 | 7268 | MG178- | nucleo- | GATCACGCTTACTTTTGTATCTAAAGTTTGGCTGTAT |
| recombinases | | 7172 AttR | tide | GAAAGCACCGCCGTGGCCGTGATGAACTTTGATTCG |
| attachment | | | | TGCGAAAGCACGCAATATGAGATAGAACTTGCTTTG |
| sites | | | | AAAAAACACGAACGGCCTGATCAACAGGCCGTTCGT |
| | | | | GAAACTTCCAAATGGTGCCCCCGGCGCGATTCGAAC |
| | | | | GCGCGGCACCCGCTTTAGGAGAGCGGTGCTCTGTCC |
| | | | | CCTGAGCTACGGAGGCGCGTTTGATATTGTAGCATT |
| | | | | AATCGCCCTGCTGGCGCGGAACTTGCAAGACGCCTT |
| | | | | GCAGGACGCATAAAAAAGCGATGCGCC |
| MG178 | 7269 | MG178- | nucleo- | TTCCCAATGGTGCCCC |
| conserved | | 7172 Core 2 | tide | |
| core | | | | |
| MG178 | 7270 | MG178- | nucleo- | TTCCAAATGGTGCCCC |
| conserved | | 7172 Core 1 | tide | |
| core | | | | |
| MG178 | 7271 | MG178- | nucleo- | ATGGATCGCTACGCCTGGCTTGACAATCCCTGGCCC |
| recombinases | | 7174 AttB | tide | TGGGATTATCAGCCCCAGATGGAGGTATAAGAGATG |
| attachment | | | | TTTGTATATGAAAAGAAGCTCCAGTACCCTGTTAAG |
| sites | | | | ATAAAAAACACAAACCCTGCCCTTGCCAAATTCATT |
| | | | | ATTAGCCAATACGGCGGCCCTGACGGCGAGCTCGGC |
| | | | | GCTTCCCTTCGCTATCTAAGCCAGCGCTACTCAATGC |
| | | | | CATATCCAGAGCTGAAGGGTCTTCTGACGGATATCG |
| | | | | GCACGGAAGAGCTTGGGCATCTTGAGATGATAGGCG |
| | | | | CTATCGTTCATCAGCTGACAAG |
| MG178 | 7272 | MG178- | nucleo- | ATGGATCGCTACGCCTGGCTTGACAATCCCTGGCCC |
| recombinases | | 7174 AttL | tide | TGGGATTATCAGCCCCAGATGGAGGTATAAGAGATG |
| attachment | | | | TTTGTATATGAAAAGAAGCTCCAGTACCCTGTTAAG |
| sites | | | | ATAAAAAACACAAACCCTGCCCTTGCCAAATTCATT |
| | | | | ATTAGCCAATACGGCGGTGCTTATTGTAAAAACTTA |
| | | | | TTATATCTATTCTCCCAATTTTAAGCGCAGCAATCCT |
| | | | | TAATACGGTGCGGCTCGGCGCCGTATTAAGGATCGT |
| | | | | GCAATTACATATCAACGATTCGGCTCCGTGACAACT |
| | | | | GTCGCGATGGCGATAAGTATCC |
| MG178 | 7273 | MG178- | nucleo- | GTGCTTGATGTCCTTGATATAATCAAAAGCGACAAT |
| recombinases | | 7174 AttP | tide | GTATCTGAAGCTGACAAAAACACAGCTCTCAAAGCA |
| attachment | | | | ATTCTCAGTTATATTGTGTACGAAAAGGCTAATAAT |
| sites | | | | CGCCTCGCTTTGTATTTCTATTTTTGATATTATCGGTT |
| | | | | TTCACAATATGGCGGTGCTTATTGTAAAAACTTATTA |
| | | | | TATCTATTCTCCCAATTTTAAGCGCAGCAATCCTTAA |
| | | | | TACGGTGCGGCTCGGCGCCGTATTAAGGATCGTGCA |
| | | | | ATTACATATCAACGATTCGGCTCCGTGACAACTGTC |
| | | | | GCGATGGCGATAAGTATCC |
| MG178 | 7274 | MG178- | nucleo- | GTGCTTGATGTCCTTGATATAATCAAAAGCGACAAT |
| recombinases | | 7174 AttR | tide | GTATCTGAAGCTGACAAAAACACAGCTCTCAAAGCA |
| attachment | | | | ATTCTCAGTTATATTGTGTACGAAAAGGCTAATAAT |
| sites | | | | CGCCTCGCTTTGTATTTCTATTTTTGATATTATCGGTT |
| | | | | TTCACAATATGGCGGCCCTGACGGCGAGCTCGGCGC |
| | | | | TTCCCTTCGCTATCTAAGCCAGCGCTACTCAATGCCA |
| | | | | TATCCAGAGCTGAAGGGTCTTCTGACGGATATCGGC |
| | | | | ACGGAAGAGCTTGGGCATCTTGAGATGATAGGCGCT |
| | | | | ATCGTTCATCAGCTGACAAG |
| MG178 | 7275 | MG178- | nucleo- | CAATATGGCGG |
| conserved | | 7174 Core 1 | tide | |
| core | | | | |
| MG178 | 7276 | MG178- | nucleo- | CAATACGGCGG |
| conserved | | 7174 Core 2 | tide | |
| core | | | | |
| MG178 | 7277 | MG178- | nucleo- | TCCAGGCGGAGCGGGCAAACCCGGTTGGCAACTCGG |
| recombinases | | 7175 AttB | tide | GCGTTAATCAGACGTTCGGCAGGAGGAAAAGAGAC |
| attachment | | | | GGCTCGGTTTCTGCGTATCAACGGGATCGCCAGAAA |
| sites | | | | CGAAAAAAGCCTACAGATGTTGATCTGTAAGCCCTC |
| | | | | AAATTGTTGGTAGCGGGGGCAGGATTTGAACCTACG |
| | | | | ACCTTCGGGTTATGAGCCCGACGAGCTACCAGACTG |
| | | | | CTCCACCCCGCGTCAATTCAGAGTACTATACCGTAA |
| | | | | ATGCTTCCTCGATGCAATCCAACGTTTACTTTGGGTA |
| | | | | ACGAAACAGGCTCTTCGGATCTTCAGATTC |
| MG178 | 7278 | MG178- | nucleo- | TCCAGGCGGAGCGGGCAAACCCGGTTGGCAACTCGG |
| recombinases | | 7175 AttL | tide | GCGTTAATCAGACGTTCGGCAGGAGGAAAAGAGAC |
| attachment | | | | GGCTCGGTTTCTGCGTATCAACGGGATCGCCAGAAA |
| sites | | | | CGAAAAAAGCCTACAGATGTTGATCTGTAAGCCCTC |
| | | | | AAATTGTTGGTAGCGGGGGCAGGATACGTGCGTTAT |
| | | | | CTAACTCCGCTGCGCTGGCGGATATGGCCGCCGACC |
| | | | | TGATCAACCACGCCCGACACGAGCCCCGCCATTGGG |
| | | | | GCTTTTTTTATGCCTGAAAGAAACGGTTGAAGTCTCA |
| | | | | CGTATTAAAGGAATGACGGTCTTTTTGGCC |
| MG178 | 7279 | MG178- | nucleo- | TGGCCGAGCGACTGGAACGGATCGAAGACATCGCC |
| recombinases | | 7175 AttP | tide | GACGCGCGCGAAGCGCTCAGGAGCATTCTAGGGGA |
| attachment | | | | AGATATCAAGCTGGTGCCGGAGAACGGCGTATTGTG |
| sites | | | | GGCAGAACTCAAGGGCGGCTGCGCCGCTTTGAGTCA |
| | | | | GATAACGGTGGTAGCGGGGGCAGGATACGTGCGTTA |
| | | | | TCTAACTCCGCTGCGCTGGCGGATATGGCCGCCGAC |
| | | | | CTGATCAACCACGCCCGACACGAGCCCCGCCATTGG |
| | | | | GGCTTTTTTTATGCCTGAAAGAAACGGTTGAAGTCTC |
| | | | | ACGTATTAAAGGAATGACGGTCTTTTTGGCC |
| MG178 | 7280 | MG178- | nucleo- | TGGCCGAGCGACTGGAACGGATCGAAGACATCGCC |
| recombinases | | 7175 AttR | tide | GACGCGCGCGAAGCGCTCAGGAGCATTCTAGGGGA |
| attachment | | | | AGATATCAAGCTGGTGCCGGAGAACGGCGTATTGTG |
| sites | | | | GGCAGAACTCAAGGGCGGCTGCGCCGCTTTGAGTCA |
| | | | | GATAACGGTGGTAGCGGGGGCAGGATTTGAACCTAC |
| | | | | GACCTTCGGGTTATGAGCCCGACGAGCTACCAGACT |
| | | | | GCTCCACCCCGCGTCAATTCAGAGTACTATACCGTA |
| | | | | AATGCTTCCTCGATGCAATCCAACGTTTACTTTGGGT |
| | | | | AACGAAACAGGCTCTTCGGATCTTCAGATTC |
| MG178 | 7281 | MG178- | nucleo- | TGGTAGCGGGGGCAGGAT |
| conserved | | 7175 Core | tide | |
| core | | | | |
| MG178 | 7282 | MG178- | nucleo- | CGAGTCTCGCGTCGGGAGCCCCATCTGCGGCCGGAA |
| recombinases | | 7176 AttB | tide | TCGCCGCCGCGGCATCCAGAGGCAGCGGATCCCAGT |
| attachment | | | | GCCTCTGTCGGAGGAGCCGATGGGCCTGGTGGCGGA |
| sites | | | | CGATGCTGCGGAACCAGCCGGGGAACGCGGCCGGA |
| | | | | TCGGCCAGGGTCCGCAGGCCGAACCACGCGGCGAC |
| | | | | GAAAGACTCCTGGACGACGTCCTCCGCCTGTTGTAG |
| | | | | GTCACGCAGGAGAGCCAGGGCGTAGCCGAAGGCCA |
| | | | | TCTGCTGAAAGCGCCGCGTCACCTCCGCGAACGCGT |
| | | | | CCAGGTCGCCCTGCCTCGCC |
| MG178 | 7283 | MG178- | nucleo- | CGAGTCTCGCGTCGGGAGCCCCATCTGCGGCCGGAA |
| recombinases | | 7176 AttL | tide | TCGCCGCCGCGGCATCCAGAGGCAGCGGATCCCAGT |
| attachment | | | | GCCTCTGTCGGAGGAGCCGATGGGCCTGGTGGCGGA |
| sites | | | | CGATGCTGCGGAACCAGCCGGGGAACGCGGCCGGA |
| | | | | TCGGCCAGGGTCAGGAGACTCACCCGCGTGAACCCG |
| | | | | TCCAAGGTGGCCAAGCGCTCGATCGGGAAGGATCCC |
| | | | | ACAAGTTCCCTAGCGTGGTTCGGGGCCAGCGCGCTC |
| | | | | GACCTCGTTGTGTCGATCAGTGGTCAGGTACACAGA |
| | | | | CGTGCCGCATAGTGTTGC |
| MG178 | 7284 | MG178- | nucleo- | TGAAGAAGGTCTTGCCGAGCAAGTTGATTCTGACTC |
| recombinases | | 7176 AttP | tide | CCCATCCCGAGAGTCGGTCCTACTCGTTCGCTGGCG |
| attachment | | | | ACGGAGCCATCGGCCCTCTGCTGGGACAGGTGGTCT |
| sites | | | | CGTCTCAGATGGGGAAGGGCCAGGGTTCAAGTGGG |
| | | | | GAAGGTCGGGTCAGGAGACTCACCCGCGTGAACCCG |
| | | | | TCCAAGGTGGCCAAGCGCTCGATCGGGAAGGATCCC |
| | | | | ACAAGTTCCCTAGCGTGGTTCGGGGCCAGCGCGCTC |
| | | | | GACCTCGTTGTGTCGATCAGTGGTCAGGTACACAGA |
| | | | | CGTGCCGCATAGTGTTGC |
| MG178 | 7285 | MG178- | nucleo- | TGAAGAAGGTCTTGCCGAGCAAGTTGATTCTGACTC |
| recombinases | | 7176 AttR | tide | CCCATCCCGAGAGTCGGTCCTACTCGTTCGCTGGCG |
| attachment | | | | ACGGAGCCATCGGCCCTCTGCTGGGACAGGTGGTCT |
| sites | | | | CGTCTCAGATGGGGAAGGGCCAGGGTTCAAGTGGG |
| | | | | GAAGGTCGGGTCCGCAGGCCGAACCACGCGGCGAC |
| | | | | GAAAGACTCCTGGACGACGTCCTCCGCCTGTTGTAG |
| | | | | GTCACGCAGGAGAGCCAGGGCGTAGCCGAAGGCCA |
| | | | | TCTGCTGAAAGCGCCGCGTCACCTCCGCGAACGCGT |
| | | | | CCAGGTCGCCCTGCCTCGCC |
| MG178 | | MG178- | nucleo- | GGGTC |
| conserved | | 7176 Core | tide | |
| core | | | | |
| MG178 | 7287 | MG178- | nucleo- | TTTAACTTCTTTATCCTCTGTTTCATCAATCACTGCAT |
| recombinases | | 7178 AttB | tide | TCATAATGATATTTTCATTGAATGTACTTGCCGTTTC |
| attachment | | | | TGCAACTGGCATAGAATACTCCCAATTTAATGGTCT |
| sites | | | | ATGACTTTCAATGTTTAATCCATGATATGCATGTCCA |
| | | | | AGTTCATGAGCCAAAGTAACTACGTCTCCAAGTCCT |
| | | | | CCATCAAAATTAGTTAATACGCGTGATTGTTTCGCA |
| | | | | AACGGCATATTGCAGCAGAAAGCTCCACCAACTTTT |
| | | | | CCTTTATGTGGATAAAAATCAATCCAGTTATTGTCAA |
| | | | | ATGCTTCTTCCATCA |
| MG178 | 7288 | MG178- | nucleo- | TTTAACTTCTTTATCCTCTGTTTCATCAATCACTGCAT |
| recombinases | | 7178 AttL | tide | TCATAATGATATTTTCATTGAATGTACTTGCCGTTTC |
| attachment | | | | TGCAACTGGCATAGAATACTCCCAATTTAATGGTCT |
| sites | | | | ATGACTTTCAATGTTTAATCCATGATATGCATGTCCA |
| | | | | AGTTCATGAGAGCGTGGATGTATACGCAAAATAAAT |
| | | | | AAGCGTACTGTTTAGGAAAATAAAAAAATAGCCCTG |
| | | | | TTTCAACAGAGCTACCATAGATTAATGTCAAAATGT |
| | | | | ATATTAACCTCAAAGCTTTTCCATTATAGCATAAAA |
| | | | | AAAAGAACCTATGGAA |
| MG178 | 7289 | MG178- | nucleo- | ATTGAAATGCTAAAGGATGATTCTATATCCGCAAAA |
| recombinases | | 7178 AttP | tide | ATAAAAAATAATTTCCTAAAGGAAATCATACAAGTT |
| attachment | | | | ATTTATTATAAAAAAGATAAATTAGGAAATATTACA |
| sites | | | | TTAGATATTTATTTACGCTAGTAGCGAGCATTCACGG |
| | | | | ACTGATTCATGAGAGCGTGGATGTATACGCAAAATA |
| | | | | AATAAGCGTACTGTTTAGGAAAATAAAAAAATAGCC |
| | | | | CTGTTTCAACAGAGCTACCATAGATTAATGTCAAAA |
| | | | | TGTATATTAACCTCAAAGCTTTTCCATTATAGCATAA |
| | | | | AAAAAAGAACCTATGGAA |
| MG178 | 7290 | MG178- | nucleo- | ATTGAAATGCTAAAGGATGATTCTATATCCGCAAAA |
| recombinases | | 7178 AttR | tide | ATAAAAAATAATTTCCTAAAGGAAATCATACAAGTT |
| attachment | | | | ATTTATTATAAAAAAGATAAATTAGGAAATATTACA |
| sites | | | | TTAGATATTTATTTACGCTAGTAGCGAGCATTCACGG |
| | | | | ACTGATTCATGAGCCAAAGTAACTACGTCTCCAAGT |
| | | | | CCTCCATCAAAATTAGTTAATACGCGTGATTGTTTCG |
| | | | | CAAACGGCATATTGCAGCAGAAAGCTCCACCAACTT |
| | | | | TTCCTTTATGTGGATAAAAATCAATCCAGTTATTGTC |
| | | | | AAATGCTTCTTCCATCA |
| MG178 | | MG178- | nucleo- | TTCATGAG |
| conserved | | 7178 Core | tide | |
| core | | | | |
| MG178 | 7292 | MG178- | nucleo- | AAGGTAAGCTCGCCAGTCCTCCGACGCGCTGCGCGC |
| recombinases | | 7179 AttB | tide | TATGGAGGACATCCTTCCCCTGCGGGTACTCCTGTCT |
| attachment | | | | GCGCTCATCCTTTTCACTCGCTTTGCTCGCGGAAAAG |
| sites | | | | AATGGCTTGCCATCCGTAGCTCGACTTCGTCGAGCG |
| | | | | AAGGATGGTGGGCGTAACAGAACTCGAATCTGTGAC |
| | | | | CTTCACGATGTCAACGTGACGCTCTAACCAACTGAG |
| | | | | CTATACGCCCTTGCGGACTTGAAACACAATCCGGAC |
| | | | | CGGCGGGCGGCCGGATTCTTTTGCGGTTGCGGCGTT |
| | | | | TTACATAACCACCGACAAC |
| MG178 | 7293 | MG178- | nucleo- | AAGGTAAGCTCGCCAGTCCTCCGACGCGCTGCGCGC |
| recombinases | | 7179 AttL | tide | TATGGAGGACATCCTTCCCCTGCGGGTACTCCTGTCT |
| attachment | | | | GCGCTCATCCTTTTCACTCGCTTTGCTCGCGGAAAAG |
| sites | | | | AATGGCTTGCCATCCGTAGCTCGACTTCGTCGAGCG |
| | | | | AAGGATGGTGGGCCAAATCAAACTTACTCATAACAA |
| | | | | GATTTTTGGAATTTAAATGCGGGAGGCGGGGCCAGC |
| | | | | TTCGCGCATCGCAAGTATGACATTTCAAAAAAAAGG |
| | | | | CAAAAAATTCCCGGCCGAATTTTGGCAGCGGGGAGA |
| | | | | GCTGCGCCGCCTCTGATCC |
| MG178 | 7294 | MG178- | nucleo- | AGAATGGACAAAAAAAATTGCATCTGGTGAAGCTG |
| recombinases | | 7179 AttP | tide | ATTTTGCTACCAAACGCAATTTAATCATGTCGGTAAT |
| attachment | | | | CGAATCTCTTGAATGCGTCAAACGATCAGATACTCA |
| sites | | | | AATAGGCTTCAAGATGAAACTTGTTATGAGTAGTAG |
| | | | | TTGTAAATGGTGGGCCAAATCAAACTTACTCATAAC |
| | | | | AAGATTTTTGGAATTTAAATGCGGGAGGCGGGGCCA |
| | | | | GCTTCGCGCATCGCAAGTATGACATTTCAAAAAAAA |
| | | | | GGCAAAAAATTCCCGGCCGAATTTTGGCAGCGGGGA |
| | | | | GAGCTGCGCCGCCTCTGATCC |
| MG178 | 7295 | MG178- | nucleo- | AGAATGGACAAAAAAAATTGCATCTGGTGAAGCTG |
| recombinases | | 7179 AttR | tide | ATTTTGCTACCAAACGCAATTTAATCATGTCGGTAAT |
| attachment | | | | CGAATCTCTTGAATGCGTCAAACGATCAGATACTCA |
| sites | | | | AATAGGCTTCAAGATGAAACTTGTTATGAGTAGTAG |
| | | | | TTGTAAATGGTGGGCGTAACAGAACTCGAATCTGTG |
| | | | | ACCTTCACGATGTCAACGTGACGCTCTAACCAACTG |
| | | | | AGCTATACGCCCTTGCGGACTTGAAACACAATCCGG |
| | | | | ACCGGCGGGCGGCCGGATTCTTTTGCGGTTGCGGCG |
| | | | | TTTTACATAACCACCGACAAC |
| MG178 | | MG178- | nucleo- | ATGGTGGGC |
| conserved | | 7179 Core | tide | |
| core | | | | |
| MG178 | 7297 | MG178- | nucleo- | TAAAAATATGTCTTGACCTGAACGATATTTGCATAT |
| recombinases | | 7180 AttB | tide | ATACATTCGTTAGTTTTTGGCAGATTGCTGCTGAAAA |
| attachment | | | | GTCCTAACGGTCCCATCGTCTAATGGTTAGGACACC |
| sites | | | | ACCCTTTCACGGTGGCGATACGAGTTCGAATCTCGTT |
| | | | | GGGATCACCAATGCAAAGAAACCTCCCTTGCGGAGG |
| | | | | TTTCTTTGTATTGATAAAGAGCCCAGGAGAGATTTG |
| | | | | AACGAGTAGAGTTCGGAGCGGGTAGTTGGGGAGCG |
| | | | | CGATTAAAAGAAGCGCGGGCCGTACGTCGCGAAAG |
| | | | | GGGCCCGCGGTGGGAGCCGTGGGA |
| MG178 | 7298 | MG178- | nucleo- | TAAAAATATGTCTTGACCTGAACGATATTTGCATAT |
| recombinases | | 7180 AttL | tide | ATACATTCGTTAGTTTTTGGCAGATTGCTGCTGAAAA |
| attachment | | | | GTCCTAACGGTCCCATCGTCTAATGGTTAGGACACC |
| sites | | | | ACCCTTTCACGGTGGCGATACGAGTTCGAATCTCGTT |
| | | | | GGGATCACCAATGCAAGCGGCAGGTATAAAAACTG |
| | | | | CCGTTTTTCTTTTATAAATCAAGCACTTAGCCGAGTT |
| | | | | CGAATGTTGTAAATTTGAAAATGTCATTTTGGGAGA |
| | | | | ATTTTCCGAACTCGTTTGACGATAATCCTTTATTTTT |
| | | | | CAATGGTTTGTCTGAGTTAGA |
| MG178 | 7299 | MG178- | nucleo- | GAAAAGTTGAAGGAACAGATATTCCCGAAACCCTTA |
| recombinases | | 7180 AttP | tide | CGCTTAATAGTGATAATGAAATTATTAAAATTGAAG |
| attachment | | | | ATATGTTGAAAGAAATTGAAAAGTTTTGGGATAATA |
| sites | | | | AAATTTTATCATCTAAATAGCTCGTGGATTGTGAGTT |
| | | | | AAGCTTCACCAATACAAGCGGCAGGTATAAAAACTG |
| | | | | CCGTTTTTCTTTTATAAATCAAGCACTTAGCCGAGTT |
| | | | | CGAATGTTGTAAATTTGAAAATGTCATTTTGGGAGA |
| | | | | ATTTTCCGAACTCGTTTGACGATAATCCTTTATTTTT |
| | | | | CAATGGTTTGTCTGAGTTAGA |
| MG178 | 7300 | MG178- | nucleo- | GAAAAGTTGAAGGAACAGATATTCCCGAAACCCTTA |
| recombinases | | 7180 AttR | tide | CGCTTAATAGTGATAATGAAATTATTAAAATTGAAG |
| attachment | | | | ATATGTTGAAAGAAATTGAAAAGTTTTGGGATAATA |
| sites | | | | AAATTTTATCATCTAAATAGCTCGTGGATTGTGAGTT |
| | | | | AAGCTTCACCAATACAAAGAAACCTCCCTTGCGGAG |
| | | | | GTTTCTTTGTATTGATAAAGAGCCCAGGAGAGATTT |
| | | | | GAACGAGTAGAGTTCGGAGCGGGTAGTTGGGGAGC |
| | | | | GCGATTAAAAGAAGCGCGGGCCGTACGTCGCGAAA |
| | | | | GGGGCCCGCGGTGGGAGCCGTGGGA |
| MG178 | 7301 | MG178- | nucleo- | TCACCAATGCAA |
| conserved | | 7180 Core | tide | |
| core | | | | |
| MG178 | 7302 | MG178- | nucleo- | TTAAGAAAGCTTAAGAGGCCAGAAGCTCCTTACAGT |
| recombinases | | 7181 AttB | tide | TGGACCCATCTGAACCGAATTTTGCTAAAAGCAGAC |
| attachment | | | | GAAAAAACGGGAACGCCGTTCTTTTGCTCCCTAAAA |
| sites | | | | GATCAAGCCTCCTGTCCATGGACAGGAGGCTTGCAA |
| | | | | GTTATACAGTTGGTGGGCCCACTAGGATTCGAACCT |
| | | | | AGGACCAACCGGTTATGAGCCGGGGGCTCTACCGCT |
| | | | | GAGCTATAGGCCCAATGTAGGAATTATACCCCAGAA |
| | | | | AAAGATGCGCTGTCAACGTTTCTTACTCGAGGAATT |
| | | | | CCTTGAGGGGTTTGCTCCGTTTCG |
| MG178 | 7303 | MG178- | nucleo- | TTAAGAAAGCTTAAGAGGCCAGAAGCTCCTTACAGT |
| recombinases | | 7181 AttL | tide | TGGACCCATCTGAACCGAATTTTGCTAAAAGCAGAC |
| attachment | | | | GAAAAAACGGGAACGCCGTTCTTTTGCTCCCTAAAA |
| sites | | | | GATCAAGCCTCCTGTCCATGGACAGGAGGCTTGCAA |
| | | | | GTTATACAGTTGGTGGGCTATCTAACCCTGAGTGCG |
| | | | | AACCTAAAAGTAAAGATAGGCCGCGGCTGGTTTTCC |
| | | | | GTATCCTACCCCATCAGTACACAGGGGATTTGATAT |
| | | | | TTTATTCTAGGAGAAGGCAATTACTTTTGACCTGACT |
| | | | | TTTGAAGAGCTAGCCGAATGCCG |
| MG178 | 7304 | MG178- | nucleo- | CAGATGTACCGGAACAATTTACTTGATGTCCTGGTT |
| recombinases | | 7181 AttP | tide | AGTCGTGTTGTCCTTTACCCCAACAGGGCAGAAGTA |
| attachment | | | | TTTTATCGCTACAAAAAAGAACTCCCTTCCCTCCCTA |
| sites | | | | ATCCAGTTATCATCAGGGAAGAAAGGGGTTCGAATG |
| | | | | GCACGCAGTTGGTGGGCTATCTAACCCTGAGTGCGA |
| | | | | ACCTAAAAGTAAAGATAGGCCGCGGCTGGTTTTCCG |
| | | | | TATCCTACCCCATCAGTACACAGGGGATTTGATATTT |
| | | | | TATTCTAGGAGAAGGCAATTACTTTTGACCTGACTTT |
| | | | | TGAAGAGCTAGCCGAATGCCG |
| MG178 | 7305 | MG178- | nucleo- | CAGATGTACCGGAACAATTTACTTGATGTCCTGGTT |
| recombinases | | 7181 AttR | tide | AGTCGTGTTGTCCTTTACCCCAACAGGGCAGAAGTA |
| attachment | | | | TTTTATCGCTACAAAAAAGAACTCCCTTCCCTCCCTA |
| sites | | | | ATCCAGTTATCATCAGGGAAGAAAGGGGTTCGAATG |
| | | | | GCACGCAGTTGGTGGGCCCACTAGGATTCGAACCTA |
| | | | | GGACCAACCGGTTATGAGCCGGGGGCTCTACCGCTG |
| | | | | AGCTATAGGCCCAATGTAGGAATTATACCCCAGAAA |
| | | | | AAGATGCGCTGTCAACGTTTCTTACTCGAGGAATTC |
| | | | | CTTGAGGGGTTTGCTCCGTTTCG |
| MG178 | 7306 | MG178- | nucleo- | CAGTTGGTGGGC |
| conserved | | 7181 Core | tide | |
| core | | | | |
| MG178 | 7307 | MG178- | nucleo- | ATTTGTATTGAAATAAAGTACAGCAAAGAAAAGAA |
| recombinases | | 7182 AttB | tide | GATTTTTTGATATAAATACTGATATCGAATAAAAAA |
| attachment | | | | GAGAGTACAAGGTTATAGTCCTTGTACTCTCTTTTTT |
| sites | | | | ATTCGATAACAAATACGAACTTATTCTTCGTCTCCGT |
| | | | | TTTCGTCTTCCTTCATATTGTGGAACACATTCTGAAC |
| | | | | GTCTTCGTCCTCTTCCAACTTTTCAATCAATTTTTCAA |
| | | | | TCGATTCACGCTGCTCGGGAGTCACTTCTTTCACATC |
| | | | | GTTCGGAATACGGACAAATTCGCTACTGGTGATTTC |
| | | | | ATATCCATTCTCTTCCAG |
| MG178 | 7308 | MG178- | nucleo- | ATTTGTATTGAAATAAAGTACAGCAAAGAAAAGAA |
| recombinases | | 7182 AttL | tide | GATTTTTTGATATAAATACTGATATCGAATAAAAAA |
| attachment | | | | GAGAGTACAAGGTTATAGTCCTTGTACTCTCTTTTTT |
| sites | | | | ATTCGATAACAAATACGAACTTATTCTTCGTCTCCGT |
| | | | | TTTCGTCTTCCTTCATTACAATGGAAAAGTTTGCAAT |
| | | | | CTTAATACCTTCATTGTATATTTGCATACAAATAAAT |
| | | | | GATATTCAAGATGAAAAAAGCAGCTTTATTATTAAG |
| | | | | ATGTAGTACTGATTCACAGGATTATGATAGGCAGCA |
| | | | | AAGAGATTTACTACCTACGG |
| MG178 | 7309 | MG178- | nucleo- | TGGTAAGTTCCTAAAGTCCAAAACTCCATTACTACA |
| recombinases | | 7182 AttP | tide | AAAAACAAAAGGAAGCACAAATCAGCCTCCTTTTGT |
| attachment | | | | TAAATTATACTAAAATATAATATACTAATATTTATAA |
| sites | | | | TATATTGATATTCAAATTATTCTTCATTGTCATTAAT |
| | | | | TCCATCTTCCTTCATTACAATGGAAAAGTTTGCAATC |
| | | | | TTAATACCTTCATTGTATATTTGCATACAAATAAATG |
| | | | | ATATTCAAGATGAAAAAAGCAGCTTTATTATTAAGA |
| | | | | TGTAGTACTGATTCACAGGATTATGATAGGCAGCAA |
| | | | | AGAGATTTACTACCTACGG |
| MG178 | 7310 | MG178- | nucleo- | TGGTAAGTTCCTAAAGTCCAAAACTCCATTACTACA |
| recombinases | | 7182 AttR | tide | AAAAACAAAAGGAAGCACAAATCAGCCTCCTTTTGT |
| attachment | | | | TAAATTATACTAAAATATAATATACTAATATTTATAA |
| sites | | | | TATATTGATATTCAAATTATTCTTCATTGTCATTAAT |
| | | | | TCCATCTTCCTTCATATTGTGGAACACATTCTGAACG |
| | | | | TCTTCGTCCTCTTCCAACTTTTCAATCAATTTTTCAAT |
| | | | | CGATTCACGCTGCTCGGGAGTCACTTCTTTCACATCG |
| | | | | TTCGGAATACGGACAAATTCGCTACTGGTGATTTCA |
| | | | | TATCCATTCTCTTCCAG |
| MG178 | 7311 | MG178- | nucleo- | TCTTCCTTCAT |
| conserved | | 7182 Core | tide | |
| core | | | | |
| MG178 | 7312 | MG178- | nucleo- | GGCTAAGAAGGATGGTGAGATAAAAGATGCCCTCTC |
| recombinases | | 7183 AttB | tide | CTATATCAGGGAAACCGGCGCAGAGTTTGACCTGGA |
| attachment | | | | CCTCCCAGCCAAGGGTGTTCCGACCAAGGGCCTCAG |
| sites | | | | ATCAGACCGGTTAGGGGTTAAGAAGTCAGAGGAGG |
| | | | | CGGGCCGATGATGTTCAGATTCGCCTACCCGGTCGT |
| | | | | GCTCCTGCTTCTCCTTGTTGTGGCGGGATGGCTTTTC |
| | | | | TTTGCCCTATGGAGGAAGCCTTCCGGCATCACCTATT |
| | | | | CCATGACCTCAAAGATGGCTGGCCTTGCCGGGGGTG |
| | | | | TGAACCAGGTCCTGGCGAGGC |
| MG178 | 7313 | MG178- | nucleo- | GGCTAAGAAGGATGGTGAGATAAAAGATGCCCTCTC |
| recombinases | | 7183 AttL | tide | CTATATCAGGGAAACCGGCGCAGAGTTTGACCTGGA |
| attachment | | | | CCTCCCAGCCAAGGGTGTTCCGACCAAGGGCCTCAG |
| sites | | | | ATCAGACCGGTTAGGGGTTAAGAAGTCAGAGGAGG |
| | | | | CGGGCCGATGATGTTCATGTTGTAGACACTTTACAA |
| | | | | CTATCAACACATAATCAAAAATAATCATTGACAAGG |
| | | | | CATTTTCAAAGATGGTATAAGGTGAAAATGAAAGCA |
| | | | | AATCGAAGCTGTTAGTAGTAATTGGCTGATTATCGA |
| | | | | AGGAAATGAGGACATTTACTCTG |
| MG178 | 7314 | MG178- | nucleo- | AGGAGATCCTAATTATGTGATCTCATTCAAGAGCGG |
| recombinases | | 7183 AttP | tide | TGTCATTAGATCTCTATTCCCTTACAAAGATAAGAA |
| attachment | | | | GTTATCATGGCAATTAGACAAGACAAAGAAACAACT |
| sites | | | | GGACATCTATTAATGATAACAGCAATGGGTTAAATA |
| | | | | GACTATATGATGTTCATGTTGTAGACACTTTACAACT |
| | | | | ATCAACACATAATCAAAAATAATCATTGACAAGGCA |
| | | | | TTTTCAAAGATGGTATAAGGTGAAAATGAAAGCAAA |
| | | | | TCGAAGCTGTTAGTAGTAATTGGCTGATTATCGAAG |
| | | | | GAAATGAGGACATTTACTCTG |
| MG178 | 7315 | MG178- | nucleo- | AGGAGATCCTAATTATGTGATCTCATTCAAGAGCGG |
| recombinases | | 7183 AttR | tide | TGTCATTAGATCTCTATTCCCTTACAAAGATAAGAA |
| attachment | | | | GTTATCATGGCAATTAGACAAGACAAAGAAACAACT |
| sites | | | | GGACATCTATTAATGATAACAGCAATGGGTTAAATA |
| | | | | GACTATATGATGTTCAGATTCGCCTACCCGGTCGTGC |
| | | | | TCCTGCTTCTCCTTGTTGTGGCGGGATGGCTTTTCTTT |
| | | | | GCCCTATGGAGGAAGCCTTCCGGCATCACCTATTCC |
| | | | | ATGACCTCAAAGATGGCTGGCCTTGCCGGGGGTGTG |
| | | | | AACCAGGTCCTGGCGAGGC |
| MG178 | 7316 | MG178- | nucleo- | ATGATGTTCA |
| conserved | | 7183 Core | tide | |
| core | | | | |
| MG178 | 7317 | MG178- | nucleo- | TGGAACGGCACGAAGCGCCTTGGGAGGATTCAAGGT |
| recombinases | | 7184 AttB | tide | CATGGGGGGCCTGGTCTCGGCCAATATGACCTATAA |
| attachment | | | | TGCCAAAGAAAACAAATCCGTCATCGACAGCTCCAA |
| sites | | | | TAAACCCCGCGGCGGGTTCGGCGGCGGCCTGGGATA |
| | | | | TGAAAGGGGATCCCGACTCGGGTTTGAAATCGATTT |
| | | | | GCTCTATCTCCCGAAAGGGGCGCTTTTCAAGGGGGA |
| | | | | TTTATCCGGGATCACCTTTGATTTGAAGTTCAGCATC |
| | | | | GACGAAGTGAGCGTCCCCATCCTTCTCAAGCTGAAT |
| | | | | GTCCTCAGGAACAAAGTGC |
| MG178 | 7318 | MG178- | nucleo- | TGGAACGGCACGAAGCGCCTTGGGAGGATTCAAGGT |
| recombinases | | 7184 AttL | tide | CATGGGGGGCCTGGTCTCGGCCAATATGACCTATAA |
| attachment | | | | TGCCAAAGAAAACAAATCCGTCATCGACAGCTCCAA |
| sites | | | | TAAACCCCGCGGCGGGTTCGGCGGCGGCCTGGGATA |
| | | | | TGAAAGGGGATCCCAAAATATATGAACTTATAAATG |
| | | | | GGTTTGTAACCTTCCTCAAGCATTTCAGCGTATCCCC |
| | | | | TCAACTCCAAAACCTTTGTCAGATCAGAGGGTAAAA |
| | | | | AATGCCTACATCCCTGAGAAAGAGAATTGACCGGGA |
| | | | | CATGGAGCAGAAGGAAGAA |
| MG178 | 7319 | MG178- | nucleo- | AAATCCTTGCTCTCGTTCTTGATAAGGTTTTTCTAAG |
| recombinases | | 7184 AttP | tide | AGACGGCAAAGCACAATTCCACTGGAAACCTCCGTT |
| attachment | | | | CGACTTTCTATTTTCGATAAACAAAATCTTAGAAGA |
| sites | | | | AGAAGAACCCCAGGCAGGTTCTTTTATAAGGTCGAG |
| | | | | TGAAAGGGATCCCAAAATATATGAACTTATAAATGG |
| | | | | GTTTGTAACCTTCCTCAAGCATTTCAGCGTATCCCCT |
| | | | | CAACTCCAAAACCTTTGTCAGATCAGAGGGTAAAAA |
| | | | | ATGCCTACATCCCTGAGAAAGAGAATTGACCGGGAC |
| | | | | ATGGAGCAGAAGGAAGAA |
| MG178 | 7320 | MG178- | nucleo- | AAATCCTTGCTCTCGTTCTTGATAAGGTTTTTCTAAG |
| recombinases | | 7184 AttR | tide | AGACGGCAAAGCACAATTCCACTGGAAACCTCCGTT |
| attachment | | | | CGACTTTCTATTTTCGATAAACAAAATCTTAGAAGA |
| sites | | | | AGAAGAACCCCAGGCAGGTTCTTTTATAAGGTCGAG |
| | | | | TGAAAGGGATCCCGACTCGGGTTTGAAATCGATTTG |
| | | | | CTCTATCTCCCGAAAGGGGCGCTTTTCAAGGGGGAT |
| | | | | TTATCCGGGATCACCTTTGATTTGAAGTTCAGCATCG |
| | | | | ACGAAGTGAGCGTCCCCATCCTTCTCAAGCTGAATG |
| | | | | TCCTCAGGAACAAAGTGC |
| MG178 | | MG178- | nucleo- | GGGATCCC |
| conserved | | 7184 Core | tide | |
| core | | | | |
| MG178 | 7322 | MG178- | nucleo- | GGAAAATCTAATGCCTTCAGAGAACTCTGCAAAATT |
| recombinases | | 7185 AttB | tide | TCGGCCTGAAAAGTTCTCTATGGAGCTCGTAGAAAT |
| attachment | | | | GGTCCAGGTGCAAAAACCTCGCGATTTGGCGGGCTT |
| sites | | | | TCCGGCCAAACTTATGGGCCGCACGTTTCGCTGAGG |
| | | | | ACTGTTTGGTAGCGGGGGAGGGACTCGAACCCCCGA |
| | | | | CACGCGGATTATGATTCCGCTGCTCTAACCAGCTGA |
| | | | | GCTACCCCGCCGCCACCAGCCCGGCGAAACCGGGCA |
| | | | | TTTCAACCAACCATATAAGGGGTTGGGCGCCGGTTC |
| | | | | TAAAGGCGTGCGAGCCGGTGTCAA |
| MG178 | 7323 | MG178- | nucleo- | GGAAAATCTAATGCCTTCAGAGAACTCTGCAAAATT |
| recombinases | | 7185 AttL | tide | TCGGCCTGAAAAGTTCTCTATGGAGCTCGTAGAAAT |
| attachment | | | | GGTCCAGGTGCAAAAACCTCGCGATTTGGCGGGCTT |
| sites | | | | TCCGGCCAAACTTATGGGCCGCACGTTTCGCTGAGG |
| | | | | ACTGTTTGGTAGCGGGGGCTCGTCTCATTCAATCCCA |
| | | | | CCGACTGGCCAAGTGATACCCTTTAGGCTGACTTGC |
| | | | | CGGGGTCCGCTGGCGGCATGACCTACTGCCACTTCT |
| | | | | CCATGCGAGTAACTGCCGCCGCGGGCACCACGATGG |
| | | | | TCTTGCGGTCAAAGTAGAATACT |
| MG178 | 7324 | MG178- | nucleo- | GGCCATCCGCGAGCTAGTGGAGACGGTTACCGTCAG |
| recombinases | | 7185 AttP | tide | GCGTGACCCGAGCCGCCGCGGTGGTGTTGAAGTGGA |
| attachment | | | | AATATCCGGCCGCCTCGCGGCGCTCTTGAATGCGCC |
| sites | | | | AGTGTACCCAGGCCATTTGCGTTCCCCTGTCGGTGG |
| | | | | GAACGCTGGTAGCGGGGGCTCGTCTCATTCAATCCC |
| | | | | ACCGACTGGCCAAGTGATACCCTTTAGGCTGACTTG |
| | | | | CCGGGGTCCGCTGGCGGCATGACCTACTGCCACTTC |
| | | | | TCCATGCGAGTAACTGCCGCCGCGGGCACCACGATG |
| | | | | GTCTTGCGGTCAAAGTAGAATACT |
| MG178 | 7325 | MG178- | nucleo- | GGCCATCCGCGAGCTAGTGGAGACGGTTACCGTCAG |
| recombinases | | 7185 AttR | tide | GCGTGACCCGAGCCGCCGCGGTGGTGTTGAAGTGGA |
| attachment | | | | AATATCCGGCCGCCTCGCGGCGCTCTTGAATGCGCC |
| sites | | | | AGTGTACCCAGGCCATTTGCGTTCCCCTGTCGGTGG |
| | | | | GAACGCTGGTAGCGGGGGAGGGACTCGAACCCCCG |
| | | | | ACACGCGGATTATGATTCCGCTGCTCTAACCAGCTG |
| | | | | AGCTACCCCGCCGCCACCAGCCCGGCGAAACCGGGC |
| | | | | ATTTCAACCAACCATATAAGGGGTTGGGCGCCGGTT |
| | | | | CTAAAGGCGTGCGAGCCGGTGTCAA |
| MG178 | 7326 | MG178- | nucleo- | TGGTAGCGGGGG |
| conserved | | 7185 Core | tide | |
| core | | | | |
| MG178 | 7327 | MG178- | nucleo- | ATGACGCCGTCGGCGAGCGCGTCGGGAGCCAGGTAC |
| recombinases | | 7186 AttB | tide | ACGTCGACGGTGCGACCGAGCGCGACATCGACCGTG |
| attachment | | | | CGGACGTCGCCGGCGGCGACCGCCTGGCCCGCCACG |
| sites | | | | ATCGTGCGGGCAGCGGCGAGCACCGGAACCGTCTGA |
| | | | | CGCGAGGCCGAGACGACGAACCACACGCCCGCGAT |
| | | | | CGACGCGACGACCAGCACCACGCCGATGAGGAATC |
| | | | | GGGCGTCGGACCAGAACGGCTTCGGGCGTGGTCGGG |
| | | | | AGGCGTCGATCGCGGTCATGCCAACCATCGTGACCC |
| | | | | AGGCCGCGGAGTGCCCCTC |
| MG178 | 7328 | MG178- | nucleo- | ATGACGCCGTCGGCGAGCGCGTCGGGAGCCAGGTAC |
| recombinases | | 7186 AttL | tide | ACGTCGACGGTGCGACCGAGCGCGACATCGACCGTG |
| attachment | | | | CGGACGTCGCCGGCGGCGACCGCCTGGCCCGCCACG |
| sites | | | | ATCGTGCGGGCAGCGGCGAGCACCGGAACCGTCTGA |
| | | | | CGCGAGGCCGACCCTATGGTGTAATTACCACCGTGA |
| | | | | GGGCAGCGACCTACGAGCGGATCTCCCAAGACCGG |
| | | | | GAATCGACCGAGCACGGCGTTGACAACCAACGGTCG |
| | | | | GCCAATCTCGCCTTGGCCGCAAGGCTCGGGTTCGAT |
| | | | | GTGGTCTCGGACTACCGC |
| MG178 | 7329 | MG178- | nucleo- | CGATCGTCTCCGCACTGGCCGCAAGCGTGCCCACTG |
| recombinases | | 7186 AttP | tide | TAATGGAGGACATCGCCCGACCGCCCAGCGCATACT |
| attachment | | | | TCGTACGTTGCGAGCATCTCCAGTGGCAGGAGCCGA |
| sites | | | | TTCACATCGAGTATCTGTAGCCGGGGTGCCTATTGC |
| | | | | GCCATTGCCGACCCTATGGTGTAATTACCACCGTGA |
| | | | | GGGCAGCGACCTACGAGCGGATCTCCCAAGACCGG |
| | | | | GAATCGACCGAGCACGGCGTTGACAACCAACGGTCG |
| | | | | GCCAATCTCGCCTTGGCCGCAAGGCTCGGGTTCGAT |
| | | | | GTGGTCTCGGACTACCGC |
| MG178 | 7330 | MG178- | nucleo- | CGATCGTCTCCGCACTGGCCGCAAGCGTGCCCACTG |
| recombinases | | 7186 AttR | tide | TAATGGAGGACATCGCCCGACCGCCCAGCGCATACT |
| attachment | | | | TCGTACGTTGCGAGCATCTCCAGTGGCAGGAGCCGA |
| sites | | | | TTCACATCGAGTATCTGTAGCCGGGGTGCCTATTGC |
| | | | | GCCATTGCCGAGACGACGAACCACACGCCCGCGATC |
| | | | | GACGCGACGACCAGCACCACGCCGATGAGGAATCG |
| | | | | GGCGTCGGACCAGAACGGCTTCGGGCGTGGTCGGGA |
| | | | | GGCGTCGATCGCGGTCATGCCAACCATCGTGACCCA |
| | | | | GGCCGCGGAGTGCCCCTC |
| MG178 | | MG178- | nucleo- | GCCGA |
| conserved | | 7186 Core | tide | |
| core | | | | |
| MG178 | 7332 | MG178- | nucleo- | CCAAGCGCCGACCCAGCATGCCGGAACTAGCCAATG |
| recombinases | | 7187 AttB | tide | GAATAATGCTATAGATCTAAAATGAGAAATAGCCAA |
| attachment | | | | AGTTTTGGGCTCGAACAAGAAATGGGATAATAGAAG |
| sites | | | | ATTTTGTATACAAAAAAAGCCCCAATTAGGGGCTAA |
| | | | | ATCAATAAATGGTGGACCGTCGGGGACTCGAACCCC |
| | | | | GGACCTTGGGATTAAGAGTCCCCTGCTCTACCAACT |
| | | | | AAGCTAACGGTCCATGAGAATGCCGTGTATTTCAAC |
| | | | | GGCTATCTATTATGCATCATTGATGCGAATTATCAAG |
| | | | | GTTGCGCTGGGGTGGGTAATC |
| MG178 | 7333 | MG178- | nucleo- | CCAAGCGCCGACCCAGCATGCCGGAACTAGCCAATG |
| recombinases | | 7187 AttL | tide | GAATAATGCTATAGATCTAAAATGAGAAATAGCCAA |
| attachment | | | | AGTTTTGGGCTCGAACAAGAAATGGGATAATAGAAG |
| sites | | | | ATTTTGTATACAAAAAAAGCCCCAATTAGGGGCTAA |
| | | | | ATCAATAAATGGTGGAGCTACGCACACTTAATCAGA |
| | | | | ACAACGTTATTTGCATCAATTCAACGAGGATGGAAA |
| | | | | TCTCAAAACATTGGTTTGCGGTGATAACGCTATTGG |
| | | | | ACGATAGGTTCGAAGATGGACGAAAGAAGCCCAAG |
| | | | | AAGGATAAAGACGCCTAGGCAGA |
| MG178 | 7334 | MG178- | nucleo- | GCGAACACCCAGATGAAAGGGTGTTCGCCTATAGAA |
| recombinases | | 7187 AttP | tide | TCAATGGTGGACCGTCGGGGACTCGAACCCCGGACC |
| attachment | | | | TTGGGATTAAGAGTCCCCTGCTCTACCAACTAAGCT |
| sites | | | | AACGGTCCATGGTAAACGCTCTAAACGAACGTGAAA |
| | | | | AATCTCAAATGGTGGAGCTACGCACACTTAATCAGA |
| | | | | ACAACGTTATTTGCATCAATTCAACGAGGATGGAAA |
| | | | | TCTCAAAACATTGGTTTGCGGTGATAACGCTATTGG |
| | | | | ACGATAGGTTCGAAGATGGACGAAAGAAGCCCAAG |
| | | | | AAGGATAAAGACGCCTAGGCAGA |
| MG178 | 7335 | MG178- | nucleo- | GCGAACACCCAGATGAAAGGGTGTTCGCCTATAGAA |
| recombinases | | 7187 AttR | tide | TCAATGGTGGACCGTCGGGGACTCGAACCCCGGACC |
| attachment | | | | TTGGGATTAAGAGTCCCCTGCTCTACCAACTAAGCT |
| sites | | | | AACGGTCCATGGTAAACGCTCTAAACGAACGTGAAA |
| | | | | AATCTCAAATGGTGGACCGTCGGGGACTCGAACCCC |
| | | | | GGACCTTGGGATTAAGAGTCCCCTGCTCTACCAACT |
| | | | | AAGCTAACGGTCCATGAGAATGCCGTGTATTTCAAC |
| | | | | GGCTATCTATTATGCATCATTGATGCGAATTATCAAG |
| | | | | GTTGCGCTGGGGTGGGTAATC |
| MG178 | 7336 | MG178- | nucleo- | AAATGGTGGA |
| conserved | | 7187 Core | tide | |
| core | | | | |
| MG178 | 7337 | MG178- | nucleo- | ATCGAGAGCTACCAGCCCGACAAGGGAACCAAGCT |
| recombinases | | 7188 AttB | tide | CGCCACCTTTGCGGCTCGTTGTATCGAAAACGAGAT |
| attachment | | | | TTTGATGCATCTCCGTTCCCTGAAAAAAACGCGCAA |
| sites | | | | GGATGTGTCCCTGCACGATCCGATCGGAACGGACAA |
| | | | | AGAGGGCAACGAGTTTACGTTAATCGATATCCTGGG |
| | | | | AACCGATACCGACGAAGTCGTCGACAAAGTGCAGCT |
| | | | | GAAAATCGAGAAAAGCAAAATTTTTCGCAACCTGGA |
| | | | | CATTCTCGATGAACGCGAAAAAGAAGTGGTGATCGG |
| | | | | CCGTTTCGGCCTCGATGCGGGCGG |
| MG178 | 7338 | MG178- | nucleo- | ATCGAGAGCTACCAGCCCGACAAGGGAACCAAGCT |
| recombinases | | 7188 AttL | tide | CGCCACCTTTGCGGCTCGTTGTATCGAAAACGAGAT |
| attachment | | | | TTTGATGCATCTCCGTTCCCTGAAAAAAACGCGCAA |
| sites | | | | GGATGTGTCCCTGCACGATCCGATCGGAACGGACAA |
| | | | | AGAGGGCAACGAGTTTACATCAAATGGTTATCTGTA |
| | | | | TCCTTATGTACATCCTACCCATTATATGGTATAATAC |
| | | | | CCTTAACGATGCGGTGGCGGAATAGGTAGACGCACA |
| | | | | ACCTCAAGGGCAATAACGGGCGCGGTAAGGGTGCA |
| | | | | GCCCACTAAGGACCGCCCGCGTCA |
| MG178 | 7339 | MG178- | nucleo- | TGGAAATCCAAGTACCCCGCAAAGAAAAACGCCCTT |
| recombinases | | 7188 AttP | tide | CTCAAATCCGTCCTCCTTCATGCCACTTACAAAAAA |
| attachment | | | | GAAAAGTGGCAGCGTAAAGATCAATTCGAACTTGTC |
| sites | | | | CTGGTGCCGAAGTTCAAATAATACAGATAAGGTATT |
| | | | | GATGAGAACGAGTTTACATCAAATGGTTATCTGTAT |
| | | | | CCTTATGTACATCCTACCCATTATATGGTATAATACC |
| | | | | CTTAACGATGCGGTGGCGGAATAGGTAGACGCACAA |
| | | | | CCTCAAGGGCAATAACGGGCGCGGTAAGGGTGCAG |
| | | | | CCCACTAAGGACCGCCCGCGTCA |
| MG178 | 7340 | MG178- | nucleo- | TGGAAATCCAAGTACCCCGCAAAGAAAAACGCCCTT |
| recombinases | | 7188 AttR | tide | CTCAAATCCGTCCTCCTTCATGCCACTTACAAAAAA |
| attachment | | | | GAAAAGTGGCAGCGTAAAGATCAATTCGAACTTGTC |
| sites | | | | CTGGTGCCGAAGTTCAAATAATACAGATAAGGTATT |
| | | | | GATGAGAACGAAATTACGTTAATCGATATCCTGGGA |
| | | | | ACCGATACCGACGAAGTCGTCGACAAAGTGCAGCTG |
| | | | | AAAATCGAGAAAAGCAAAATTTTTCGCAACCTGGAC |
| | | | | ATTCTCGATGAACGCGAAAAAGAAGTGGTGATCGGC |
| | | | | CGTTTCGGCCTCGATGCGGGCGG |
| MG178 | 7341 | MG178- | nucleo- | AACGAAATTAC |
| conserved | | 7188 Core | tide | |
| core | | | | |
| MG178 | 7342 | MG178- | nucleo- | ACGCAAAGTTGTCTGCTGTTCAATGTCCTAATTGCAA |
| recombinases | | 7189 AttB | tide | AAAAAATCTTCGAGATCACCTCGAAGATTTTTTTATC |
| attachment | | | | TCATTGTATACCCGAAAACTATTTCACAAAAAAATA |
| sites | | | | AGTTCGGATAATACTCTTTTGGTGGAGAATTGCGCC |
| | | | | CGTCACTCGAACTCACTACCTCTACAATGCGAATGT |
| | | | | AGCGCTCTCCCAGATGAGCTACGCCCCCAAGCGCTT |
| | | | | ATAAAGTATAGCAGATTTTTTGTCGTTTGTCAATACC |
| | | | | GCCGCAAAATAAATTTGAAAAGGAAACGCAAATTTT |
| | | | | CGTGACGGTGTATTCGGTG |
| MG178 | 7343 | MG178- | nucleo- | ACGCAAAGTTGTCTGCTGTTCAATGTCCTAATTGCAA |
| recombinases | | 7189 AttL | tide | AAAAAATCTTCGAGATCACCTCGAAGATTTTTTTATC |
| attachment | | | | TCATTGTATACCCGAAAACTATTTCACAAAAAAATA |
| sites | | | | AGTTCGGATAATACTCTTTTGGTGGAGAATTGCGCC |
| | | | | CGTCACTCGAACTCTTTATAGTCTACAAGAATGTATT |
| | | | | CTTAACCACGTCCGCATTGCGATGATTTTATTTTTTC |
| | | | | AAAAACGCTTCGACCGCTTCTCTTACGATCTGCGCCT |
| | | | | GAGAAATACCCTCGCGGACGCACTTTGTCTTAAACT |
| | | | | CTTCGACCATTGCCTTT |
| MG178 | 7344 | MG178- | nucleo- | TTTCTCAATGCCGTTTTTGTCTATGACGAAAGTGTTA |
| recombinases | | 7189 AttP | tide | CATTTGCATTCAATTACTCCAATAACGGAGAAAAAG |
| attachment | | | | TCACCCTCTCCGAAGTTGATAATATCAACGGTTCAG |
| sites | | | | GTGACTTTTTCGAGTGTGGTTACGATGGTGGAGACG |
| | | | | ATGAGACTCGAACTCTTTATAGTCTACAAGAATGTA |
| | | | | TTCTTAACCACGTCCGCATTGCGATGATTTTATTTTT |
| | | | | TCAAAAACGCTTCGACCGCTTCTCTTACGATCTGCGC |
| | | | | CTGAGAAATACCCTCGCGGACGCACTTTGTCTTAAA |
| | | | | CTCTTCGACCATTGCCTTT |
| MG178 | 7345 | MG178- | nucleo- | TTTCTCAATGCCGTTTTTGTCTATGACGAAAGTGTTA |
| recombinases | | 7189 AttR | tide | CATTTGCATTCAATTACTCCAATAACGGAGAAAAAG |
| attachment | | | | TCACCCTCTCCGAAGTTGATAATATCAACGGTTCAG |
| sites | | | | GTGACTTTTTCGAGTGTGGTTACGATGGTGGAGACG |
| | | | | ATGAGACTCGAACTCACTACCTCTACAATGCGAATG |
| | | | | TAGCGCTCTCCCAGATGAGCTACGCCCCCAAGCGCT |
| | | | | TATAAAGTATAGCAGATTTTTTGTCGTTTGTCAATAC |
| | | | | CGCCGCAAAATAAATTTGAAAAGGAAACGCAAATTT |
| | | | | TCGTGACGGTGTATTCGGTG |
| MG178 | 7346 | MG178- | nucleo- | ACTCGAACTC |
| conserved | | 7189 Core | tide | |
| core | | | | |
| MG178 | 7347 | MG178- | nucleo- | AAGTTCCCTCTCAAAAAAATCCTTAATCAGCTTTATA |
| recombinases | | 7190 AttB | tide | GCAATTTCTGTTTCCATTATATTCTGTCTGGAAGTGT |
| attachment | | | | ATCCTTCCGGTATTATAATACTTCCCATGTTTCTACT |
| sites | | | | CCTAACTACAAATCTACAAAAAAAAATTACTAATTA |
| | | | | AAATGGCGCACCCAGCAGGAGTTGAACCCACAACCT |
| | | | | TCTGATCCGTAGTCAGACGCTCTATCCAATTGAGCTA |
| | | | | TGGATGCACATTTAAGTATATATTTTAAAAAAAAAT |
| | | | | GGCGGAGAAGGAGGGATTTGAACCCTCGATCCAAGT |
| | | | | TTTAGCCCGGATACTCCCT |
| MG178 | 7348 | MG178- | nucleo- | AAGTTCCCTCTCAAAAAAATCCTTAATCAGCTTTATA |
| recombinases | | 7190 AttL | tide | GCAATTTCTGTTTCCATTATATTCTGTCTGGAAGTGT |
| attachment | | | | ATCCTTCCGGTATTATAATACTTCCCATGTTTCTACT |
| sites | | | | CCTAACTACAAATCTACAAAAAAAAATTACTAATTA |
| | | | | AAATGGCGCACCCAAGTGCATACTTAGTCACCACAA |
| | | | | CTAAGAATCCTTGAATTGCAATATTTTATTTTTTCAT |
| | | | | GTTCCTTATAATACCATAATTTACTTGAATTTGCAAT |
| | | | | TCTTTTCATTGAGTTCCTCTTCTGAAATGCGATGCAC |
| | | | | TAAGCCTTTCAACTTTT |
| MG178 | 7349 | MG178- | nucleo- | GTATCAGAATTAAAAGAAATTTTGAATCTGATTATA |
| recombinases | | 7190 AttP | tide | CAAAAAATTGTTCTAAGTAAAAATGGAGAAATAGA |
| attachment | | | | AGTAATATTTTAAAAGAAAAAGACCCAATAAAAATT |
| sites | | | | TTAGGTCTTTTTTTATTTATTATAAAAAATTGTGTTTT |
| | | | | TTAACTGGCGCACCCAAGTGCATACTTAGTCACCAC |
| | | | | AACTAAGAATCCTTGAATTGCAATATTTTATTTTTTC |
| | | | | ATGTTCCTTATAATACCATAATTTACTTGAATTTGCA |
| | | | | ATTCTTTTCATTGAGTTCCTCTTCTGAAATGCGATGC |
| | | | | ACTAAGCCTTTCAACTTTT |
| MG178 | 7350 | MG178- | nucleo- | GTATCAGAATTAAAAGAAATTTTGAATCTGATTATA |
| recombinases | | 7190 AttR | tide | CAAAAAATTGTTCTAAGTAAAAATGGAGAAATAGA |
| attachment | | | | AGTAATATTTTAAAAGAAAAAGACCCAATAAAAATT |
| sites | | | | TTAGGTCTTTTTTTATTTATTATAAAAAATTGTGTTTT |
| | | | | TTAACTGGCGCACCCAGCAGGAGTTGAACCCACAAC |
| | | | | CTTCTGATCCGTAGTCAGACGCTCTATCCAATTGAGC |
| | | | | TATGGATGCACATTTAAGTATATATTTTAAAAAAAA |
| | | | | ATGGCGGAGAAGGAGGGATTTGAACCCTCGATCCAA |
| | | | | GTTTTAGCCCGGATACTCCCT |
| MG178 | 7351 | MG178- | nucleo- | TGGCGCACCCA |
| conserved | | 7190 Core | tide | |
| core | | | | |
| MG178 | 7352 | MG178- | nucleo- | GATCGAGATTCCAACGGTACTGCCCCTGCCGATCCC |
| recombinases | | 7191 AttB | tide | GGAAGTGTCCGTAAATACAAGAAAAGGCGTAGTCGT |
| attachment | | | | CAACTACGCCCCCTGTGAACTCGCTGTATAAAAAGC |
| sites | | | | GAGTGTTCTTACAGCCTTTCAAATGCTATAAGAACA |
| | | | | CTCGATATGGTTGCGGAGACAGGATTTGAACCTGCG |
| | | | | ACCTCCGGGTTATGAGCCCGACGAGCTACCGAACTG |
| | | | | CTCCACTCCGCGATATGAACTTGACCCCTCAAGGCT |
| | | | | CATATACTATAACATATGAGCCTTGGGTTGTCAAGT |
| | | | | GTTATTTTAGCGCTCGGAAACAGAAGA |
| MG178 | 7353 | MG178- | nucleo- | GATCGAGATTCCAACGGTACTGCCCCTGCCGATCCC |
| recombinases | | 7191 AttL | tide | GGAAGTGTCCGTAAATACAAGAAAAGGCGTAGTCGT |
| attachment | | | | CAACTACGCCCCCTGTGAACTCGCTGTATAAAAAGC |
| sites | | | | GAGTGTTCTTACAGCCTTTCAAATGCTATAAGAACA |
| | | | | CTCGATATGGTTGCGGAGACACACAACATAGATTAC |
| | | | | CAGCGATTTTTTCCGGAAATCCTTTTTATGTTCACAT |
| | | | | CAAGCATTTAAGTGCTGTATTTCACGCTGTTCTGCTG |
| | | | | TCTAATGTGGCTACGATTTGCGCCTTTTCTATAGAGT |
| | | | | ATCCTAGTCGGTGAAGCAAGGACA |
| MG178 | 7354 | MG178- | nucleo- | GTTGATACAATCACGCGGTGGTTGAATGCCTTGAAA |
| recombinases | | 7191 AttP | tide | AACAATCCGGATGAAAAGGCCGTAAAGCTGCTTGTG |
| attachment | | | | AAGCGAATTGACGTTTCCGGAGATAAAAAGAACAA |
| sites | | | | CGTGTTCAATATACAAAGCACATTGAACACGTTGTT |
| | | | | GGAAATAATGGTTGCGGAGACACACAACATAGATTA |
| | | | | CCAGCGATTTTTTCCGGAAATCCTTTTTATGTTCACA |
| | | | | TCAAGCATTTAAGTGCTGTATTTCACGCTGTTCTGCT |
| | | | | GTCTAATGTGGCTACGATTTGCGCCTTTTCTATAGAG |
| | | | | TATCCTAGTCGGTGAAGCAAGGACA |
| MG178 | 7355 | MG178- | nucleo- | GTTGATACAATCACGCGGTGGTTGAATGCCTTGAAA |
| recombinases | | 7191 AttR | tide | AACAATCCGGATGAAAAGGCCGTAAAGCTGCTTGTG |
| attachment | | | | AAGCGAATTGACGTTTCCGGAGATAAAAAGAACAA |
| sites | | | | CGTGTTCAATATACAAAGCACATTGAACACGTTGTT |
| | | | | GGAAATAATGGTTGCGGAGACAGGATTTGAACCTGC |
| | | | | GACCTCCGGGTTATGAGCCCGACGAGCTACCGAACT |
| | | | | GCTCCACTCCGCGATATGAACTTGACCCCTCAAGGC |
| | | | | TCATATACTATAACATATGAGCCTTGGGTTGTCAAGT |
| | | | | GTTATTTTAGCGCTCGGAAACAGAAGA |
| MG178 | 7356 | MG178- | nucleo- | ATGGTTGCGGAGACA |
| conserved | | 7191 Core | tide | |
| core | | | | |
| MG178 | 7357 | MG178- | nucleo- | GTTTTACCCGAATTTTTTTGAAAAAAGGTCTTTACAA |
| recombinases | | 7192 AttB | tide | ACTCCCCATGATATGCTATTATAGTCAAGCAGTCCA |
| attachment | | | | AAAACACATGGGGGCGTAGCTCACTTGGGAGAGCG |
| sites | | | | CTTGACTGGCAGTCAAGAGGTAGAGAGTTCGATCCT |
| | | | | CTTCGTCTCCACCAAAAGAGATCCGCAGGAACACAG |
| | | | | TTTCTGCGGTCTTTTTGTATAATCGACCGACTGTTTTT |
| | | | | GTTCCCGTTCCTCATCTACAAAGGAGACACCATGAA |
| | | | | AAAGAAACTGACCGTAAAAGAGTACGTCTATGTTGC |
| | | | | CAGCATGCTGTTTGGCTTGT |
| MG178 | 7358 | MG178- | nucleo- | GTTTTACCCGAATTTTTTTGAAAAAAGGTCTTTACAA |
| recombinases | | 7192 AttL | tide | ACTCCCCATGATATGCTATTATAGTCAAGCAGTCCA |
| attachment | | | | AAAACACATGGGGGCGTAGCTCACTTGGGAGAGCG |
| sites | | | | CTTGACTGGCAGTCAAGAGGTAGAGAGTTCGATCCT |
| | | | | CTTCGTCTCCACCAAATAGTGCAAATCCGAACTCTGT |
| | | | | GTTCTTCATCAAACACACCTTTGGATTTGTTTACAAG |
| | | | | ATAGAGAACGCTGATTGAATCGGCGTTCTTTTTCTTT |
| | | | | GCCACGGGACGAGATGAAGTAGTTAAAGTAGTTGTT |
| | | | | TTTCGGTTTTTGCGTAAAC |
| MG178 | 7359 | MG178- | nucleo- | ATGAGGATTACCAACGGCGCGTGATTGATACATTGG |
| recombinases | | 7192 AttP | tide | TAAACTCTGTATATGTGTATGACGATGAAGATGGTG |
| attachment | | | | GGAAGCGGATTATGCTAACATTCAATCTTTCGGGCA |
| sites | | | | ATAATACCGCTACTCTCACGAGTTCGGATATTGGGT |
| | | | | GTTATGCTCCACCAAATAGTGCAAATCCGAACTCTG |
| | | | | TGTTCTTCATCAAACACACCTTTGGATTTGTTTACAA |
| | | | | GATAGAGAACGCTGATTGAATCGGCGTTCTTTTTCTT |
| | | | | TGCCACGGGACGAGATGAAGTAGTTAAAGTAGTTGT |
| | | | | TTTTCGGTTTTTGCGTAAAC |
| MG178 | 7360 | MG178- | nucleo- | ATGAGGATTACCAACGGCGCGTGATTGATACATTGG |
| recombinases | | 7192 AttR | tide | TAAACTCTGTATATGTGTATGACGATGAAGATGGTG |
| attachment | | | | GGAAGCGGATTATGCTAACATTCAATCTTTCGGGCA |
| sites | | | | ATAATACCGCTACTCTCACGAGTTCGGATATTGGGT |
| | | | | GTTATGCTCCACCAAAAGAGATCCGCAGGAACACAG |
| | | | | TTTCTGCGGTCTTTTTGTATAATCGACCGACTGTTTTT |
| | | | | GTTCCCGTTCCTCATCTACAAAGGAGACACCATGAA |
| | | | | AAAGAAACTGACCGTAAAAGAGTACGTCTATGTTGC |
| | | | | CAGCATGCTGTTTGGCTTGT |
| MG178 | 7361 | MG178- | nucleo- | CTCCACCAAA |
| conserved | | 7192 Core | tide | |
| core | | | | |
| MG178 | 7362 | MG178- | nucleo- | CATTCCCATCAATACCATCGTCTCCAGCCTGGAAAG |
| recombinases | | 7194 AttB | tide | CATATCTGCCGTACTGTTGGCATGGCCGGTACTAAG |
| attachment | | | | ACTGCCATCGTGTCCAGTATTTAGGGCCTGCAGCAT |
| sites | | | | ATCGATCGCCTCCGCTCCCCGGACCTCGCCCACAAT |
| | | | | AATCCGATCTGGGCGCATTCTCAGGGCCGACTTGAT |
| | | | | CAGATCGCGGATCGTCACGGCGCCTGTCCCCTCCAC |
| | | | | ATTGGGATTCCTTGCCTCAAGGCTCACCAAATTGGG |
| | | | | GATTTCCTGCAATTTTAATTCTGCGTTGTCTTCAATG |
| | | | | GTTATAATTCTTTCATCCTTTGG |
| MG178 | 7363 | MG178- | nucleo- | CATTCCCATCAATACCATCGTCTCCAGCCTGGAAAG |
| recombinases | | 7194 AttL | tide | CATATCTGCCGTACTGTTGGCATGGCCGGTACTAAG |
| attachment | | | | ACTGCCATCGTGTCCAGTATTTAGGGCCTGCAGCAT |
| sites | | | | ATCGATCGCCTCCGCTCCCCGGACCTCGCCCACAAT |
| | | | | AATCCGATCTGGGCGCATGATAAAAATAAGAGATTC |
| | | | | CTCCTCCAGTCGCTTCCATATATTACCGTTTGAAATT |
| | | | | ATATATCACTTTGTATACAATATTTATGGAGGAACA |
| | | | | ATTACAATGAACCAGTTTGCAAAACGCTTAAAATAC |
| | | | | TTAAGAATAGAAAGAAATCTTAC |
| MG178 | 7364 | MG178- | nucleo- | AACAAAGAAAAATTACCGACTTTATTCGTAATTTTG |
| recombinases | | 7194 AttP | tide | AAAACTTTACTCCTGAAGAAAGAAACGCCATAGCCC |
| attachment | | | | GTACATGTATAAAAGAATGCGTTTGGGATGGTCATA |
| sites | | | | CGCTTTCTGTCGTCCTGTAGTTCTCTTTATTATCATGC |
| | | | | GTCTATCTGGGCGCATGATAAAAATAAGAGATTCCT |
| | | | | CCTCCAGTCGCTTCCATATATTACCGTTTGAAATTAT |
| | | | | ATATCACTTTGTATACAATATTTATGGAGGAACAATT |
| | | | | ACAATGAACCAGTTTGCAAAACGCTTAAAATACTTA |
| | | | | AGAATAGAAAGAAATCTTAC |
| MG178 | 7365 | MG178- | nucleo- | AACAAAGAAAAATTACCGACTTTATTCGTAATTTTG |
| recombinases | | 7194 AttR | tide | AAAACTTTACTCCTGAAGAAAGAAACGCCATAGCCC |
| attachment | | | | GTACATGTATAAAAGAATGCGTTTGGGATGGTCATA |
| sites | | | | CGCTTTCTGTCGTCCTGTAGTTCTCTTTATTATCATGC |
| | | | | GTCTATCTGGGCGCATTCTCAGGGCCGACTTGATCA |
| | | | | GATCGCGGATCGTCACGGCGCCTGTCCCCTCCACAT |
| | | | | TGGGATTCCTTGCCTCAAGGCTCACCAAATTGGGGA |
| | | | | TTTCCTGCAATTTTAATTCTGCGTTGTCTTCAATGGTT |
| | | | | ATAATTCTTTCATCCTTTGG |
| MG178 | 7366 | MG178- | nucleo- | ATCTGGGCGCAT |
| conserved | | 7194 Core | tide | |
| core | | | | |
| MG178 | 7367 | MG178- | nucleo- | AGCCGATATCTTTTTGCTCCGCCTGGAGCACGAAGC |
| recombinases | | 7195 AttB | tide | ACCTGTGTTTTTGCACAGGTGCTTTTGTATTTTGGGC |
| attachment | | | | CTGGTCCTTATATTTATAGTTTAAAACCAAAATATTA |
| sites | | | | AAACCCCATAACTTCGCTGGAAGTTATGGGGTTTAC |
| | | | | TCTTCAATTTGGTGGAACTAACTGGACTCGAACCAG |
| | | | | TGACCCCCTCGATGTCAACGAGGTACTCTAACCAAC |
| | | | | TGAGCTATAGTTCCGCAACGATGTTATTTTAGCGTAG |
| | | | | AATCGTCATTTTGTCAAGGACGTTTTACTCCCTGACC |
| | | | | GTTACTGCGGTACCTACTTTG |
| MG178 | 7368 | MG178- | nucleo- | AGCCGATATCTTTTTGCTCCGCCTGGAGCACGAAGC |
| recombinases | | 7195 AttL | tide | ACCTGTGTTTTTGCACAGGTGCTTTTGTATTTTGGGC |
| attachment | | | | CTGGTCCTTATATTTATAGTTTAAAACCAAAATATTA |
| sites | | | | AAACCCCATAACTTCGCTGGAAGTTATGGGGTTTAC |
| | | | | TCTTCAATTTGGTGGAAGAATTTGTATCGTACCCAAA |
| | | | | CAAATATTTCACTATATAGAACGGCATTGATAATAC |
| | | | | GCATCGATGCCGCCTAAAAACTAGCCTTATAAAGTT |
| | | | | TTCTTATTAAACTTCAAGTCTTCTAATGTGCCGGTCT |
| | | | | TAAATTTGCTTTTCTTAAAAC |
| MG178 | 7369 | MG178- | nucleo- | ATGAGATCCGGCAGGAATTTTTAAATACTTTCGTATC |
| recombinases | | 7195 AttP | tide | TAAAGCGTATGTATTTTCAGATCATCTTTTTGTGATC |
| attachment | | | | TATGATGCCATTAACGGCATCAATACTGAAGTTACA |
| sites | | | | CCCGAAATTTTATCAAATCCAAACGAGTTCGGATAC |
| | | | | ATTCCAATTTGGTGGAAGAATTTGTATCGTACCCAA |
| | | | | ACAAATATTTCACTATATAGAACGGCATTGATAATA |
| | | | | CGCATCGATGCCGCCTAAAAACTAGCCTTATAAAGT |
| | | | | TTTCTTATTAAACTTCAAGTCTTCTAATGTGCCGGTC |
| | | | | TTAAATTTGCTTTTCTTAAAAC |
| MG178 | 7370 | MG178- | nucleo- | ATGAGATCCGGCAGGAATTTTTAAATACTTTCGTATC |
| recombinases | | 7195 AttR | tide | TAAAGCGTATGTATTTTCAGATCATCTTTTTGTGATC |
| attachment | | | | TATGATGCCATTAACGGCATCAATACTGAAGTTACA |
| sites | | | | CCCGAAATTTTATCAAATCCAAACGAGTTCGGATAC |
| | | | | ATTCCAATTTGGTGGAACTAACTGGACTCGAACCAG |
| | | | | TGACCCCCTCGATGTCAACGAGGTACTCTAACCAAC |
| | | | | TGAGCTATAGTTCCGCAACGATGTTATTTTAGCGTAG |
| | | | | AATCGTCATTTTGTCAAGGACGTTTTACTCCCTGACC |
| | | | | GTTACTGCGGTACCTACTTTG |
| MG178 | 7371 | MG178- | nucleo- | CAATTTGGTGGAA |
| conserved | | 7195 Core | tide | |
| core | | | | |
| MG178 | 7372 | MG178- | nucleo- | CTTCGGCAGGATAAAAAAAGAGTCCGAACGCGAATT |
| recombinases | | 7196 AttB | tide | GCGTCCAGACTCAGCCAAGGAGAAGGAAAGATTCA |
| attachment | | | | CTGTACCTGCCGTCGCGAGCCGCTTCAGCAGGATAA |
| sites | | | | AAGAAAAGAGTCCGAACGCGAATTGCGTCCAGACTC |
| | | | | AGTCTGGTGGTTGCGGGAGCTGGATTTGAACCAACG |
| | | | | ACCTTCGGGTTATGAGCCCGACGAGCTACCAAACTG |
| | | | | CTCCATCCCGCGATATTGAATTTCCAGTGCTCTACTA |
| | | | | TAATACCACATTTCGGAGAAAAATGCAAGTCTTTTTT |
| | | | | TGATTTTTTTCAAAAGAGTGGGA |
| MG178 | 7373 | MG178- | nucleo- | CTTCGGCAGGATAAAAAAAGAGTCCGAACGCGAATT |
| recombinases | | 7196 AttL | tide | GCGTCCAGACTCAGCCAAGGAGAAGGAAAGATTCA |
| attachment | | | | CTGTACCTGCCGTCGCGAGCCGCTTCAGCAGGATAA |
| sites | | | | AAGAAAAGAGTCCGAACGCGAATTGCGTCCAGACTC |
| | | | | AGTCTGGTGGTTGCGGGAGTTCGCAACATCTTTTACC |
| | | | | AAGAATTTTATTGAGATTTATCTAATATAATATCATA |
| | | | | TATTGATTAATCAATGAGATTAACAGTAAAAAGTGA |
| | | | | GGAAGCAAGCTTTTAAGCTTTATTTCTTATCATTCTG |
| | | | | GCGTGAATCGAGATAAGATTCC |
| MG178 | 7374 | MG178- | nucleo- | CTTTACAGTCGATCAAATCCGTGCATGGTTGGAAGC |
| recombinases | | 7196 AttP | tide | ACTGAAAGCAACTCCCGATGATAAGGCAGTCCGTTT |
| attachment | | | | GCTTATTTCTCGCATTGACATAAAACAAAAGACCAT |
| sites | | | | TATTAACATGGAAAGCACGTTAACAATGGTCTTAAG |
| | | | | TGAAATTGGTTGCGGGAGTTCGCAACATCTTTTACC |
| | | | | AAGAATTTTATTGAGATTTATCTAATATAATATCATA |
| | | | | TATTGATTAATCAATGAGATTAACAGTAAAAAGTGA |
| | | | | GGAAGCAAGCTTTTAAGCTTTATTTCTTATCATTCTG |
| | | | | GCGTGAATCGAGATAAGATTCC |
| MG178 | 7375 | MG178- | nucleo- | CTTTACAGTCGATCAAATCCGTGCATGGTTGGAAGC |
| recombinases | | 7196 AttR | tide | ACTGAAAGCAACTCCCGATGATAAGGCAGTCCGTTT |
| attachment | | | | GCTTATTTCTCGCATTGACATAAAACAAAAGACCAT |
| sites | | | | TATTAACATGGAAAGCACGTTAACAATGGTCTTAAG |
| | | | | TGAAATTGGTTGCGGGAGCTGGATTTGAACCAACGA |
| | | | | CCTTCGGGTTATGAGCCCGACGAGCTACCAAACTGC |
| | | | | TCCATCCCGCGATATTGAATTTCCAGTGCTCTACTAT |
| | | | | AATACCACATTTCGGAGAAAAATGCAAGTCTTTTTTT |
| | | | | GATTTTTTTCAAAAGAGTGGGA |
| MG178 | 7376 | MG178- | nucleo- | TGGTTGCGGGAG |
| conserved | | 7196 Core | tide | |
| core | | | | |
| MG178 | 7377 | MG178- | nucleo- | TTAAGAAAGCTTAAGAGGCCAGAAGCTCCTTACAGT |
| recombinases | | 7197 AttB | tide | TGGACCCATCTGAACCGAATTTTGCTAAAAGCAGAC |
| attachment | | | | GAAAAAACGGGAACGCCGTTCTTTTGCTCCCTAAAA |
| sites | | | | GATCAAGCCTCCTGTCCATGGACAGGAGGCTTGCAA |
| | | | | GTTATACAGTTGGTGGGCCCACTAGGATTCGAACCT |
| | | | | AGGACCAACCGGTTATGAGCCGGGGGCTCTACCGCT |
| | | | | GAGCTATAGGCCCAATGTAGGAATTATACCCCAGAA |
| | | | | AAAGATGCGCTGTCAACGTTTCTTACTCGAGGAATT |
| | | | | CCTTGAGGGGTTTGCTCCGTTTCG |
| MG178 | 7378 | MG178- | nucleo- | TTAAGAAAGCTTAAGAGGCCAGAAGCTCCTTACAGT |
| recombinases | | 7197 AttL | tide | TGGACCCATCTGAACCGAATTTTGCTAAAAGCAGAC |
| attachment | | | | GAAAAAACGGGAACGCCGTTCTTTTGCTCCCTAAAA |
| sites | | | | GATCAAGCCTCCTGTCCATGGACAGGAGGCTTGCAA |
| | | | | GTTATACAGTTGGTGGGCTATCTAACCCTTAGTGCG |
| | | | | AACCTAAAAGTACGGATAGGGCGTGGCTGGTTTGCC |
| | | | | TTATCCTATCCAATCCCAGTCTAGCGATATTGTCAAC |
| | | | | TTTTATGAAAAATAAATCACCCCGTAAAGCCAGTTA |
| | | | | ATGCCTGACTCTACGGGGATTCT |
| MG178 | 7379 | MG178- | nucleo- | CAGGAATACAGAACCAAGCTGCTAGATATCTTGGTC |
| recombinases | | 7197 AttP | tide | AGCCGAGTTGTTCTCTACCCAAATAAAGCAGAGGTT |
| attachment | | | | TTTTATCGCTATCAAAAAGAACTCCCTTCCCTCCCTA |
| sites | | | | ATCCAGTGATTATCAGGGAAGAAAGGGGTTCGAATG |
| | | | | GCAATCAGTTGGTGGGCTATCTAACCCTTAGTGCGA |
| | | | | ACCTAAAAGTACGGATAGGGCGTGGCTGGTTTGCCT |
| | | | | TATCCTATCCAATCCCAGTCTAGCGATATTGTCAACT |
| | | | | TTTATGAAAAATAAATCACCCCGTAAAGCCAGTTAA |
| | | | | TGCCTGACTCTACGGGGATTCT |
| MG178 | 7380 | MG178- | nucleo- | CAGGAATACAGAACCAAGCTGCTAGATATCTTGGTC |
| recombinases | | 7197 AttR | tide | AGCCGAGTTGTTCTCTACCCAAATAAAGCAGAGGTT |
| attachment | | | | TTTTATCGCTATCAAAAAGAACTCCCTTCCCTCCCTA |
| sites | | | | ATCCAGTGATTATCAGGGAAGAAAGGGGTTCGAATG |
| | | | | GCAATCAGTTGGTGGGCCCACTAGGATTCGAACCTA |
| | | | | GGACCAACCGGTTATGAGCCGGGGGCTCTACCGCTG |
| | | | | AGCTATAGGCCCAATGTAGGAATTATACCCCAGAAA |
| | | | | AAGATGCGCTGTCAACGTTTCTTACTCGAGGAATTC |
| | | | | CTTGAGGGGTTTGCTCCGTTTCG |
| MG178 | 7381 | MG178- | nucleo- | CAGTTGGTGGGC |
| conserved | | 7197 Core | tide | |
| core | | | | |
| MG178 | 7382 | MG178- | nucleo- | GCCATCTGGTTCCTCAACTTCTCGACGTGGACGGGC |
| recombinases | | 6304 AttB | tide | CACCACGGCATCTACCTGGAGGACCTCTACGTCCGC |
| attachment | | | | CCCGAGGCGCGCGGCCTCGGGACCGGCCGGGCGCTC |
| sites | | | | CTCGCTGCCCTGGCCACCGTTGCCCACCGCTCCGACT |
| | | | | ACACCCGTATCGACTGGTCGGTGCTCGATTGGAACG |
| | | | | AGCCCGCGCTGCGCTTCTACCGGTCGCTGGGGGCCG |
| | | | | AGCCCATGGACGAATGGACCGGCTACCGGCTCTCGG |
| | | | | GCCCGGAGCTGGCCGCCCTGGCCGGCGGCGAACCCG |
| | | | | CCACGTGACCGGTCGGGCCCGGCCGG |
| MG178 | 7383 | MG178- | nucleo- | GCCATCTGGTTCCTCAACTTCTCGACGTGGACGGGC |
| recombinases | | 6304 AttL | tide | CACCACGGCATCTACCTGGAGGACCTCTACGTCCGC |
| attachment | | | | CCCGAGGCGCGCGGCCTCGGGACCGGCCGGGCGCTC |
| sites | | | | CTCGCTGCCCTGGCCACCGTTGCCCACCGCTCCGACT |
| | | | | ACACCCGTATCGACTGGTCGACTCTTGCGTTTGGTAA |
| | | | | CCAGAGAGAGCTACCCTGGTGGCATGACCCAACAAC |
| | | | | TGCGAGCTGCGATTTACTGCCGGATCTCCAAGGCTA |
| | | | | AGGGGACCAAGAAGACTCAGAGCGTCGAAGACCAG |
| | | | | GAGCGAGACTGCCGAGACCTCTGCGA |
| MG178 | 7384 | MG178- | nucleo- | TCAGCGGCTTCGAGTTCTTCTACAGCTCACTGTCGAA |
| recombinases | | 6304 AttP | tide | CATCCCCTACTATCGGAGGCACCCATCGGCCAGACT |
| attachment | | | | CGCTAGAACCACGAAATTCCCCTATGAATCGCGGCA |
| sites | | | | GAGCAGCCTGAGCTGGGCAAATGCTCGACTTATCAG |
| | | | | ATACACGTATCGACTGGTCGACTCTTGCGTTTGGTAA |
| | | | | CCAGAGAGAGCTACCCTGGTGGCATGACCCAACAAC |
| | | | | TGCGAGCTGCGATTTACTGCCGGATCTCCAAGGCTA |
| | | | | AGGGGACCAAGAAGACTCAGAGCGTCGAAGACCAG |
| | | | | GAGCGAGACTGCCGAGACCTCTGCGA |
| MG178 | 7385 | MG178- | nucleo- | TCAGCGGCTTCGAGTTCTTCTACAGCTCACTGTCGAA |
| recombinases | | 6304 AttR | tide | CATCCCCTACTATCGGAGGCACCCATCGGCCAGACT |
| attachment | | | | CGCTAGAACCACGAAATTCCCCTATGAATCGCGGCA |
| sites | | | | GAGCAGCCTGAGCTGGGCAAATGCTCGACTTATCAG |
| | | | | ATACACGTATCGACTGGTCGGTGCTCGATTGGAACG |
| | | | | AGCCCGCGCTGCGCTTCTACCGGTCGCTGGGGGCCG |
| | | | | AGCCCATGGACGAATGGACCGGCTACCGGCTCTCGG |
| | | | | GCCCGGAGCTGGCCGCCCTGGCCGGCGGCGAACCCG |
| | | | | CCACGTGACCGGTCGGGCCCGGCCGG |
| MG178 | 7386 | MG178- | nucleo- | CGTATCGACTGGTCG |
| conserved | | 6304 Core | tide | |
| core | | | | |
| MG178 | 7387 | MG178- | nucleo- | CAGCGAGCCGACCCCGTACAGGCTTAGAGCGCCGCC |
| recombinases | | 7199 AttB | tide | GGCATGGACCGGGCTGAAGCCGAGCTTCCGGGTCAG |
| attachment | | | | GTAGAGGGTCAGGAAAAAGATGACCATCGAGCCCG |
| sites | | | | AGGAATTGACCAGGTTGACCGTGAACAGGATCCAGG |
| | | | | CCTTGCGCGGCAGGCCGCTATACGCCTGGCGGTAGG |
| | | | | TGTCCCTGATCTTCCCGAGCATGGCCGTTAAAAGCG |
| | | | | TATTACACCAGGACGGGTGGGAACGTCAAAGAAAA |
| | | | | AACCGGGCTAGGATGGGGTCAGACGTTGAAGCGGA |
| | | | | TGCTCAGGATGTCGCCGTCCTTGACGCTG |
| MG178 | 7388 | MG178- | nucleo- | CAGCGAGCCGACCCCGTACAGGCTTAGAGCGCCGCC |
| recombinases | | 7199 AttL | tide | GGCATGGACCGGGCTGAAGCCGAGCTTCCGGGTCAG |
| attachment | | | | GTAGAGGGTCAGGAAAAAGATGACCATCGAGCCCG |
| sites | | | | AGGAATTGACCAGGTTGACCGTGAACAGGATCCAGG |
| | | | | CCTTGCGCGGCAGGCCGCTATTAGGGATTTCTTGGC |
| | | | | CGGCATTATTGCCACACCCGCCCATTTTTACTTGGAC |
| | | | | CGACTCAAAATTGTTTTGGAGGAATAAAATGAATGA |
| | | | | ATGTTTCGAATGCTGGCAAAACGCCAAAGAGGCCGG |
| | | | | CGAGATATCTGAATCGCAACATCAGC |
| MG178 | 7389 | MG178- | nucleo- | GTCGAGAAAGCCTATTCTCAGGCGATGCCTGAGCAC |
| recombinases | | 7199 AttP | tide | AGGGCCGAACTACTGCGCGTCCTCTTCGAGGACATT |
| attachment | | | | TCCATCAGCGGCGGAAAATTCGTCTTCACCCCACAG |
| sites | | | | GCCGTATTTGCCCCGCTCTTTGATTTAAGGCATGGCA |
| | | | | AACCACGGCAGGCCGCTATTAGGGATTTCTTGGCCG |
| | | | | GCATTATTGCCACACCCGCCCATTTTTACTTGGACCG |
| | | | | ACTCAAAATTGTTTTGGAGGAATAAAATGAATGAAT |
| | | | | GTTTCGAATGCTGGCAAAACGCCAAAGAGGCCGGCG |
| | | | | AGATATCTGAATCGCAACATCAGC |
| MG178 | 7390 | MG178- | nucleo- | GTCGAGAAAGCCTATTCTCAGGCGATGCCTGAGCAC |
| recombinases | | 7199 AttR | tide | AGGGCCGAACTACTGCGCGTCCTCTTCGAGGACATT |
| attachment | | | | TCCATCAGCGGCGGAAAATTCGTCTTCACCCCACAG |
| sites | | | | GCCGTATTTGCCCCGCTCTTTGATTTAAGGCATGGCA |
| | | | | AACCACGGCAGGCCGCTATACGCCTGGCGGTAGGTG |
| | | | | TCCCTGATCTTCCCGAGCATGGCCGTTAAAAGCGTA |
| | | | | TTACACCAGGACGGGTGGGAACGTCAAAGAAAAAA |
| | | | | CCGGGCTAGGATGGGGTCAGACGTTGAAGCGGATGC |
| | | | | TCAGGATGTCGCCGTCCTTGACGCTG |
| MG178 | 7391 | MG178- | nucleo- | CGGCAGGCCGCTAT |
| conserved | | 7199 Core | tide | |
| core | | | | |
| MG178 | 7392 | MG178- | nucleo- | CTCGTGGGCGGTCAACATCCGCAAGTACGTCGTGCG |
| recombinases | | 7200 AttB | tide | GGCGGACCACCTCGAGGACCTCGTCGCGCTCGGCTC |
| attachment | | | | GTTGCCGACCGATGCCGCGGCCTTCCTCTCGGCGGC |
| sites | | | | CGTGCGCGCCGGGTTGAACGTGCTCGTCTCCGGCGC |
| | | | | GACCCAAGGCGGGCAAGACGACGATGCTCAACGCG |
| | | | | CTCGCGGGCGCCGTGCCCGTGCGTGAGCGGGTCGTC |
| | | | | TCGTGCGAGGAGGTCTTCGAGCTGCGGCTCGCGGTC |
| | | | | CGGGACTGGGTCGCGATGCAGTGCCGTCAGCCCAAC |
| | | | | CTCGAGGGCACGGGGGAGA |
| MG178 | 7393 | MG178- | nucleo- | CTCGTGGGCGGTCAACATCCGCAAGTACGTCGTGCG |
| recombinases | | 7200 AttL | tide | GGCGGACCACCTCGAGGACCTCGTCGCGCTCGGCTC |
| attachment | | | | GTTGCCGACCGATGCCGCGGCCTTCCTCTCGGCGGC |
| sites | | | | CGTGCGCGCCGGGTTGAACGTGCTCGTCTCCGGCGC |
| | | | | GACCCAAGGCGGTAGTCTGCAAGCGCTTTACACAGT |
| | | | | ACGCTTGAGATCTCATGAACATGAGCGCCGGGCCCC |
| | | | | GAGCCGTCATCTACGTCCGCATCTCCGTTGCCCAGG |
| | | | | AGGCGTCGGTCTCCATCGAACGCCAGGTCGAGGCGG |
| | | | | CGGAACAGTACGCCGCTG |
| MG178 | 7394 | MG178- | nucleo- | GCGGGCTAGCTGATGAACAAGGAGACTAACAGCGG |
| recombinases | | 7200 AttP | tide | AGGTGAAAGCTTGTACAGAGATTAAGATCTAAATCC |
| attachment | | | | TGCAAGCTTCACCCGTTCGAGTGGTCGTCCCGAGTC |
| sites | | | | GATGAGAAGTGAGTAATACCTGGTTCACTACCGGCC |
| | | | | TACACGCAGGCGGTAGTCTGCAAGCGCTTTACACAG |
| | | | | TACGCTTGAGATCTCATGAACATGAGCGCCGGGCCC |
| | | | | CGAGCCGTCATCTACGTCCGCATCTCCGTTGCCCAG |
| | | | | GAGGCGTCGGTCTCCATCGAACGCCAGGTCGAGGCG |
| | | | | GCGGAACAGTACGCCGCTG |
| MG178 | 7395 | MG178- | nucleo- | GCGGGCTAGCTGATGAACAAGGAGACTAACAGCGG |
| recombinases | | 7200 AttR | tide | AGGTGAAAGCTTGTACAGAGATTAAGATCTAAATCC |
| attachment | | | | TGCAAGCTTCACCCGTTCGAGTGGTCGTCCCGAGTC |
| sites | | | | GATGAGAAGTGAGTAATACCTGGTTCACTACCGGCC |
| | | | | TACACGCAGGCGGGCAAGACGACGATGCTCAACGC |
| | | | | GCTCGCGGGCGCCGTGCCCGTGCGTGAGCGGGTCGT |
| | | | | CTCGTGCGAGGAGGTCTTCGAGCTGCGGCTCGCGGT |
| | | | | CCGGGACTGGGTCGCGATGCAGTGCCGTCAGCCCAA |
| | | | | CCTCGAGGGCACGGGGGAGA |
| MG178 | | MG178- | nucleo- | AGGCGG |
| conserved | | 7200 Core | tide | |
| core | | | | |
| MG178 | 7397 | MG178- | nucleo- | GCTTCAATAGCCTTAAAGTCATACTTGCTATTATGCT |
| recombinases | | 7203 AttB | tide | CCATAAACGTCCTCCTTCTGAATTATTATTGCCTATA |
| attachment | | | | AATAGCAAAAACTCTCATCCTGCTCAGGGACGAGAG |
| sites | | | | TATATTCCCGCGGTACCACCCTTATTGACAAATATAA |
| | | | | TAATGGTGGAGCTAGAGGGATTCGAACCCTCGACCT |
| | | | | CTTGAATGCCATTCAAGCGCGCTCCCAACTGCGCCA |
| | | | | TAGCCCCACATTTGTCCACTCTATTGATGATAACGGT |
| | | | | ATCTCCGTTACAGGCTTATCACCTGTAAAGCTTCCCG |
| | | | | GCGAGTTCGACATCTGCTGC |
| MG178 | 7398 | MG178- | nucleo- | GCTTCAATAGCCTTAAAGTCATACTTGCTATTATGCT |
| recombinases | | 7203 AttL | tide | CCATAAACGTCCTCCTTCTGAATTATTATTGCCTATA |
| attachment | | | | AATAGCAAAAACTCTCATCCTGCTCAGGGACGAGAG |
| sites | | | | TATATTCCCGCGGTACCACCCTTATTGACAAATATAA |
| | | | | TAATGGTGGAGCTAGACCGTCCCAGGAAGTATCCGC |
| | | | | ACAGTTACCATTCGCAGGCGCAGAGCAATTGATGGT |
| | | | | GGCACTCACCTTTGGCCGCCTCCAAGAGGCCAAGGC |
| | | | | GTTCGATATGCGTTAAACCCTCCGTCCATGCCACGCC |
| | | | | GAACGTGCCGTCCCTGTACTT |
| MG178 | 7399 | MG178- | nucleo- | TGAACCATGTTAGCGAGGCGCTTAAAGAAGCCAAAA |
| recombinases | | 7203 AttP | tide | GCCCGGCTGAGTACCGGGCCGCGATCCACCGCTTCA |
| attachment | | | | TCGACCGGATAGTGGTCGGCGAAAAAATGATCCAAA |
| sites | | | | TCCACTTCCTGGCCGACTTCGGCGGCGGTGTGTGGA |
| | | | | TAAAGTTGGTGGAGCTAGACCGTCCCAGGAAGTATC |
| | | | | CGCACAGTTACCATTCGCAGGCGCAGAGCAATTGAT |
| | | | | GGTGGCACTCACCTTTGGCCGCCTCCAAGAGGCCAA |
| | | | | GGCGTTCGATATGCGTTAAACCCTCCGTCCATGCCA |
| | | | | CGCCGAACGTGCCGTCCCTGTACTT |
| MG178 | 7400 | MG178- | nucleo- | TGAACCATGTTAGCGAGGCGCTTAAAGAAGCCAAAA |
| recombinases | | 7203 AttR | tide | GCCCGGCTGAGTACCGGGCCGCGATCCACCGCTTCA |
| attachment | | | | TCGACCGGATAGTGGTCGGCGAAAAAATGATCCAAA |
| sites | | | | TCCACTTCCTGGCCGACTTCGGCGGCGGTGTGTGGA |
| | | | | TAAAGTTGGTGGAGCTAGAGGGATTCGAACCCTCGA |
| | | | | CCTCTTGAATGCCATTCAAGCGCGCTCCCAACTGCG |
| | | | | CCATAGCCCCACATTTGTCCACTCTATTGATGATAAC |
| | | | | GGTATCTCCGTTACAGGCTTATCACCTGTAAAGCTTC |
| | | | | CCGGCGAGTTCGACATCTGCTGC |
| MG178 | 7401 | MG178- | nucleo- | TGGTGGAGCTAGA |
| conserved | | 7203 Core | tide | |
| core | | | | |
| MG178 | 7402 | MG178- | nucleo- | GCTGGCCAAACGCCTGCCCACCATATTACCACCACT |
| recombinases | | 7204 AttB | tide | TACTTTGCATGAAGCGCTTGAAACCACAAAGATTCA |
| attachment | | | | CAGTGTAGCAGGAAAACTACCCGAAAATGCCACATT |
| sites | | | | GATTTCAAAAAGACCTTTTCGCAGCCCGCACCATAC |
| | | | | CGTTTCGGATGCGGCTTTGGTTGGTGGCGGCAGCAC |
| | | | | CCCGCAACCGGGGGAAATTTCACTGGCACATAATGG |
| | | | | CGTATTATTTTTAGACGAATTGCCTGAATTCAAAAG |
| | | | | AACCGCGCTGGAAGTGATGCGCCAGCCCATGGAAG |
| | | | | AGAGAAAAGTAACTATCAG |
| MG178 | 7403 | MG178- | nucleo- | GCTGGCCAAACGCCTGCCCACCATATTACCACCACT |
| recombinases | | 7204 AttL | tide | TACTTTGCATGAAGCGCTTGAAACCACAAAGATTCA |
| attachment | | | | CAGTGTAGCAGGAAAACTACCCGAAAATGCCACATT |
| sites | | | | GATTTCAAAAAGACCTTTTCGCAGCCCGCACCATAC |
| | | | | CGTTTCGGATGCTATGATATTACAGTAGGCGGTCGT |
| | | | | ATATTGTGAAAAATAAGACTATTTTTCAATGAAACG |
| | | | | TATAGCCATCTACAGCCGCGTATCAACAGCAGATAA |
| | | | | ACAGGATTACACAAGGCAGGTTAACGAACTTAAGA |
| | | | | AGATTGGTTACGATAACGG |
| MG178 | 7404 | MG178- | nucleo- | CAGGCGTAAGAGATTTTACCATATTTCAAGGATTTT |
| recombinases | | 7204 AttP | tide | AGGTTTATGAACACCTAAATTTAAGAAAAACAGCCA |
| attachment | | | | AAATATTTTATTAGCTAAACCCTTATCTGTAGCGGGT |
| sites | | | | TTCAACGCCAGTTTCACCACCCCCCACTAAAATGCC |
| | | | | GCTGGGGATGCTATGATATTACAGTAGGCGGTCGTA |
| | | | | TATTGTGAAAAATAAGACTATTTTTCAATGAAACGT |
| | | | | ATAGCCATCTACAGCCGCGTATCAACAGCAGATAAA |
| | | | | CAGGATTACACAAGGCAGGTTAACGAACTTAAGAA |
| | | | | GATTGGTTACGATAACGG |
| MG178 | 7405 | MG178- | nucleo- | CAGGCGTAAGAGATTTTACCATATTTCAAGGATTTT |
| recombinases | | 7204 AttR | tide | AGGTTTATGAACACCTAAATTTAAGAAAAACAGCCA |
| attachment | | | | AAATATTTTATTAGCTAAACCCTTATCTGTAGCGGGT |
| sites | | | | TTCAACGCCAGTTTCACCACCCCCCACTAAAATGCC |
| | | | | GCTGGGGATGCGGCTTTGGTTGGTGGCGGCAGCACC |
| | | | | CCGCAACCGGGGGAAATTTCACTGGCACATAATGGC |
| | | | | GTATTATTTTTAGACGAATTGCCTGAATTCAAAAGA |
| | | | | ACCGCGCTGGAAGTGATGCGCCAGCCCATGGAAGA |
| | | | | GAGAAAAGTAACTATCAG |
| MG178 | | MG178- | nucleo- | GGATGC |
| conserved | | 7204 Core | tide | |
| core | | | | |
| Primer | 7407 | IVT | nucleo- | CCCTTCACCTTCTATCTCGAAC |
| | | Junction rev | tide | |
| Primer | 7408 | IVT 47 | nucleo- | ACTCGGCCTTGGCACT |
| | | junction fwd | tide | |
| Primer | 7409 | IVT 37 | nucleo- | GCCGAGAACCTTGTCTTCC |
| | | junction fwd | tide | |
| Primer | 7410 | IVT 36 | nucleo- | ACAGCCGTTTTGACTGGA |
| | | junction fwd | tide | |
| Primer | 7411 | IVT 20 | nucleo- | AGGTACACCTCCTGCAGC |
| | | junction fwd | tide | |
| MG178 | 7412 | Sumo Tag | protein | SDSEVNQEAKPEVKPEVKPETHINLKVSDGSSEIFFKIK |
| recombinases | | | | KTTPLRRLMEAFAKRQGKEMDSLRFLYDGIRIQADQTP |
| protein tag | | | | EDLDMEDNDIIEAHREQIGG |
| MG178 | 7413 | Hexahistadine | protein | HHHHHH |
| recombinases | | Tag | | |
| protein tag | | | | |
| MG178 | 7414 | SV40 NLS | protein | KKKRKV |
| recombinases | | | | |
| protein tag | | | | |
| MG178 | 7415 | HA | protein | YPYDVPDYA |
| recombinases | | | | |
| protein tag | | | | |
| Primer | 7416 | mcherry rev | nucleo- | AACTCCTTGATGATGGCC |
| | | | tide | |
| Primer | 7417 | pCMV | nucleo- | CGATGGATAGCGATTTTATTATC |
| | | mNeon fwd | tide | |
| MG178 | 7418 | PS protease | protein | LEVLFQGP |
| recombinases | | cleavage site | | |
| protein tag | | | | |

1. A gene editing system comprising:
a) a serine recombinase comprising at least about 80% sequence identity to any one of SEQ ID NOs: 21-7060, 7105-7142, and 7211-7214 or a nucleic acid encoding the serine recombinase; and
b) a nucleic acid comprising a donor polynucleotide and a first attachment site sequence.

2. The gene editing system of claim 1, wherein the first attachment site sequence is 5′ of the donor polynucleotide.

3. The gene editing system of any one of claims 1-2, wherein the nucleic acid encoding the serine recombinase further comprises a second attachment site sequence.

4. The gene editing system of claim 3, wherein the second attachment site sequence is 5′ of the serine recombinase.

5. The gene editing system of any one of claims 3-4, wherein the first attachment site sequence and the second attachment site sequence are capable of recombination.

6. The gene editing system of any one of claims 1-5, wherein the first attachment site sequence is a bacterial genomic recombination sequence (attB).

7. The gene editing system of any one of claims 1-5, wherein the first attachment site sequence is a phage genomic recombination sequence (attP).

8. The gene editing system of any one of claims 3-7, wherein the second attachment site sequence is a bacterial genomic recombination sequence (attB).

9. The gene editing system of any one of claims 3-7, wherein the second attachment site sequence is a phage genomic recombination sequence (attP).

10. The gene editing system of any one of claims 6-9, wherein the attB sequence comprises about 20 to about 500 nucleotides.

11. The gene editing system of any one of claims 7-10, wherein the attP sequence comprises about 20 to about 500 nucleotides.

12. The gene editing system of any one of claims 6-11, wherein the attB sequence comprises at least about 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402.

13. The gene editing system of any one of claims 6-12, wherein the attB sequence comprises at least about 80% sequence identity to any one of SEQ ID NOs: 1, 5, 9, and 13.

14. The gene editing system of any one of claims 7-13, wherein the attP sequence comprises at least about 80% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404.

15. The gene editing system of any one of claims 7-14, wherein the attP sequence comprises at least about 80% sequence identity to any one of SEQ ID NOs: 2, 6, 10, and 14.

16. The gene editing system of any one of claims 1-15, wherein the nucleic acid comprising the donor polynucleotide and the first attachment sequence is delivered using plasmid, a nanoplasmid, a phagemid, a phage derivative, a virus, a bacmid, a bacterial artificial chromosome (BAC), a minicircle, a doggybone, a yeast artificial chromosome (YAC), or a cosmid.

17. The gene editing system of any one of claims 1-16, wherein the nucleic acid encoding the serine recombinase is delivered using a plasmid, a nanoplasmid, a phagemid, a phage derivative, a virus, a bacmid, a bacterial artificial chromosome (BAC), a minicircle, a doggybone, a yeast artificial chromosome (YAC), or a cosmid.

18. The gene editing system of any one of claims 16-17, wherein the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus.

19. The gene editing system of claim 18, wherein the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof.

20. The gene editing system of claim 18, wherein the herpesvirus is HSV-1, HSV-2, VZV, EBV, CMV, HHV-6, HHV-7, or HHV-8.

21. The gene editing system of any one of claims 1-20, wherein the donor polynucleotide comprises a size of at least about 1 kilobase (kb), 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 110 kb, 120 kb, or more than 120 kb.

22. The gene editing system of any one of claims 1-21, wherein the donor polynucleotide encodes a therapeutic, a reporter, or a marker.

23. The gene editing system of claim 22, wherein the reporter comprises a fluorescent protein.

24. The gene editing system of claim 23, wherein the fluorescent protein is GFP, EBFP, EBFP2, Azurite, mKalamal, ECFP, Cerulean, CyPet, YFP, Citrine, Venus, YPet, RFP, CFP, or a derivative thereof.

25. The gene editing system of claim 22, wherein the reporter is acetohydroxyacid synthase (AHAS), alkaline phosphatase (AP), beta galactosidase (LacZ), beta glucuronidase (GUS), chloramphenicol acetyltransferase (CAT), horseradish peroxidase (HRP), luciferase (Luc), nopaline synthase (NOS), octopine synthase (OCS), luciferase, or a derivative thereof.

26. The gene editing system of any one of claims 22-25, wherein the marker is an antibiotic resistance marker.

27. The gene editing system of claim 26, wherein the antibiotic resistance marker is kanamycin, spectinomycin, streptomycin, ampicillin, carbenicillin, bleomycin, erythromycin, polymyxin B, tetracycline, chloramphenicol, neomycin, zeocin, or a derivative thereof.

28. The gene editing system of any one of claims 22-27, wherein the marker is a cell surface marker.

29. A eukaryotic genome comprising a donor polynucleotide sequence; and an attL sequence 5′ to the donor polynucleotide sequence, wherein the attL sequence comprises a sequence selected from the group consisting of: SEQ ID NOs: 3, 4, 7, 8, 11, 12, 15, 16, 7153, 7157, 7161, 7165, 7169, 7173, 7177, 7181, 7216, 7221, 7227, 7234, 7239, 7244, 7249, 7254, 7259, 7265, 7272, 7278, 7283, 7288, 7293, 7298, 7303, 7308, 7313, 7318, 7323, 7328, 7333, 7338, 7343, 7348, 7353, 7358, 7363, 7368, 7373, 7378, 7383, 7388, 7393, 7398, and 7403.

30. The eukaryotic genome of claim 29, further comprising an attR sequence 3′ to the donor polynucleotide sequence.

31. A eukaryotic genome comprising a donor polynucleotide sequence; and an attL sequence 3′ to the donor polynucleotide sequence, wherein the attL sequence comprises a sequence selected from the group consisting of: SEQ ID NOs: 3, 4, 7, 8, 11, 12, 15, 16, 7153, 7157, 7161, 7165, 7169, 7173, 7177, 7181, 7216, 7221, 7227, 7234, 7239, 7244, 7249, 7254, 7259, 7265, 7272, 7278, 7283, 7288, 7293, 7298, 7303, 7308, 7313, 7318, 7323, 7328, 7333, 7338, 7343, 7348, 7353, 7358, 7363, 7368, 7373, 7378, 7383, 7388, 7393, 7398, and 7403.

32. The eukaryotic genome of claim 31, further comprising an attR sequence 3′ to the donor polynucleotide sequence.

33. A eukaryotic genome comprising:
a donor polynucleotide sequence;
an attL sequence 5′ or 3′ to the donor polynucleotide sequence, wherein the attL sequence comprises a sequence selected from the group consisting of: SEQ ID NOs: 3, 4, 7, 8, 11, 12, 15, 16, 7153, 7157, 7161, 7165, 7169, 7173, 7177, 7181, 7216, 7221, 7227, 7234, 7239, 7244, 7249, 7254, 7259, 7265, 7272, 7278, 7283, 7288, 7293, 7298, 7303, 7308, 7313, 7318, 7323, 7328, 7333, 7338, 7343, 7348, 7353, 7358, 7363, 7368, 7373, 7378, 7383, 7388, 7393, 7398, and 7403; and
an attR sequence 5′ or 3′ to the donor polynucleotide sequence, wherein the attR sequence comprises a sequence selected from the group consisting of: SEQ ID NOs: 3, 4, 7, 8, 11, 12, 15, 16, 7154, 7158, 7162, 7166, 7170, 7174, 7178, 7182, 7218, 7223, 7230, 7236, 7241, 7246, 7251, 7256, 7261, 7268, 7274, 7280, 7285, 7290, 7295, 7300, 7305, 7310, 7315, 7320, 7325, 7330, 7335, 7340, 7345, 7350, 7355, 7360, 7365, 7370, 7375, 7380, 7385, 7390, 7395, 7400, and 7405.

34. The eukaryotic genome of any one of claim 30, 32, or 33, wherein the attL sequence and the attR sequence are the same.

35. The eukaryotic genome of any one of claims 29-34, wherein the attL sequence is a recombined sequence of a first attachment site sequence and a second attachment site sequence.

36. The eukaryotic genome of any one of claim 30 or 32-35, wherein the attR sequence is a recombined sequence of a first attachment site sequence and a second attachment site sequence.

37. The eukaryotic genome of any one of claims 35-36, wherein the first attachment site sequence is a bacterial genomic recombination sequence (attB).

38. The eukaryotic genome of any one of claims 35-36, wherein the first attachment site sequence is a phage genomic recombination sequence (attP).

39. The eukaryotic genome of any one of claims 35-38, wherein the second attachment site sequence is a bacterial genomic recombination sequence (attB).

40. The eukaryotic genome of any one of claims 35-38, wherein the second attachment site sequence is a phage genomic recombination sequence (attP).

41. The eukaryotic genome of any one of claims 37-40, wherein the attB sequence comprises about 20 to about 500 nucleotides.

42. The eukaryotic genome of any one of claims 38-41, wherein the attP sequence comprises about 20 to about 500 nucleotides.

43. The eukaryotic genome of any one of claims 37-42, wherein the attB sequence comprises at least about 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402.

44. The eukaryotic genome of any one of claims 37-43, wherein the attB sequence comprises at least about 80% sequence identity to any one of SEQ ID NOs: 1, 5, 9, and 13.

45. The eukaryotic genome of any one of claims 38-44, wherein the attP sequence comprises at least about 80% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404.

46. The eukaryotic genome of any one of claims 38-45, wherein the attP sequence comprises at least about 80% sequence identity to any one of SEQ ID NOs: 2, 6, 10, and 14.

47. The eukaryotic genome of any one of claims 29-46, wherein the attL sequence comprises at least about 80% sequence identity to any one of SEQ ID NOs: 3, 4, 7, 8, 11, 12, 15, 16, 7153, 7157, 7161, 7165, 7169, 7173, 7177, 7181, 7216, 7221, 7227, 7234, 7239, 7244, 7249, 7254, 7259, 7265, 7272, 7278, 7283, 7288, 7293, 7298, 7303, 7308, 7313, 7318, 7323, 7328, 7333, 7338, 7343, 7348, 7353, 7358, 7363, 7368, 7373, 7378, 7383, 7388, 7393, 7398, and 7403.

48. The eukaryotic genome of any one of claims 29-47, wherein the attR sequence comprises at least about 80% sequence identity to any one of SEQ ID NOs: 3, 4, 7, 8, 11, 12, 15, 16, 7154, 7158, 7162, 7166, 7170, 7174, 7178, 7182, 7218, 7223, 7230, 7236, 7241, 7246, 7251, 7256, 7261, 7268, 7274, 7280, 7285, 7290, 7295, 7300, 7305, 7310, 7315, 7320, 7325, 7330, 7335, 7340, 7345, 7350, 7355, 7360, 7365, 7370, 7375, 7380, 7385, 7390, 7395, 7400, and 7405.

49. A mammalian cell comprising the eukaryotic genome of any one of claims 29-48.

50. The mammalian cell of claim 49, wherein the mammalian cell is a human cell.

51. The mammalian cell of any one of claims 49-50, further comprising a serine recombinase.

52. The mammalian cell of claim 51, wherein the serine recombinase comprises at least about 80% sequence identity to any one of SEQ ID NOs: 21-7060, 7105-7142, and 7211-7214.

53. The mammalian cell of claim 51, wherein the serine recombinase comprises at least about 80% sequence identity to SEQ ID NO: 21.

54. The mammalian cell of claim 51, wherein the serine recombinase comprises at least about 80% sequence identity to SEQ ID NO: 22.

55. The mammalian cell of claim 51, wherein the serine recombinase comprises at least about 80% sequence identity to SEQ ID NO: 23.

56. The mammalian cell of claim 51, wherein the serine recombinase comprises at least about 80% sequence identity to SEQ ID NO: 24.

57. The mammalian cell of claim 51, wherein the serine recombinase comprises an integration efficiency of at least about 5%.

58. The mammalian cell of claim 51, wherein the serine recombinase comprises an integration efficiency of at least about 25%.

59. The mammalian cell of claim 51, wherein the serine recombinase comprises an integration efficiency of at least about 50%.

60. The mammalian cell of claim 51, wherein the serine recombinase is capable of targeting genes comprising a catalase domain or synthase domain.

61. The mammalian cell of claim 60, wherein the catalase is manganese catalase.

62. The mammalian cell of any one of claims 60-61, wherein the synthase is Queuosine synthase.

63. The mammalian cell of any one of claims 60-62, wherein the serine recombinase is capable of targeting genes comprising a DUF4244 Pfam domain.

64. A eukaryotic cell comprising a serine recombinase comprising at least about 80% sequence identity to any one of SEQ ID NOs: 21-7060, 7105-7142, and 7211-7214.

65. A eukaryotic cell comprising a serine recombinase comprising at least about 80% sequence identity to SEQ ID NO: 21.

66. A eukaryotic cell comprising a serine recombinase comprising at least about 80% sequence identity to SEQ ID NO: 22.

67. A eukaryotic cell comprising a serine recombinase comprising at least about 80% sequence identity to SEQ ID NO: 23.

68. A eukaryotic cell comprising a serine recombinase comprising at least about 80% sequence identity to SEQ ID NO: 24.

69. A eukaryotic cell comprising a serine recombinase comprising at least about 80% sequence identity to SEQ ID NO: 1848.

70. A eukaryotic cell comprising a serine recombinase comprising at least about 80% sequence identity to SEQ ID NO: 7111.

71. A eukaryotic cell comprising a serine recombinase comprising at least about 80% sequence identity to SEQ ID NO: 7115.

72. A eukaryotic cell comprising a serine recombinase comprising at least about 80% sequence identity to SEQ ID NO: 7131.

73. A eukaryotic cell comprising a serine recombinase comprising at least about 80% sequence identity to SEQ ID NO: 7136.

74. A eukaryotic cell comprising a serine recombinase comprising at least about 80% sequence identity to SEQ ID NO: 7139.

75. A eukaryotic cell comprising a serine recombinase comprising at least about 80% sequence identity to SEQ ID NO: 7140.

76. The eukaryotic cell of any one of claims 64-75, wherein the eukaryotic cell is a mammalian cell.

77. The eukaryotic cell of any one of claims 64-75, wherein the eukaryotic cell is a human cell.

78. A vector comprising:
a) a nucleic acid encoding serine recombinase comprising at least about 80% sequence identity to any one of SEQ ID NOs: 21-7060, 7105-7142, and 7211-7214; and
b) one or more regulatory elements.

79. The vector of claim 78, wherein the one or more regulatory elements comprises a promoter, an enhancer, an intron, a microRNA, a linker, a splicing element, or a polyA signal.

80. The vector of claim 79, wherein the promoter is selected from a constitutive promoter, an inducible promoter, a mini promoter, or a derivative thereof.

81. The vector of claim 79, wherein the promoter is selected from the group consisting of:
CMV, CBA, EF1a, CAG, PGK, TRE, U6, UAS, T7, Sp6, lac, araBad, trp, Ptac, p5, p19, p40, Synapsin, CaMKII, GRK1, polH, EM7, OpIE1, and a derivative thereof.

82. A vector comprising a nucleic acid encoding a serine recombinase comprising at least about 80% sequence identity to any one of SEQ ID NOs: 21-7060, 7105-7142, and 7211-7214, wherein the vector is selected from the group consisting of: a plasmid, a nanoplasmid, a phagemid, a phage derivative, a bacmid, a bacterial artificial chromosome (BAC), a minicircle, a doggybone, a yeast artificial chromosome (YAC), and a cosmid.

83. A method for gene editing, comprising:
a) providing or identifying a first attachment site sequence in a host genome;
b) providing a nucleic acid comprising a donor polynucleotide and a second attachment site sequence to a host cell; and
c) contacting the host cell with a serine recombinase comprising at least about 80% sequence identity to any one of SEQ ID NOs: 21-7060, 7105-7142, and 7211-7214 or a nucleic acid encoding the serine recombinase,
wherein the first attachment site sequence and the second attachment site sequence are capable of recombination.

84. The method of claim 83, wherein the first attachment site sequence is endogenous in the host genome.

85. The method of claim 83, wherein the first attachment site sequence is provided using viral delivery.

86. The method of claim 83, wherein the first attachment site sequence is provided using a transposase.

87. The method of claim 83, wherein the first attachment site sequence is provided using a nuclease.

88. The method of claim 87, wherein the nuclease is a double-strand nuclease.

89. The method of claim 87, wherein the nuclease is a Type II CRISPR endonuclease.

90. The method of claim 87, wherein the nuclease is a Type V CRISPR endonuclease.

91. The method of claim 87, wherein the nuclease is Cas9.

92. The method of claim 76, wherein the first attachment site sequence is provided using a reverse transcriptase.

93. The method of any one of claims 83-92, wherein the second attachment site sequence is 5′ of the donor polynucleotide.

94. The method of any one of claims 83-93, wherein the first attachment site sequence is a bacterial genomic recombination sequence (attB).

95. The method of any one of claims 83-94, wherein the first attachment site sequence is a phage genomic recombination sequence (attP).

96. The method of any one of claims 83-95, wherein the second attachment site sequence is a bacterial genomic recombination sequence (attB).

97. The method of any one of claims 83-96, wherein the second attachment site sequence is a phage genomic recombination sequence (attP).

98. The method of any one of claims 94-97, wherein the attB sequence comprises about 20 to about 500 nucleotides.

99. The method of any one of claims 95-98, wherein the attP sequence comprises about 20 to about 500 nucleotides.

100. The method of any one of claims 94-99, wherein the attB sequence comprises at least about 80% sequence identity to any one of SEQ ID NOs: 1, 2, 5, 6, 9, 10, 13, 14, 7151, 7155, 7159, 7163, 7167, 7171, 7175, 7179, 7188-7200, 7206-7210, 7215, 7220, 7225, 7226, 7233, 7238, 7243, 7248, 7253, 7258, 7263, 7264, 7271, 7277, 7282, 7287, 7292, 7297, 7302, 7307, 7312, 7317, 7322, 7327, 7332, 7337, 7342, 7347, 7352, 7357, 7362, 7367, 7372, 7377, 7382, 7387, 7392, 7397, and 7402.

101. The method of any one of claims 94-100, wherein the attB sequence comprises at least about 80% sequence identity to any one of SEQ ID NOs: 1, 5, 9, and 13.

102. The method of any one of claims 95-101, wherein the attP sequence comprises at least about 80% sequence identity to any one of SEQ ID NOs: 1, 2, 5, 6, 9, 10, 13, 14, 7152, 7156, 7160, 7164, 7168, 7172, 7176, 7180, 7183-7187, 7201-7205, 7217, 7222, 7228, 7229, 7235, 7240, 7245, 7250, 7255, 7260, 7266, 7267, 7273, 7279, 7284, 7289, 7294, 7299, 7304, 7309, 7314, 7319, 7324, 7329, 7334, 7339, 7344, 7349, 7354, 7359, 7364, 7369, 7374, 7379, 7384, 7389, 7394, 7399, and 7404.

103. The method of any one of claims 95-102, wherein the attP sequence comprises at least about 80% sequence identity to any one of SEQ ID NOs: 2, 6, 10, and 14.

104. The method of any one of claims 83-103, wherein the nucleic acid comprising the donor polynucleotide and the second attachment site sequence is delivered by a plasmid, a nanoplasmid, a phagemid, a phage derivative, a virus, a bacmid, a bacterial artificial chromosome (BAC), a minicircle, a doggybone, a yeast artificial chromosome (YAC), or a cosmid.

105. The method of any one of claims 83-106, wherein the nucleic acid encoding the serine recombinase is delivered by a plasmid, a nanoplasmid, a phagemid, a phage derivative, a virus, a bacmid, a bacterial artificial chromosome (BAC), a minicircle, a doggybone, a yeast artificial chromosome (YAC), or a cosmid.

106. The method of any one of claims 104-105, wherein the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus.

107. The method of claim 106, wherein the AAV is AAV1, AAV2, AAV3, AAV4, AAV5, AAV6, AAV7, AAV8, AAV9, AAV10, AAV11, AAV12, AAV13, AAV14, AAV15, AAV16, AAV-rh8, AAV-rh10, AAV-rh20, AAV-rh39, AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14, AAV-HSC15, AAV-TT, AAV-DJ/8, AAV-Myo, AAV-NP40, AAV-NP59, AAV-NP22, AAV-NP66, or AAV-HSC16, or a derivative thereof.

108. The method of claim 106, wherein the herpesvirus is HSV-1, HSV-2, VZV, EBV, CMV, HHV-6, HHV-7, or HHV-8.

109. The method of any one of claims 83-108, wherein the donor polynucleotide comprises a size of at least about 1 kilobase (kb), 2 kb, 3 kb, 4 kb, 5 kb, 6 kb, 7 kb, 8 kb, 9 kb, 10 kb, 20 kb, 30 kb, 40 kb, 50 kb, 60 kb, 70 kb, 80 kb, 90 kb, 100 kb, 110 kb, 120 kb, or more than 120 kb.

110. The method of any one of claims 83-109, wherein the donor polynucleotide encodes a therapeutic, a reporter, or a marker.

111. The method of claim 110, wherein the reporter comprises a fluorescent protein.

112. The method of claim 111, wherein the fluorescent protein is GFP, EBFP, EBFP2, Azurite, mKalamal, ECFP, Cerulean, CyPet, YFP, Citrine, Venus, YPet, RFP, CFP, or a derivative thereof.

113. The method of claim 110, wherein the reporter is acetohydroxyacid synthase (AHAS), alkaline phosphatase (AP), beta galactosidase (LacZ), beta glucuronidase (GUS), chloramphenicol acetyltransferase (CAT), horseradish peroxidase (HRP), luciferase (Luc), nopaline synthase (NOS), octopine synthase (OCS), luciferase, or a derivative thereof.

114. The method of any one of claims 110-113, wherein the marker is an antibiotic resistance marker.

115. The method of claim 114, wherein the antibiotic resistance marker is kanamycin, spectinomycin, streptomycin, ampicillin, carbenicillin, bleomycin, erythromycin, polymyxin B, tetracycline, chloramphenicol, neomycin, zeocin, or a derivative thereof.

116. The method of any one of claims 110-113, wherein the marker is a cell surface marker.
